SEQUENCE LISTING (67 sequences)

SEQ ID NO: 1 - DNA, 41 bp, Artificial Sequence
  /note=“Description of Artificial Sequence Synthetic primer”
aatggactatcatatgcttaccgtaacttgaaagtatttcg

SEQ ID NO: 2 - DNA, 44 bp, Artificial Sequence
  /note=“Description of Artificial Sequence Synthetic primer”
ctttagtttgtatgtctgttgctattatgtctactattctttcc

SEQ ID NO: 3 - DNA, 89 bp, Artificial Sequence
  /note=“Description of Artificial Sequence Synthetic primer”
caagcagaagacggcatacgagatcctctctggtgactggagttcagacgtgtgctcttccgatcttctactattctttcccctgcact

SEQ ID NO: 4 - DNA, 89 bp, Artificial Sequence
  /note=“Description of Artificial Sequence Synthetic primer”
caagcagaagacggcatacgagatctagtacggtgactggagttcagacgtgtgctcttccgatcttctactattctttcccctgcact

SEQ ID NO: 5 - DNA, 89 bp, Artificial Sequence
  /note=“Description of Artificial Sequence Synthetic primer”
caagcagaagacggcatacgagatttctgcctgtgactggagttcagacgtgtgctcttccgatcttctactattctttcccctgcact

SEQ ID NO: 6 - DNA, 89 bp, Artificial Sequence
  /note=“Description of Artificial Sequence Synthetic primer”
caagcagaagacggcatacgagatgctcaggagtgactggagttcagacgtgtgctcttccgatcttctactattctttcccctgcact

SEQ ID NO: 7 - DNA, 89 bp, Artificial Sequence
  /note=“Description of Artificial Sequence Synthetic primer”
caagcagaagacggcatacgagataggagtccgtgactggagttcagacgtgtgctcttccgatcttctactattctttcccctgcact

SEQ ID NO: 8 - DNA, 89 bp, Artificial Sequence
  /note=“Description of Artificial Sequence Synthetic primer”
caagcagaagacggcatacgagatcatgcctagtgactggagttcagacgtgtgctcttccgatcttctactattctttcccctgcact

SEQ ID NO: 9 - DNA, 89 bp, Artificial Sequence
  /note=“Description of Artificial Sequence Synthetic primer”
caagcagaagacggcatacgagatgtagagaggtgactggagttcagacgtgtgctcttccgatcttctactattctttcccctgcact

SEQ ID NO: 10 - DNA, 89 bp, Artificial Sequence
  /note=“Description of Artificial Sequence Synthetic primer”
caagcagaagacggcatacgagataacaatgggtgactggagttcagacgtgtgctcttccgatcttctactattctttcccctgcact

SEQ ID NO: 11 - DNA, 89 bp, Artificial Sequence
  /note=“Description of Artificial Sequence Synthetic primer”
caagcagaagacggcatacgagatagcgtagcgtgactggagttcagacgtgtgctcttccgatcttctactattctttcccctgcact

SEQ ID NO: 12 - DNA, 89 bp, Artificial Sequence
  /note=“Description of Artificial Sequence Synthetic primer”
caagcagaagacggcatacgagatcagcctcggtgactggagttcagacgtgtgctcttccgatcttctactattctttcccctgcact

SEQ ID NO: 13 - DNA, 89 bp, Artificial Sequence
  /note=“Description of Artificial Sequence Synthetic primer”
caagcagaagacggcatacgagatagtagcgtgtgactggagttcagacgtgtgctcttccgatcttctactattctttcccctgcact

SEQ ID NO: 14 - DNA, 89 bp, Artificial Sequence
  /note=“Description of Artificial Sequence Synthetic primer”
caagcagaagacggcatacgagatcagtgagtgtgactggagttcagacgtgtgctcttccgatcttctactattctttcccctgcact

SEQ ID NO: 15 - DNA, 89 bp, Artificial Sequence
  /note=“Description of Artificial Sequence Synthetic primer”
caagcagaagacggcatacgagatcgtactcagtgactggagttcagacgtgtgctcttccgatcttctactattctttcccctgcact

SEQ ID NO: 16 - DNA, 89 bp, Artificial Sequence
  /note=“Description of Artificial Sequence Synthetic primer”
caagcagaagacggcatacgagatctacgcaggtgactggagttcagacgtgtgctcttccgatcttctactattctttcccctgcact

SEQ ID NO: 17 - DNA, 89 bp, Artificial Sequence
  /note=“Description of Artificial Sequence Synthetic primer”
caagcagaagacggcatacgagatggagactagtgactggagttcagacgtgtgctcttccgatcttctactattctttcccctgcact

SEQ ID NO: 18 - DNA, 89 bp, Artificial Sequence
  /note=“Description of Artificial Sequence Synthetic primer”
caagcagaagacggcatacgagataggtaagggtgactggagttcagacgtgtgctcttccgatcttctactattctttcccctgcact

SEQ ID NO: 19 - DNA, 89 bp, Artificial Sequence
  /note=“Description of Artificial Sequence Synthetic primer”
caagcagaagacggcatacgagataacgcattgtgactggagttcagacgtgtgctcttccgatcttctactattctttcccctgcact

SEQ ID NO: 20 - DNA, 60 bp, Artificial Sequence
  /note=“Description of Artificial Sequence Synthetic primer”
caagcagaagacggcatacgagatacaggtatgtgactggagttcagacgtgtgctcttc

SEQ ID NO: 21 - DNA, 82 bp, Artificial Sequence
  /note=“Description of Artificial Sequence Synthetic primer”
aatgatacggcgaccaccgagatctacactctttccctacacgacgctcttccgatcttcttgtggaaaggacgaaacaccg

SEQ ID NO: 22 - DNA, 83 bp, Artificial Sequence
  /note=“Description of Artificial Sequence Synthetic primer”
aatgatacggcgaccaccgagatctacactctttccctacacgacgctcttccgatctatcttgtggaaaggacgaaacaccg

SEQ ID NO: 23 - DNA, 84 bp, Artificial Sequence
  /note=“Description of Artificial Sequence Synthetic primer”
aatgatacggcgaccaccgagatctacactctttccctacacgacgctcttccgatcttatcttgtggaaaggacgaaacaccg

SEQ ID NO: 24 - DNA, 85 bp, Artificial Sequence
  /note=“Description of Artificial Sequence Synthetic primer”
aatgatacggcgaccaccgagatctacactctttccctacacgacgctcttccgatctgtatcttgtggaaaggacgaaacaccg

SEQ ID NO: 25 - DNA, 86 bp, Artificial Sequence
  /note=“Description of Artificial Sequence Synthetic primer”
aatgatacggcgaccaccgagatctacactctttccctacacgacgctcttccgatctcgtatcttgtggaaaggacgaaacaccg

SEQ ID NO: 26 - DNA, 87 bp, Artificial Sequence
  /note=“Description of Artificial Sequence Synthetic primer”
aatgatacggcgaccaccgagatctacactctttccctacacgacgctcttccgatctacgtatcttgtggaaaggacgaaacaccg

SEQ ID NO: 27 - DNA, 88 bp, Artificial Sequence
  /note=“Description of Artificial Sequence Synthetic primer”
aatgatacggcgaccaccgagatctacactctttccctacacgacgctcttccgatctgacgtatcttgtggaaaggacgaaacaccg

SEQ ID NO: 28 - DNA, 89 bp, Artificial Sequence
  /note=“Description of Artificial Sequence Synthetic primer”
aatgatacggcgaccaccgagatctacactctttccctacacgacgctcttccgatctagacgtatcttgtggaaaggacgaaacaccg

SEQ ID NO: 29 - DNA, 90 bp, Artificial Sequence
  /note=“Description of Artificial Sequence Synthetic primer”
aatgatacggcgaccaccgagatctacactctttccctacacgacgctcttccgatctcagacgtatcttgtggaaaggacgaaacaccg

SEQ ID NO: 30 - DNA, 91 bp, Artificial Sequence
  /note=“Description of Artificial Sequence Synthetic primer”
aatgatacggcgaccaccgagatctacactctttccctacacgacgctcttccgatcttcagacgtatcttgtggaaaggacgaaacaccg

SEQ ID NO: 31 - DNA, 92 bp, Artificial Sequence
  /note=“Description of Artificial Sequence Synthetic primer”
aatgatacggcgaccaccgagatctacactctttccctacacgacgctcttccgatctgtcagacgtatcttgtggaaaggacgaaacaccg

SEQ ID NO: 32 - DNA, 20 bp, Artificial Sequence
  /note=“Description of Artificial Sequence Synthetic sgRNA sequence”
tggccgtgacatccgaaaga

SEQ ID NO: 33 - DNA, 20 bp, Artificial Sequence
  /note=“Description of Artificial Sequence Synthetic sgRNA sequence”
tcttgatgccaagctcacga

SEQ ID NO: 34 - DNA, 20 bp, Artificial Sequence
  /note=“Description of Artificial Sequence Synthetic sgRNA sequence”
tctttcagcaacacggagta

SEQ ID NO: 35 - DNA, 20 bp, Artificial Sequence
  /note=“Description of Artificial Sequence Synthetic sgRNA sequence”
gcatcttaccatcgtgagct

SEQ ID NO: 36 - DNA, 20 bp, Artificial Sequence
  /note=“Description of Artificial Sequence Synthetic sgRNA sequence”
ttgttgaacctgtttgccta

SEQ ID NO: 37 - DNA, 20 bp, Artificial Sequence
  /note=“Description of Artificial Sequence Synthetic sgRNA sequence”
gtggttctctttgtactggt

SEQ ID NO: 38 - DNA, 20 bp, Artificial Sequence
  /note=“Description of Artificial Sequence Synthetic sgRNA sequence”
gtggtggtaaaatgcacttg

SEQ ID NO: 39 - DNA, 20 bp, Artificial Sequence
  /note=“Description of Artificial Sequence Synthetic sgRNA sequence”
tagtaactccgagccaaatg

SEQ ID NO: 40 - DNA, 20 bp, Artificial Sequence
  /note=“Description of Artificial Sequence Synthetic sgRNA sequence”
ccagttacatcagtcgtgcc

SEQ ID NO: 41 - DNA, 20 bp, Artificial Sequence
  /note=“Description of Artificial Sequence Synthetic sgRNA sequence”
gtggtttaagacaaacacaa

SEQ ID NO: 42 - DNA, 20 bp, Artificial Sequence
  /note=“Description of Artificial Sequence Synthetic sgRNA sequence”
cattgtcgatatgtgcgtgg

SEQ ID NO: 43 - DNA, 20 bp, Artificial Sequence
  /note=“Description of Artificial Sequence Synthetic sgRNA sequence”
ggatagccatctgaattgag

SEQ ID NO: 44 - DNA, 20 bp, Artificial Sequence
  /note=“Description of Artificial Sequence Synthetic sgRNA sequence”
ctaccaacctgcgatcacca

SEQ ID NO: 45 - DNA, 20 bp, Artificial Sequence
  /note=“Description of Artificial Sequence Synthetic sgRNA sequence”
tcgtgtcttggacttcaatg

SEQ ID NO: 46 - DNA, 20 bp, Artificial Sequence
  /note=“Description of Artificial Sequence Synthetic sgRNA sequence”
agtatacgcttgagagacca

SEQ ID NO: 47 - DNA, 20 bp, Artificial Sequence
  /note=“Description of Artificial Sequence Synthetic sgRNA sequence”
agaagctgaccatacactgg

SEQ ID NO: 48 - DNA, 20 bp, Artificial Sequence
  /note=“Description of Artificial Sequence Synthetic sgRNA sequence”
caaccatcaacgctcaccag

SEQ ID NO: 49 - DNA, 20 bp, Artificial Sequence
  /note=“Description of Artificial Sequence Synthetic sgRNA sequence”
gcaggcaaattctgaacttg

SEQ ID NO: 50 - DNA, 20 bp, Artificial Sequence
  /note=“Description of Artificial Sequence Synthetic sgRNA sequence”
atcagcccatttctgcgcac

SEQ ID NO: 51 - DNA, 20 bp, Artificial Sequence
  /note=“Description of Artificial Sequence Synthetic sgRNA sequence”
aggggcagggctatcttatg

SEQ ID NO: 52 - DNA, 20 bp, Artificial Sequence
  /note=“Description of Artificial Sequence Synthetic sgRNA sequence”
gcacatcgttatataccaga

SEQ ID NO: 53 - DNA, 20 bp, Artificial Sequence
  /note=“Description of Artificial Sequence Synthetic sgRNA sequence”
cagggttgcgcagaggactc

SEQ ID NO: 54 - DNA, 20 bp, Artificial Sequence
  /note=“Description of Artificial Sequence Synthetic sgRNA sequence”
gatggcctttgaccgatacg

SEQ ID NO: 55 - DNA, 20 bp, Artificial Sequence
  /note=“Description of Artificial Sequence Synthetic sgRNA sequence”
gatggcatttgacctctctg

SEQ ID NO: 56 - DNA, 20 bp, Artificial Sequence
  /note=“Description of Artificial Sequence Synthetic sgRNA sequence”
gagaaggagatggtttcctg

SEQ ID NO: 57 - DNA, 20 bp, Artificial Sequence
  /note=“Description of Artificial Sequence Synthetic sgRNA sequence”
gatgagatagcactcactgg

SEQ ID NO: 58 - DNA, 20 bp, Artificial Sequence
  /note=“Description of Artificial Sequence Synthetic sgRNA sequence”
gaaaccagcagcccagcatg

SEQ ID NO: 59 - DNA, 20 bp, Artificial Sequence
  /note=“Description of Artificial Sequence Synthetic sgRNA sequence”
ggaggcttcttagatttggg

SEQ ID NO: 60 - DNA, 20 bp, Artificial Sequence
  /note=“Description of Artificial Sequence Synthetic sgRNA sequence”
gagcagcggctgtacaaggt

SEQ ID NO: 61 - DNA, 20 bp, Artificial Sequence
  /note=“Description of Artificial Sequence Synthetic sgRNA sequence”
gcttcaccagtcaaacagca

SEQ ID NO: 62 - DNA, 20 bp, Artificial Sequence
  /note=“Description of Artificial Sequence Synthetic sgRNA sequence”
agatatctatgctttatgtg

SEQ ID NO: 63 - DNA, 20 bp, Artificial Sequence
  /note=“Description of Artificial Sequence Synthetic sgRNA sequence”
cagcatgtgagtaaagtctg

SEQ ID NO: 64 - DNA, 20 bp, Artificial Sequence
  /note=“Description of Artificial Sequence Synthetic sgRNA sequence”
ggaatcctactcagaatgtg

SEQ ID NO: 65 - DNA, 20 bp, Artificial Sequence
  /note=“Description of Artificial Sequence Synthetic sgRNA sequence”
accaatgttcatataaatgg

SEQ ID NO: 66 - DNA, 20 bp, Artificial Sequence
  /note=“Description of Artificial Sequence Synthetic sgRNA sequence”
ctgactgtgttcttagctca

SEQ ID NO: 67 - DNA, 20 bp, Artificial Sequence
  /note=“Description of Artificial Sequence Synthetic sgRNA sequence”
tgaagaggtaatgtctgtcc

(19) United States
(12) Patent Application Publication
(10) Pub. No.: US 2022/0288051 A1
(43) Pub. Date: Sep. 15, 2022

(54) METHODS FOR TREATING CANCER USING SERIAL ADMINISTRATION OF E3 UBIQUITIN LIGASE DEGRADERS

(71) Applicants: Constantine S. MITSIADES, Brookline, MA (US); Ryosuke SHIRASAKI, (US); Geoffrey M. MATTHEWS, Malvern East (AU); Sara GANDOLFI, Carnago (IT); Ricardo DE MATOS SIMOE, Boston, MA (US); Dana-Farber Cancer Institute, Inc., Boston, MA (US)

(72) Inventors: Constantine S. Mitsiades, Brookline, MA (US); Ryosuke Shirasaki, (US); Geoffrey M. Matthews, Malvern East (AU); Sara Gandolfi, Carnago (IT); Ricardo de Matos Simoes, Boston, MA (US)

(21) Appl. No.: 17/641,288

(22) PCT Filed: Sep. 11, 2020
(86) PCT No.: PCT/US20/50339

§ 371 Date: Mar. 8, 2022

Related U.S. Application Data
(60) Provisional application No. 62/899,656, filed on Sep. 12, 2019.

(51) Int. Cl. A61K 31/4545; A61K 31/551; A61K 45/06

(52) U.S. Cl. CPC A61K 31/4545; A61K 31/551; A61K 45/06

(57) ABSTRACT

The present invention relates, in part, to methods for treating cancer using serial administration of E3 ubiquitin ligase degraders.

### CROSS-REFERENCE TO RELATED APPLICATIONS

This application claims the benefit of priority to U.S. Provisional Application Ser. No. 62/899,656, filed on 12 Sep. 2019; the entire contents of said application is incorporated herein in its entirety by this reference.

### STATEMENT OF RIGHTS

This invention was made with government support under Grant RO1 CA050947, CA179483, CA196664, CA180475, U01 CA176058, T32 GM007753, and P30 CA016672-40 awarded by the National Institutes of Health and under Grant W81XWH-15-1-0012 awarded by the Department of Defense. The U.S. government has certain rights in the invention.

### BACKGROUND OF THE INVENTION

The discovery that thalidomide and its immunomodulatory derivatives (IMIDs) recruit the E3 ubiquitin ligase CRBN to induce the ubiquitination and degradation of neomorphic protein substrates (Kronke et al., 2014; Lu et al., 2014) led to the rapid development of “degronimids”, a class of heterobifunctional compounds, in which pairing a thalidomide-like moiety with one of many different small molecular weight agents allows for proteolysis of proteins binding to the latter moieties (Winter et al., 2015). These developments also created new interest in the broader concept of heterobifunctional proteolysis-targeting chimeras (PROTACs): targeting oncogenic proteins for intracellular degradation overcomes several potential limitations related to compounds that merely inhibit their function, including the incomplete and transient target engagement by non-covalent inhibitors; the compensatory increase in levels of the target protein; or the potential oncogenic functions by other uninhibited domains within the protein. Moreover, degronimids and other PROTACs exhibit sub-stoichiometric catalytic activity (Bondeson et al., 2015). Consequently, “degraders” can be designed to incorporate molecules with limited inhibitory potency or, even, agonistic activity, as long as they bind to their target selectively. Given these advantages, degronimids and other pharmacological degraders are being extensively studied in a broad spectrum of malignancies (Lu et al., 2015; Raina et al., 2016; Saenz et al., 2017; Winter et al., 2017). However, the genes or pathways regulating the sensitivity vs. resistance of tumor cells to these agents have not been comprehensively examined. Accordingly, there is a great understanding in the art to determine how overlapping versus distinct these resistance mechanisms are for degraders against different oncoprotein targets and if they involve primarily prevention of, rather than adaptation to, degradation of their respective targets.

### SUMMARY OF THE INVENTION

In one aspect, a method of decreasing the viability of a population of cancer cells comprising contacting the cancer cells with a first heterobifunctional proteolysis-targeting chimera (PROTAC) that recruits an E3 ubiquitin ligase to an oncogenic protein and sequentially contacting the cancer cells with a second heterobifunctional PROTAC that recruits a different E3 ubiquitin ligase to the oncogenic protein, thereby decreasing the viability of the cancer cells, is provided.

In another aspect, a method of delaying or preventing resistance of a population of cancer cells to an anti-oncogenic protein therapy comprising contacting the cancer cells with a first heterobifunctional proteolysis-targeting chimera (PROTAC) that recruits an E3 ubiquitin ligase to an oncogenic protein and sequentially contacting the cancer cells with a second heterobifunctional PROTAC that recruits a different E3 ubiquitin ligase to the oncogenic protein, thereby decreasing the viability of the cancer cells, is provided.

In still another aspect, a method of decreasing the viability of a population of cancer cells previously contacted with a first heterobifunctional proteolysis-targeting chimera (PROTAC) that recruits an E3 ubiquitin ligase to an oncogenic protein comprising contacting the cancer cells with a second heterobifunctional PROTAC that recruits a different E3 ubiquitin ligase to the oncogenic protein, thereby decreasing the viability of the cancer cells, is provided.

In yet another aspect, a method of delaying or preventing resistance of a population of cancer cells to an anti-oncogenic protein therapy comprising contacting the cancer cells with a heterobifunctional PROTAC that recruits an E3 ubiqutin ligase to the oncogenic protein, wherein the cancer cells were previously contacted with a heterobifunctional PROTAC that recruits a different E3 ubiqutin ligase to the oncogenic protein, is provided.

Numerous embodiments are further provided that can be applied to any aspect encompassed by the present invention and/or combined with any other embodiment described herein. For example, in one embodiment, the oncogenic protein is selected from the group consisting of CDK9, BRD2, BRD3, and BRD4. In another embodiment, the E3 ubiquitin ligase is selected from the group consisting of CRBN, VHL, MDM2, APC/C, KCMF1, and RNF4. In still another embodiment, the heterobifunctional PROTAC is selected from the group consisting of dBET6, Thal-SNS-032, ARV-771, and MZ-1. In yet another embodiment, the heterobifunctional PROTAC agents do not contact the cancer cells at the same time. In another embodiment, the at least one additional cancer treatment contacts the cancer cells at the same time as at least one of the heterobifunctional PROTAC agents, such as immunotherapy, targeted therapy, chemotherapy, radiation therapy, hormonal therapy, an anti-cancer vaccine, an anti-cancer virus, a checkpoint inhibitor, radiosensitizer, and combinations thereof. In still another embodiment, the cancer cells are anti-cancer therapy naïve cancer cells. In yet another embodiment, the step of contacting occurs in vivo, ex vivo, or in vitro. In another embodiment, the cancer cells are multiple myeloma cells. In still another embodiment, the cancer cells are in a subject and the subject is an animal model of the cancer. In yet another embodiment, the animal model is a rodent or primate model. In another embodiment, the cancer cells are in a subject and the subject is a mammal. In still another embodiment, the mammal is a rodent, a primate, or a human. In yet another embodiment, the cancer cells and/or subject have undergone cancer treatment, completed treatment, and/or are in remission for the cancer in between contacting the cancer cells with the first heterbifunctional PROTAC agent and the second heterobifunctional PROTAC agent. In another embodiment, the subject has multiple myeloma.

### BRIEF DESCRIPTION OF THE DRAWINGS

FIG. 1A-FIG. 1D show that human MM cells with prior exposure to and tolerance against other agents can remain sensitive to dBET6. Pools of human MM.1S cells transduced with the Cas9 nuclease and a lentiviral genome-scale library (GeCKO v2) of single guide RNAs (sgRNAs) were treated with JQ1 (500 nM) or bortezomib (25 nM), with at least 2-3 rounds of treatment per compound. These drug-exposed populations of MM cells with genome-scale CRISPR-editing were assayed for resistance to these same agents using CTG assays (a-b), to document induced tolerance of these cells to the respective agents. JQ1 or bortezomib-resistant cells were then treated with dBET6 and cell viability, compared with parental-untreated cells, was assessed by CTG (2-way ANOVA analyses; p<0.05 in terms of drug dose and p<0.05 in terms of prior treatment). (c) Cells with prior exposure to and tolerance to JQ1 or bortezomib do not exhibit resistance to dBET6 (2-way ANOVA analyses; p<0.05 in terms of drug dose and p<0.05 in terms of prior treatment with Bort− or JQ1, compared to treatment naive). (d) Bone marrow aspirates obtained from MM patients (and 1 MGUS case) with different disease status and patterns of prior exposure to and resistance/refractoriness to currently available clinical treatments were processed for selection of CD138+ plasma cells. CD138+ cells were then treated with dBET6 or vehicle control. Cell viability was assessed using CTG and individual patient results are shown (RR=relapsed/refractory disease; ND=newly diagnosed disease; MGUS=monoclonal gammopathy of undetermined significance) (Panels a-c depict averages+/−SE of 3 distinct experiments for each panel; panels a-d included triplicates for each condition in each distinct experiment).

FIG. 2A-FIG. 2F show that resistance to dBET6 is mediated by dysregulation of CRBN and its interactors/regulators. (a) Whole-genome CRISPR/Cas9-based gene editing LOF screen led to outgrowth of pools of dBET6-resistant Cas9+MM1S cells. CTG assays confirmed right-shift of the dBET6 dose-response curves compared to dBET6-naive cells (2-way ANOVA analyses; p<0.05 in terms of drug dose and p<0.05 in terms of status of prior treatment or not with dBET6). (b) Next-generation sequencing was performed to quantify the enrichment (average log 2 fold-change of 3 experimental replicates per condition) of sgRNAs for CRBN and several molecules which interact with CRBN, including members of the COP9 signalosome complex. (c-d) MM.1S Cas9+ cells transduced with sgRNAs for individual COP9 signalosome genes (e.g. COPS7B and COPS8; panels c and d, respectively) were tested for their in vitro response to dBET6 (vs. cells transduced with control sgRNAs) (2-way ANOVA analyses; p<0.05 in terms of drug dose and p<0.05 in terms of status of transduction with sgRNAs for COPS7B or COPS8 vs non-targeting control sgRNA). (e) MM cell lines known to express low, but detectable, levels of CRBN, either constitutively (OPM1 and OCI-My5) or after transduction with shRNAs against CRBN (KMS11, OPM2) were tested (compared to control sgRNA transduced cells) for their in vitro response to dBET6. (f) Cas9+MM.1S cells transduced with sgRNAs against CRBN, COPS7B, COPS2, COPS8 (or non-targeting control sgRNA) were tested for their in vitro response to dBET6. (Panels a, c-e depict averages+/−SE of 3 distinct experiments for each panel, with triplicates for each condition in each distinct experiment. Panel f represents averages+/−SE from a single experiment, with triplicates for each condition).

FIG. 3A-FIG. 3F show that resistance to a degronimid against CDK9 (Thal-SNS-032) is mediated by dysregulation of CRBN and its interactors/regulators. We performed a whole-genome CRISPR/Cas9-based gene editing LOF screen to evaluate in a pooled manner which genes are implicated in the development of Thal-SNS-032 resistance by MM.1S cells, as outlined in Materials and Methods, and using an approach similar to the CRISPR screen for resistance to dBET6, i.e., cells received Thal-SNS-032 (25 nM or 15 nM) treatment twice, until an outgrowth of a pool of Thal-SNS-032-resistant cells could be confirmed. (a) CTG assay to determine if this CRISPR screen selected for a pool of Thal-SNS-032-treated (at 4 weeks) MM.1S cells with significantly lower sensitivity to Thal-SNS-032 compared to Thal-SNS-032-naïve cells. (b) Results of next-generation sequencing to quantify in Thal-SNS-032-exposed (at 4 weeks of treatment) MM.1S cells the enrichment (average log 2 fold-change of normalized read counts for 3 experimental replicates per condition) of sgRNAs for CRBN and various molecules which interact with and regulate the function of CRBN, including members of the COP9 signalosome complex. (c-f) MM.1S Cas9+ cells transduced with lentiviral particles for sgRNAs targeting individual COP9 signalosome genes (e.g., COP S7B, COP S2, DDB1, COP S8; panels c through f, respectively) were tested (compared to cells transduced with control sgRNAs) for their responsiveness to Thal-SNS-032 (Panels a, c-f each represent averages+/−SE from triplicates for each experimental condition).

FIG. 4A-FIG. 4C show the results from CRISPR-based gene editing screens for identification of candidate genes associated with decreased response to short-term (48 hrs) or extended treatment with degronimids. (a-b) MM.1S-Cas9+ cells transduced with the Brunello genome-scale library of sgRNAs, were introduced into two CRISPR-based gene editing screens to identify genes associated with decreased response to “short-term” degronimid treatment: unlike the several weeks of successive treatment and outgrowth of cells in the “long-term” screens, the “short-term” screens in panels (a) and (b) involved a single treatment with degronimids (dBET6 25 nM or Thal-SNS-032 25 nM for 48 hrs). At the end of the treatment (48 hrs), tumor cells were collected, processed by Ficoll density centrifugation to isolate live cells; and PCR amplification and next-generation sequencing were performed, similarly to other CRISPR screens in this study, to identify genes with significant differences in their sgRNA content within the tumor cell population at the end of the degronimid treatment. CRBN is the top gene in terms of sgRNA enrichment, but this enrichment is quantitatively less pronounced compared to the results observed in screens with longer-term degronimid treatment. (c) MM.1S-Cas9+ cells, which had developed decreased responsiveness to the CDK9 degrader Thal-SNS-032 in the context of the longterm CRISPR/Cas9 gene editing screen (outlined in FIG. 3), received extended treatment in vitro with either Thal-SNS-032 itself or with dBET6 (i.e., switch from one degronimid to another). After 2 weeks of this extended treatment, live tumor cells were harvested and the distribution of sgRNAs in each of population of cells was quantified by NGS and compared with the distribution of sgRNAs prior to the start of this extended treatment. Panel (c) depicts the average log 2 fold-change (average of 3 experimental replicates per condition) of normalized read counts of sgRNAs after vs. before the extended treatment with each degronimid. Among candidate degronimid resistance genes identified from the “long-term” treatment CRISPR screens, CRBN is the only gene which exhibits enrichment of its sgRNAs after extended degronimid treatment.

FIG. 5A-FIG. 5C show that resistance to VHL-mediated pharmacological degradation of BET bromodomain proteins is mediated by dysregulation of the CUL2-VHL RING ligase complex and its interactors/regulators. We performed whole-genome CRISPR/Cas9-based gene editing LOF screens to evaluate in a pooled manner which genes are implicated in the development of resistance by MM.1S cells against two VHL-mediated pharmacological degraders or BRD2/3/4, namely ARV-771 and MZ-1. Tumor cells received ARV-771 and MZ-1 treatment until an outgrowth of a pool of resistant cells could be confirmed. (a) Average log 2 fold-change (of 3 experimental replicates per condition) of normalized read counts of sgRNAs in ARV-771- or MZ-1-treated MM.1S cells vs. the common vehicle control MM.1S cells. Genes highlighted in red exhibited sgRNA enrichment (3-4 of 4 sgRNAs per gene, p<0.05 [rank aggregation algorithm], log 2FC>1.0) in both screens and are expressed at RPKM>1.0 based on RNA-Seq data on MM.1S cells. (b-c) Cas9+MM.1S cells transduced with sgRNAs against COPS7B, COPS8 (or non-targeting control sgRNA) were tested for their in vitro response to the VHL-based BRD4 degraders MZ-1 (b) and ARV771 (c). (Panels b-c each represent averages+/−SE from a single experiment, with triplicates for each condition).

FIG. 6 shows patterns of essentiality for tumor cells in vitro in drug-free conditions for genes associated with resistance to CRBN- or VHL-mediated pharmacological degraders of oncoproteins. Color-coded heatmaps depict CERES scores, as a quantitative metric of relative dependence of human tumor cell lines, based on CRISPR-based gene editing screens (AVANA sgRNA library) performed in vitro in the absence of drug treatment. CERES scores for MM cell lines are depicted as a matrix (right side of the graph) of cell lines (in columns) and genes (in rows); while for non-MM lines data are depicted for each gene (row) in stacked bar graphs, to visualize the CERES score in descending order (from left to right) each gene. The figure depicts results for genes associated with resistance to degronimids (blue gene symbols), VHL-mediated degraders (red gene symbols) or both, as these genes emerged as top “hits” from standard (i.e., until identification of pools of cells with significant shift-to-the-right for their dose-response curve) CRISPR screens for “degrader” resistance. As indicated in the color-coded scale, black or dark blue color indicates that a given gene has CERES scores compatible with pronounced sgRNA depletion in a given cell line.

FIG. 7A-FIG. 7E show patterns of co-occurrence of essentiality and high transcript levels for known or presumed E3 ligase genes in tumor cell lines. (a-c) Known or presumed E3 ligase genes are examined, based on data from the Achilles Heel CRISPR-based gene editing screens (AVANA sgRNA library; screens performed in vitro in the absence of drug treatment), for the percentage of “highexpressor” cell lines (i.e. cell lines with transcript levels for a given E3 ligase above the average+2SD of a broad range of normal tissues in the GTEx database, similarly to the analyses in FIG. 7b) and the % of “high-expressor” cell lines with CERES scores <−0.5 for that same E3 ligase. Results are depicted for (a) all cell lines of the Achilles Heel CRISPR-based gene editing screens, irrespective of p53 mutational status; (b) p53-mutant cell lines; and (c) p53 wild-type cell lines. Gene symbols in the red or blue boxes represent known or presumed E3 ligases with ≥25% “high-expressor” cell lines & ≥CERES scores <−0.5 in at least ⅔ of “high-expressor” cell lines among p53-mutant cell lines (red box) or all cell lines irrespective of p53 mutational status (blue box). (d) Distribution of CERES scores for essentiality of MDM2 in p53-mutant vs. p53 wild-type cell lines (p<0.0001, t-test). (e) Distribution of CERES scores in MM vs non-MM cell lines for E3 ligases (known or presumed) identified in panels (ac).

FIG. S1A-FIG. S1E show the results of in vitro activity of dBET6 vs. dBET1 or vs. JQ1 against human MM cell lines. (a) CTG assays were performed to quantify the viability of human MM cell lines treated for 72 hrs with different doses of the BET bromodomain degronimids dBET1 and dBET6. Based on these results, dBET6 was selected for the remainder of this study, as a representative of this class of compounds (results represent averages+/−SE from triplicates of each experimental condition). We also compared the effects of dBET6 vs. JQ1 treatment (48 h) on human MM cell lines MIM.1S (b), RPMI-8226 (c) or JJN3 (d) using CTG or CS-BLI. We observed that dBET6 induced more pronounced suppression of MM cell viability than JQ1 (Panels b-d). In a larger panel of human MM cell lines, treatment (48 h) with dBET6 again induced significant loss of viability in all lines tested (panel e) (Panels b-e depict averages+/−SE of 3 distinct experiments for each cell line; with triplicates per condition in each distinct experiment).

FIG. S2A-FIG. S2F show the results of immunoblotting analyses of degronimid-treated MM.1S cells. We treated MM.1S cells with dBET6, Thal-SNS-032 or JQ1 (0.01, 0.05, 0.1, 0.5, 1 μM) or DMSO control for 4 h. Immunoblotting analyses were performed to examine the proteins levels for (a) BRD2; (b) BRD3; (c) BRD4; (d) MYC; and (e-f) CDK9. Each panel represents a different blot, incubated with the antibody for the corresponding target protein and (for panels a-e). GAPDH as a loading control within the same blot. Panel f has been probed with only anti-CDK9 antibody, to highlight more clearly the 2 known CDK9 isoforms, an abundant 42 kDa isoform and a less abundant 55 kDa isoform.

FIG. S3A-FIG. S3C show the results of evaluation of anti-MM activity of dBET6 in co-cultures with bone marrow stromal cells or after exposure to different durations of treatment. (a) CS-BLI viability assay of human luciferase-expressing human MM cell lines cultured alone or with bone marrow stromal cells, and in the presence vs. absence of dBET6, was performed to quantify the dBET6-induced reduction of the percentage of viable tumor cells. (b) Flow cytometry assessment of annexin-V-positive MM.1S cells treated with dBET6 (0.01 μM or 0.05 μM) or vehicle, followed by washout after 4 h, 8 h vs. no washout, to examine whether dBET6 induces a dose- and time-dependent increase in Annexin V+ PI-negative apoptotic cells and Annexin V+PI+ necrotic cells. (c) Dose-response curves, assessed by CS-BLI, on MM.1S cells treated with dBET6 with and without drug washout. Data are reported as the average+/−SE of at least three independent experiments, with at least 3 replicates per experimental condition.

FIG. S4A-FIG. S4C show molecular profiling changes induced in MM cells after pharmacological degradation vs. inhibition of BET bromodomain proteins. (a) Transcriptional profiling changes induced in MM cells with pharmacological degradation vs. inhibition of BET bromodomain proteins: MM1S cells were treated with dBET6 (0.1 μM or 0.5 μM) or JQ1 (0.5 μM) for 4 h, harvested and then processed for RNAseq analysis (n=3 replicates per experimental condition). The heat map depicts the changes in transcriptional profiles induced after treatment with dBET6 vs. JQ1. (b-c) Reverse Phase Protein Array (RPPA) analysis of response to dBET6 in human MM cells: MM.1S cells were treated with dBET6 (0.1 μM) or JQ1 (0.5 μM) and harvested after 4 h or 18 h, followed by RPPA. The heat map of panel b depicts that 4 h and 18 h of dBET6 treatment reduce the expression of key pro-survival proteins, such as MCL-1, c-MYC, and pS6, while leading to the upregulation of cell cycle inhibitor, p21, and cleavage of executioner caspases 3 and 7. The altered expression of key proteins that appear important for the pro-apoptotic effects of dBET6 are shown individually (panel c; n=3 replicates per condition; results depicted as boxplots highlighting the median and min-max range of signal in each experimental condition).

FIG. S5A-FIG. S5D show the results of studies of in vivo anti-tumor activity of dBET6 in two xenograft models of human MM cell lines. Human MM.1S cells were transplanted into NSG mice either on the right flank (subcutaneous model, panels a-b) or via tail vein injection (diffuse model, panels c-d). Treatment with dBET6 (30 mg/kg, QD, 8d) was initiated in the subcutaneous model when tumor size reached approximately 100 mm3, while in the diffuse model, dBET6 treatment (20 mg/kg, QD, 12 days) began 1 week post-tumor inoculation. In the model of subcutaneous lesions, we observed that 8 days of dBET6 treatment significantly suppressed the rate of tumor growth increase (a) and led to a significant increase in overall survival of mice (b; 10d, p<0.05). In the model of diffuse lesions established after intravenous injections of human MM.1S-Luc+ cells, dBET treatment led to significantly lower tumor burden, measured by BLI, compared to vehicle control (c), but only minimal overall survival advantage was detected (d).

FIG. S6 shows a schematic overview of genomewide CRISPR-based functional genomics studies to characterize the mechanisms of tumor cell resistance to CRBN- or VHL mediated pharmacological “degraders” of oncoproteins. We performed genome-scale CRISPR/Cas9 gene editing screens similarly to previous studies (Doench et al., 2016; Meyers et al., 2017; Shalem et al., 2014; Wang et al., 2017) and detailed information included in the Examples. Briefly, MM.1S-Cas9+ cells were transduced with pooled lentiviral particles containing genome-scale sgRNA libraries (GeCKOv2 or Brunello; as indicated in each experiment) and then studied in 3 distinct types of screens, which involved: i) “short-term” (48 hours) treatments with either dBET6 or Thal-SNS-032, followed by tumor cell collection at the end of the treatment; ii) “long-term” studies with successive rounds of dBET6, Thal-SNS-032, ARV-771 or MZ-1 treatment of the pools of MM.1S cells with genome-scale CRISPR-based gene editing, allowing re-growth between treatments and until in vitro drug sensitivity testing confirmed the selection of pools of MM.1S cells with significant shift-to-the-right of their dose-response curve (compared to degrader-naive controls) for the respective treatment; and iii) “extended degronimid treatment” screens, in which Thal-SNS-032-resistant MM.1S Cas9+ cell populations isolated from our initial “long-term” CRISPR/Cas9-based gene editing screen continue receiving additional degronimid treatment for 2 weeks, with either Thal-SNS-032 (i.e., a continuation of the treatment which had led to the isolation of these treatment-resistant cells) or dBET6.

FIG. S7A-FIG. S7D show the results of functional studies to validate the role of individual candidate resistance genes derived from genome-scale CRISPR-based gene editing studies. (a) Cas9+MM.1S cells transduced with sgRNAs against VHL, CRBN (or, serving as controls, sgRNAs against the olfactory receptor genes OR2S2, OR12D2 and OR5AU1) were tested for their in vitro response to ARV771 (left panel) or dBET6 (right panel). (b) Cas9+ KMS-11 cells transduced with sgRNAs against VHL (or, serving as controls, sgRNAs against the olfactory receptor genes OR2H1, OR12D2, OR5V1, OR5AU1 and OR10G2) were tested for their in vitro response to ARV771 (left panel) or dBET6 (right panel). (c) Cas9+MM.1S cells transduced with sgRNAs against TCEB1, TCEB2, CUL2, FBXW2, UBE2R2 (or, as serving as controls, sgRNAs against the olfactory receptor genes OR2H1, OR12D2 and OR5V1) were tested for their in vitro response to ARV771 (left panel) or dBET6 (right panel). (d) Results of insertion/deletion (INDEL) analyses (using the CRISPResso2 algorithm) from “competition” assays in which MM.1S Cas9+ cells transduced with sgRNAs against UBE2R2 or the olfactory receptor (OR) gene OR2S2 were mixed in 1:9 ratio and then cultured in the presence of ARV-771 or DMSO control. The x-axis represents the position (relative to the 3′ of the primer for the sequencing analysis) of each nucleotide of the amplicon containing the UBE2R2 sgRNA cut-site, while the y-axis depicts the fraction of reads with INDELs in each respective position of the amplicon. The ARV771-treated pool of cells with sgUBE2R2 and sgOR (red curve) exhibits higher fraction of INDELs in the UBE2R2 amplicon compared to the control DMSO-treated pool of cells with sgUBE2R2 and sgOR (orange curve); the latter pool had very similar distribution of INDELs as the baseline mixture of cells with sgUBE2R2 and sgOR (black line). The grey curve depicts, as a control, the results for the population of cells with only sgOR.

FIG. S8A-FIG. S8B show the results of sequential administration of CRBN- and VHL-based degraders. (a) Results of initial exposure of tumor cells to CRBN-based degraders followed by VHL-based degraders: Pools of MM cells which had survived CRISPR-based studies after (i) “long-term” treatment with Thal-SNS-032; or “long-term” treatment with Thal-SNS-032 followed by (ii) “extended” treatment with Thal-SNS-032 or (iii) extended treatment with dBET6; vs. populations of drug-naïve cells which remained in culture during the “long-term” or “extended” treatments with these CRBN-based degraders and were collected at the end of the respective studies. Each of these populations (3 replicate pools for each population) were then exposed to dBET6, Thal-SNS-032, ARV-771 or MZ-1 and CTG assays were performed to determine whether pools of MM cells previously exposed to the CRBN-based degraders (dBET6, Thal-SNS-032) exhibited, compared to treatment-naïve cells, major shifts to the right for their dose response curves against these same CRBN-based degraders, but limited, if any, shift for their response to the VHL-based degraders ARV-771 or MZ-1. (b) Results of initial exposure of tumor cells to CRBN-based degraders followed by VHL-based degraders: In a manner similar to panel (a), pools of MM.1S-Cas9+ cells which had survived CRISPR-based studies after “long-term” treatment with ARV771 and then MZ1, were subsequently exposed to dBET6, to determine whether MM cells previously exposed to and tolerant to VHL-based degrader(s) had similar responses, as treatment-naïve cells, to a CRBN-based degrader against the same oncoproteins (BET bromodomain proteins).

FIG. S9A-FIG. S9F show the results of combined administration of CRBN- or VHL-based degraders. MM.1S-Cas9+ cells were exposed simultaneously to the indicated concentrations of (a-b) Thal-SNS-032 plus dBET6; (c-d) Thal-SNS-032 plus ARV-771; and (e-f) dBET6 plus ARV-771. Cell viability was measured by CTG and results are depicted, for each combination, as % of viable cells compared to either drug-free controls (a,c,e) or the respective dBET6- or ARV-771-free cultures for each Thal-SNS-032 or dBET6 dose level (b,d,f).

FIG. S10 show the results of CRISPR-based gene activation studies using genome-scale sgRNA libraries. We performed whole-genome CRISPR/Cas9-based gene activation screens to evaluate in a pooled manner which genes are implicated in the development of resistance by MM.1S cells against (a) dBET6 or (b) ARV-771. For each panel, results depict the average log 2 fold-change (of 3 experimental replicates per condition) of normalized read counts of sgRNAs in dBET6- or ARV-771-treated MM.1S cells vs. the respective vehicle control MM.1S cells. ABCB1 was the only gene with sgRNA enrichment (3-4 of 4 sgRNAs per gene, p<0.05 [rank aggregation algorithm], log 2FC>1.0) in either screen.

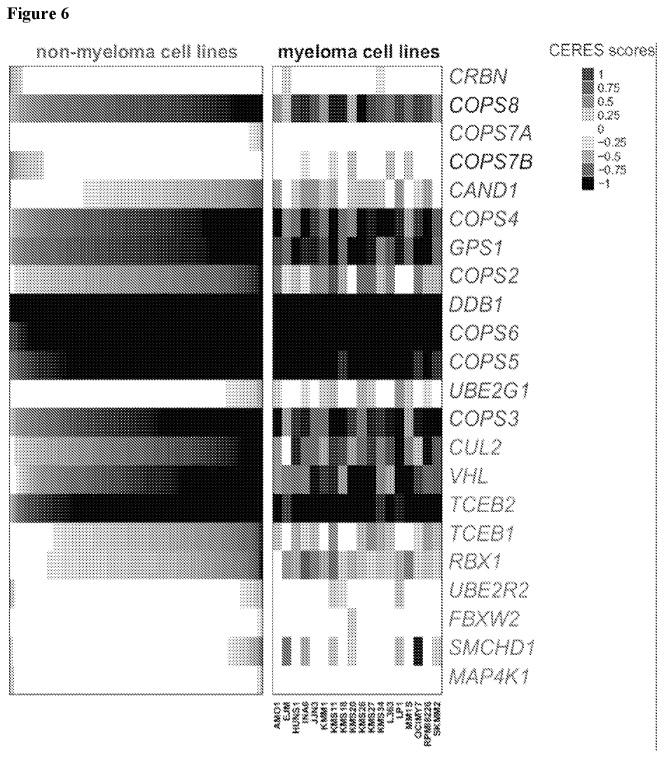

FIG. S11A-FIG. S1113 show the functional genomic landscape of E3 ligases as dependencies in human tumor cells. (a) Color-coded heat-maps depict CERES scores, as a quantitative metric of relative dependence of human tumor cell lines, based on CRISPR-based gene editing screens (AVANA sgRNA library) performed in vitro in the absence of drug treatment. Similarly to FIG. 6, CERES scores for MM cell lines are depicted as a matrix (right side of the graph) of cell lines (in columns) and genes (in rows); while for non-MM lines data are depicted for each gene (row) in stacked bar graphs, to visualize the CERES score in descending order (from left to right) each gene. As indicated in the color-coded scale, black or dark blue color indicates that a given gene has CERES scores compatible with pronounced sgRNA depletion in a given cell line. Panel (a) depicts results for known or presumed E3 ligases, identified based on information from 2 publicly available databases 140.138.144.145/˜ubinet/browseE3.php (Nguyen et al., 2016) and hpcwebapps.cit.nih.gov/ESBL/Database/E3-ligases/ (Medvar et al., 2016). Genes are ranked from top to bottom in descending order of the percentage of all tumor cell lines with CERES scores <−0.2. (b) Example of evaluation of an E3 ligase (in this case MDM2) for the relationship between transcript levels in tumor vs. normal cells and the gene's status as a dependency: Data from the GTEx database were used to define the distribution (average+/−2SD) of MDM2 transcript levels across a broad range of normal tissues. In the bottom panel, transcript levels from matched normal tissues and tumors from the TCGA study; cell lines from the CCLE panel; and GTEx are included for comparative purposes and data are presented in boxplots (representing the average and interquartile range in each group; error bars represent the upper and lower limits of the 95% confidence interval, while individual dots represent samples with outlier expression). The upper part of panel (b) highlights the relationship between transcript levels (based on RNA-Seq data from the CCLE panel) and essentiality (expressed in CERES scores) for MDM2 in tumor cell lines. The bottom right-hand side quadrant of the upper panel highlights tumor cell lines with MDM2 transcript levels above the average+2SD of transcript levels in the normal tissues of the GTEx database (“high expressors”) and CERES scores<−0.5 (consistent with role of MDM2 as an essential gene in the respective cell lines). Linear correlation analysis indicates significant inverse associate of high MDM2 transcript levels and low CERES scores (Spearman correlation coefficient=0.44, p<0.05).

FIG. S12A-FIG. S12B show representative LOF events typically associated with “high-risk”/biologically aggressive MM are not enriched among cells resistant to either CRBN- or VHL-based degraders. Overview of results for key examples of genes whose LOF events are recurrently observed in MM and associated with adverse clinical outcomes (e.g. (Walker et al., 2015)). Depicted results refer to the genome-scale CRISPR-based gene editing LOF screens for resistance to degraders operating through either (a) CRBN (dBET6 and Thal-SNS-032) or (b) VHL (ARV771 and MZ-1).

### DETAILED DESCRIPTION OF THE INVENTION

The present invention is based, at least in part, on the discovery that PROTACs engaging different E3 ligases/CRLs but the same oncoprotein can exhibit antagonism when administered simultaneously, but can overcome cross-resistance when administered sequentially. For example, it is described herein that resistance to “degraders” of different targets (BET bromodomain proteins, CDK9) and operating through CRBN (degronimids) or VHL is primarily mediated by prevention of, rather than adaptation to, breakdown of the target oncoprotein; involves loss-of-function for the cognate E3 ligase or interactors/regulators of the respective cullin-RING ligase (CRL) complex. The substantial gene-level differences for CRBN- vs. VHL-based degraders explains mechanistically the lack of cross-resistance for degraders targeting the same protein via different E3 ligase/CRLs. The results described herein indicate that LOF genetic events (e.g. involving TP53, PTEN) typically associated with high-risk tumors are not enriched among “degrader”-resistant cells, suggesting an important therapeutic opportunity for this class of agents against tumors with prognostically adverse genetic features. The gene-level differences in resistance mechanisms for CRBN- vs. VHL-based degraders may account for the cross-resistance between degraders operating through the same E3 ligase against different oncoproteins, but not for degraders targeting the same protein via different E3 ligase/CRLs. These findings underscore the need to develop new degraders which leverage a broader spectrum of E3 ligases and ideally different CRL complexes; and which can be administered concurrently or sequentially, as an approach to delay or prevent resistance.

Accordingly, the present invention provides a method of decreasing the viability of a population of cancer cells comprising contacting the cancer cells with a first heterobifunctional proteolysis-targeting chimera (PROTAC) that recruits an E3 ubiquitin ligase to an oncogenic protein and sequentially contacting the cancer cells with a second heterobifunctional PROTAC that recruits a different E3 ubiquitin ligase to the oncogenic protein, thereby decreasing the viability of the cancer cells. The present invention further provides a method of delaying or preventing resistance of a population of cancer cells to an anti-oncogenic protein therapy comprising contacting the cancer cells with a first heterobifunctional proteolysis-targeting chimera (PROTAC) that recruits an E3 ubiquitin ligase to an oncogenic protein and sequentially contacting the cancer cells with a second heterobifunctional PROTAC that recruits a different E3 ubiquitin ligase to the oncogenic protein, thereby decreasing the viability of the cancer cells. The present invention also provides a method of decreasing the viability of a population of cancer cells previously contacted with a first heterobifunctional proteolysis-targeting chimera (PROTAC) that recruits an E3 ubiquitin ligase to an oncogenic protein comprising contacting the cancer cells with a second heterobifunctional PROTAC that recruits a different E3 ubiquitin ligase to the oncogenic protein, thereby decreasing the viability of the cancer cells. In addition, the present invention provides a method of delaying or preventing resistance of a population of cancer cells to an anti-oncogenic protein therapy comprising contacting the cancer cells with a heterobifunctional PROTAC that recruits an E3 ubiqutin ligase to the oncogenic protein, wherein the cancer cells were previously contacted with a heterobifunctional PROTAC that recruits a different E3 ubiqutin ligase to the oncogenic protein, is provided. Moreover, the present invention provides additional methods, compositions, kits, and uses described further herein.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a test sample, as compared to the expression level or copy number of the biomarker nucleic acid in a control sample. The term “altered amount” of a biomarker also includes an increased or decreased protein level of a biomarker protein in a sample, e.g., a test sample, as compared to the corresponding protein level in a control sample. Furthermore, an altered amount of a biomarker protein can be determined by detecting posttranslational modification such as methylation status of the marker, which can affect the expression or activity of the biomarker protein.

For the definitions in general (e.g., “altered amount” above, or numerous other definitions below), the “test sample” can be a cancer sample, and the “control sample” can be a normal sample. Alternatively, the “test sample” can be a normal sample (e.g., non-cancer cells), and the “control sample” can be a normal protected sample (e.g., non-cancer cells that have been successfully treated with an agent encompassed by the present invention) or another sample (normal or cancer) that has a known property (e.g., drug refractoriness, low biosynthetic activity, low redox state). These alternative definitions are included, where applicable, in the embodiments that make use of test vs. control type comparisons, even when the embodiments explicitly only refer to one of the two types of alternative definitions of samples (e.g., even when an embodiment only refers to “cancer cells” as the test sample and “normal cells” as the control sample, it can be adaptable to an alternative embodiment in which the test sample has normal cells and the control sample has normal or cancer cells that have been protected or have a known/ascertainable property).

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably by at least 10%, 20%, 30%, 40%, 50%, 60%, 70%, 80%, 90%, 100%, 150%, 200%, 300%, 350%, 400%, 500%, 600%, 700%, 800%, 900%, or 1000% of that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, downregulation, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subject not having the associated disease) and preferably, the average expression level or copy number of the biomarker in several control samples. The altered level of expression is greater or less than the standard error of the assay employed to assess expression or copy number, and is preferably at least 10% 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subject not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker can be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations can be present in the coding or non-coding region of the biomarker nucleic acid.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies can be polyclonal or monoclonal; xenogeneic, allogeneic, or syngeneic; or modified forms thereof (e.g., humanized, chimeric, etc.). Antibodies can also be fully human. Preferably, antibodies encompassed by the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies can also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present invention can include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

An “agonist” is one which enhances, increases, or promotes at least one biological activity and/or the expression levels of at least one biomarker described herein. In certain embodiments, the agonist described herein substantially or completely enhances or promotes a given biological activity and/or the expression levels of at least one biomarker described herein.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation (like in a high proliferation cell), immortality, metastatic potential, rapid growth and proliferation rate, and certain characteristic morphological features. Unless otherwise stated, the terms include metaplasias. As used herein, a “high proliferation cell” refers to a cell that is highly proliferative. Cell proliferation is the process that results in an increase of the number of cells, and can be defined by cell divisions that exceed cell loss through cell death or differentiation. Decreased proliferation can refer to cells in which the number of cell divisions is lower than the number of cell loss. In some embodiments, cell proliferation can be determined by the number of viable cells counted at a first time point and a second time point. For example, if the number of viable cells counted at the second time point is increased relative to the number of viable cells counted at the first time point, then the cells are proliferative. Accordingly, if the level of increase of the number of viable cells of a first cell type is higher than the increase of the number of viable cells of a second cell type, the first cell type has a higher proliferation level than the second cell type. Alternatively, if the level of increase of the number of viable cells of a first cell type is lower than the increase of the number of viable cells of a second cell type, the first cell type has a lower proliferation level than the second cell type. In some embodiments, cell proliferation can be determined using a variety of assays that are known in the art. For example, cell proliferation can be measured by performing DNA synthesis cell proliferation assays, performing metabolic cell proliferation assays, detecting markers of cell proliferation, measuring the concentration of a certain molecule (e.g., intracellular ATP within the cell), and other methods that are known in the art. Those ordinarily skilled in the art will be able to choose a suitable method for determining cell proliferation. In some cases, cell proliferation is high in a cell that, for example, has lost its ability to control its growth. For example, a high proliferation cell can refer to a cancer cell, as described above.

Cancer cells are often in the form of a tumor, but such cells can exist alone within an animal, or can be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, Brenner, or undifferentiated.

In some embodiments, a subject in need thereof has cancer. In some cases, the subject in need thereof that has cancer has a cancer that is caused by a virus. Similarly, in some embodiments, cancer cells include cells obtained from a cancer that has been caused by viral infection. Both DNA and RNA viruses have been shown to be capable of causing cancer. DNA viruses that are known to cause cancer include, without limitation, Epstein-Barr virus (EBV), human papilloma virus (HPV), hepatitis B virus (HBV), Merkel cell polyomavirus (MCV) and human herpes virus-8 (HHV-8). RNA viruses that are known to cause cancer include, without limitation, human T-lymphotrophic virus-1 (HTLV-15 1) and hepatitis C virus (HCV). In some embodiments, cancer cells include cells obtained from a cancer that has been caused by a bacterial infection. For example, Helicobacter pylori and Chalmydia trachomatis are known to cause cancer. In some embodiments, cancer cells include cells obtained from a cancer that has been caused by a parasite. For example, Opisthorchis viverrini, Clonorchis sinensis and Schistosoma haematobium are parasites known to cause cancer.

In some embodiments, a high proliferation cell is a cancer cell that is derived from a biological sample from a subject. For example, a high proliferation cell useful in the methods of the present disclosure can be a human cancer cell derived from a biological sample from a human having cancer.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample can comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control can comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control can comprise normal or non-cancerous cell/tissue sample. In another preferred embodiment, the control can comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control can comprise normal cells, cells from patients treated with combination chemotherapy, and cells from patients having benign cancer. In another embodiment, the control can also comprise a measured value for example, average level of expression of a particular gene in a population compared to the level of expression of a housekeeping gene in the same population. Such a population can comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control can comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods encompassed by the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code encompassed by the present invention to determine the presence or absence of a cancer or subtype thereof in an individual. The term also includes methods, systems, and code for assessing the level of disease activity in an individual.

A molecule is “fixed” or “affixed” to a substrate if it is covalently or non-covalently associated with the substrate such that the substrate can be rinsed with a fluid (e.g., standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature can be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “immunotherapy” or “immunotherapies” refer to any treatment that uses certain parts of a subject's immune system to fight diseases such as cancer. The subject's own immune system is stimulated (or suppressed), with or without administration of one or more agents for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulate immune system function. Representative examples of untargeted therapies include, without limitation, chemotherapy, gene therapy, and radiation therapy.

Immunotherapy is one form of targeted therapy that can comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They can also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013) Annu. Rev. Immunol. 31:51-72). Specific representative examples of consensus immunogenic chemotherapies include anthracyclines, such as doxorubicin and the platinum drug, oxaliplatin, 5′-fluorouracil, among others.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragments, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773). As a reprentative of activity of immune checkpoints in general, the term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response. Similarly, as a reprentative example of any immune checkpoint, immune checkpoint ligands are well known such as “PD-1 ligand,” which refers to binding partners of the PD-1 receptor and includes both PD-L1 (Freeman et al. (2000) J. Exp. Med. 192:1027-1034) and PD-L2 (Latchman et al. (2001) Nat. Immunol. 2:261).

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can bind to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “oncogenic protein” refers to a protein that cell cycle progression and/or cellular transformation and are well-known in the art. The activity of oncogenice proteins can be modulated (e.g., downregulated) though targeting not only the protein, but many other molecules or events that participate in the same pathways as the oncogenic protein, such as the ubiquitinylation pathway as described further herein.

The term “activity” includes the ability of an agent like a PROTAC or polypeptide (and its fragments, domains, and/or motifs thereof, discussed herein) to bind other proteins and to regulate signaling pathways (as described herein) in a cell (e.g., a cancer cell, and/or an immune cell).

The term “substrate(s)” refers to binding partners of an agent like a PROTAC or polypeptide (and its fragments, domains, and/or motifs thereof, discussed herein), e.g., the proteins described herein and/or known by a skilled artisan.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein. The term “anti-cancer agent” or “cancer therapeutic agent” includes immunotherapeutic agents.

The term “agent” or “therapeutic agent” when used in the context of reducing cytotoxicity or side effects associated with cancer treatment can include any molecule, peptide, antibody or other agent encompassed by the present invention in cells of a biological material or a subject. In that context (i.e., the context of reducing cytotoxicity or side effects associated with cancer treatment), the terms “agent,” “therapeutic agent,” and “protective agent” are used interchangeably. In addition, usage of protective agents can be referred to as protective therapy. Various agents are useful in the compositions and methods described herein. For example, the protective agent can include 7-nitro-N-(2-phenylphenyl)-2,1,3-benzoxadiazol-4-amine; thioxothiazolidinone [Z-]-5-[4-ethylbenzylidene]-2-thioxo-1,3-thiazolidin-4-one; 4-phenylbutyrate; Compound 0012; curcumin; magnesium hydroxide; BP-1-102; WP1 193; BP-1-107; BP-1-108; SF-1-087; SF-1-088; STX-0119; substituted thiazol-4-one compounds; (Z,E)-5-(4-ethylbenzylidene)-2-thioxothiazolidin-4-one; S2T1-60TD; quarfloxin; benzoylanthranilic acid; cationic porphyrin TMPyP4; tyrphostin, tryphostin-like compounds; AG490; FBXW-7 expression vectors; JQ1; dBET6; MZ-1; ARV-771; BAY1238097; BMS-986158; CPI-0610; FT-1101; GS-5829; GSK2820151; GSK525762; INCB054329; R06870810; ODM-207; AZD5153; OTX015; CPI203; ZEN003694; INCB054329; MK-8628; BMS-986158; AZD1775; RVX000222; LY294002; and combinations thereof.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies of the disclosed invention can be measured or determined by standard antibody-antigen assays, for example, competitive assays, saturation assays, or standard immunoassays such as ELISA or RIA.

A “kit” is any manufacture (e.g., a package or container) comprising at least one reagent, e.g., a probe or small molecule, for specifically detecting and/or affecting the expression of a marker of the present invention. The kit can be promoted, distributed, or sold as a unit for performing the methods of the present invention. The kit can comprise one or more reagents necessary to express a composition useful in the methods of the present invention. In certain embodiments, the kit can further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit can be provided in individual containers or as mixtures of two or more reagents in a single container. In addition, instructional materials which describe the use of the compositions within the kit can be included.

The term “mode of administration” includes any approach of contacting a desired target with a desired agent, including providing biophysical agents for chemotherapy and also including providing radiation for radiotherapy, that is used to contact cells, such as contacting cancer cells and/or non-cancer cells. The route of administration, as used herein, is a particular form of the mode of administration, and it specifically covers the routes by which biophysical agents are administered to a subject or by which biophysical agents are contacted with a biological material.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker amount and/or activity measurement(s) can be a biomarker amount and/or activity measurement(s) used to, by way of example only, evaluate a subject that can be selected for a particular treatment, evaluate a response to a treatment such as encompassed by the present invention, either alone or in combination with a cancer therapy such as cytotoxic chemotherapy, radiotherapy, and/or an immunotherapy like an immune checkpoint inhibitor. A pre-determined biomarker amount and/or activity measurement(s) can be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject can affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cell to agents encompassed by the present invention, either alone or in combination with a cancer therapy such as cytotoxic chemotherapy, radiotherapy, and/or an immunotherapy like an immune checkpoint inhibitor. Such predictive use of the biomarker can be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g., a human, afflicted with cancer; (3) its absolute or relatively modulated presence or absence in clinical subset of patients with cancer (e.g., those responding to a particular agent encompassed by the present invention, either alone or in combination with a cancer therapy such as cytotoxic chemotherapy, radiotherapy, and/or an immunotherapy like an immune checkpoint inhibitor or those developing resistance thereto).

The term “pre-malignant lesions” as described herein refers to a lesion that, while not cancerous, has potential for becoming cancerous. It also includes the term “pre-malignant disorders” or “potentially malignant disorders.” In particular this refers to a benign, morphologically and/or histologically altered tissue that has a greater than normal risk of malignant transformation, and a disease or a patient's habit that does not necessarily alter the clinical appearance of local tissue but is associated with a greater than normal risk of precancerous lesion or cancer development in that tissue (leukoplakia, erythroplakia, erytroleukoplakia lichen planus (lichenoid reaction) and any lesion or an area which histological examination showed atypia of cells or dysplasia. In one embodiment, a metaplasia is a pre-malignant lesion.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The term “response to inhibitor or therapy” relates to any response of the hyperproliferative disorder (e.g., cancer) to a therapeutic agent (e.g., a protective agent that reduces side effects or cytotoxicity from cancer treatment), either alone or in combination with a cancer therapy such as cytotoxic chemotherapy, radiotherapy, and/or an immunotherapy like an immune checkpoint inhibitor, preferably to a change in tumor mass and/or volume after initiation of neoadjuvant or adjuvant therapy. Hyperproliferative disorder response can be assessed, for example for efficacy or in a neoadjuvant or adjuvant situation, where the size of a tumor after systemic intervention can be compared to the initial size and dimensions as measured by CT, PET, mammogram, ultrasound or palpation. Responses can also be assessed by caliper measurement or pathological examination of the tumor after biopsy or surgical resection. Response can be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response can be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement can be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects can be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing logarithmically.

The terms “response” or “responsiveness” refers to an anti-cancer response, e.g., in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance can also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “substantial contact” or “substantially contact” refers to a degree of association between at least two objects that is at least sufficient to allow the two objects to interact with each other. For example, for a single cell and a single ligand, there is substantial contact if the ligand occupies an appropriate receptor on the cell or if the ligand is inside the cell. As another example, for a single cell and a population of ligands with a certain concentration, there is substantial contact if a certain fraction of the ligands occupies an appropriate receptor on the cell or if a certain fraction of the ligands is inside the cell. The fraction that would give rise to a substantial contact, in an embodiment is 60%. In other embodiments, the fraction that would give rise to substantial contact is 65%, 70%, 75%, 80%, 85%, 90%, or 95%. In contrast, a fraction below a certain value, such as 20%, would constitute a “lack of substantial contact,” implying that the population of ligands does “not substantially contact” the cell. For a lack of substantial contact, the fraction can be 15%, 10%, 5%, or 1%.

The term “survival” includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more therapeutic agents (e.g., at least two heterobifunctional PROTACs, or at least one heterobifunctional PROTAC combined with a cancer therapy, such as immunotherapy like an immune checkpoint inhibitor) can be greater (i.e., better, as in less undesirable effects and more desirable effects) than the sum of the separate effects of the agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. Therapeutic effect also includes reduction of undesirable cytotoxicity in cells, for example those associated with cancer treatment. In addition, therapeutic effect includes reduction of undesirable side effects in subjects, for example those associated with cancer treatment. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods encompassed by the present invention can be administered in a sufficient amount to produce a reasonable benefit/risk ratio applicable to such treatment. The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds can be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). The code below is the standard one, but one can use other known codes (e.g., for mitochondria) as well. Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet can be employed (illustrated above). Therefore, a number of different nucleotide sequences can code for a given amino acid sequence. Such nucleotide sequences are considered functionally equivalent since they result in the production of the same amino acid sequence in all organisms (although certain organisms can translate some sequences more efficiently than they do others). Moreover, occasionally, a methylated variant of a purine or pyrimidine can be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers encompassed by the present invention are well-known in the art and readily available on publicly available databases, such as the National Center for Biotechnology Information (NCBI). For example, exemplary nucleic acid and amino acid sequences derived from publicly available sequence databases are provided in the Examples below and are well-known in the art.

### II. Heterobifunctional Proteolysis-Targeting Chimeras (PROTACs)

Heterobifunctional proteolysis-targeting chimeras (PROTACs) represent a class of agents that allow for proteolysis of proteins of interest, such as oncogenic proteins, by recruiting the protein to an E3 ubiquitin ligase for degradation via the proteasome. Numerous E3 ubiquitin ligases and oncogenic proteins are well-known in the art and further described in the Examples below.

For example, in one embodiment, the description provides compounds comprising an E3 ubiquitin ligase binding moiety (“ULM”) that is an IAP E3 ubiquitin ligase binding moiety (an “ILM”), a cereblon E3 ubiquitin ligase binding moiety (a “CLM”), a Von Hippel-Lindau E3 ubiquitin ligase (VHL) binding moiety (VLM), and/or a mouse double minute 2 homologue (MDM2) E3 ubiquitin ligase binding moiety (MLM). Detailed descriptions of representative structures described below can be applied to any E3 ubiquitin ligase and binding moiety thereof of a PROTAC of interest.

In an exemplary embodiment, the ULM is coupled to a target protein binding moiety (PTM) via a chemical linker (L). As would be understood by the skilled artisan, compounds described herein can be synthesized with any desired number and/or relative position of the respective functional moieties.

The terms ULM, ILM, VLM, MLM, and CLM are used in their inclusive sense unless the context indicates otherwise. For example, the term ULM is inclusive of all ULMs, including those that bind IAP (i.e., ILMs), MDM2 (i.e., MLM), cereblon (i.e., CLM), and VHL (i.e., VLM). Further, the term ILM is inclusive of all possible IAP E3 ubiquitin ligase binding moieties, the term MLM is inclusive of all possible MDM2 E3 ubiquitin ligase binding moieties, the term VLM is inclusive of all possible VHL binding moieties, and the term CLM is inclusive of all cereblon binding moieties.

In another embodiment, the present disclosure provides bifunctional or multifunctional compounds (e.g., PROTACs) useful for regulating protein activity by inducing the degradation of a target protein. In certain embodiments, the compound comprises an ILM or a VLM or a CLM or a MLM coupled, e.g., linked covalently, directly or indirectly, to a moiety that binds a target protein (i.e., a protein targeting moiety or a “PTM”). In certain embodiments, the ILM/VLM/CLM/MLM and PTM are joined or coupled via a chemical linker (L). The ILM binds the IAP E3 ubiquitin ligase, the VLM binds VHL, CLM binds the cereblon E3 ubiquitin ligase, and MLM binds the MDM2 E3 ubiquitin ligase, and the PTM recognizes a target protein and the interaction of the respective moieties with their targets facilitates the degradation of the target protein by placing the target protein in proximity to the ubiquitin ligase protein. In certain embodiments, the ULM (e.g., a ILM, a CLM, a VLM, or a MLM) shows activity or binds to the E3 ubiquitin ligase (e.g., IAP E3 ubiquitin ligase, cereblon E3 ubiquitin ligase, VHL, or MDM2 E3 ubiquitin ligase) with an IC.sub.50 of less than about 200 M. The IC.sub.50 can be determined according to any method known in the art, e.g., a fluorescent polarization assay.

In certain additional embodiments, the bifunctional compounds described herein demonstrate an activity with an IC₅₀ of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 M, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 pM.

In certain embodiments, the compounds as described herein comprise multiple PTMs (targeting the same or different protein targets), multiple ULMs, one or more ULMs (i.e., moieties that bind specifically to multiple/different E3 ubiquitin ligase, e.g., VHL, IAP, cereblon, and/or MDM2) or a combination thereof. In any of the embodiments or embodiments described herein, the PTMs and ULMs (e.g., ILM, VLM, CLM, and/or MLM) can be coupled directly or via one or more chemical linkers or a combination thereof. In additional embodiments, where a compound has multiple ULMs, the ULMs can be for the same E3 ubiquintin ligase or each respective ULM can bind specifically to a different E3 ubiquitin ligase. In still further embodiments, where a compound has multiple PTMs, the PTMs can bind the same target protein or each respective PTM can bind specifically to a different target protein.

In certain embodiments, where the compound includes multiple ULMs, the ULMs are identical. In additional embodiments, the compound including a plurality of ULMs (e.g., ULM, ULM′, etc.), at least one PTM coupled to a ULM directly or via a chemical linker (L) or both. In certain additional embodiments, the compound including a plurality of ULMs further includes multiple PTMs. In still additional embodiments, the PTMs are the same or, optionally, different.

In still further embodiments, wherein the PTMs are different, the respective PTMs may bind the same protein target or bind specifically to a different protein target.

In certain embodiments, the compound may comprise a plurality of ULMs and/or a plurality of ULM's. In further embodiments, the compound comprising at least two different ULMs, a plurality of ULMs, and/or a plurality of ULM's further comprises at least one PTM coupled to a ULM or a ULM′ directly or via a chemical linker or both. In any of the embodiments described herein, a compound comprising at least two different ULMs can further comprise multiple PTMs. In still additional embodiments, the PTMs are the same or, optionally, different. In still further embodiments, wherein the PTMs are different the respective PTMs may bind the same protein target or bind specifically to a different protein target. In still further embodiments, the PTM itself is a ULM (or ULM), such as an ILM, a VLM, a CLM, a MLM, an ILM′, a VLM′, a CLM′, and/or a MLM′.

In additional embodiments, the description provides the compounds as described herein including their enantiomers, diastereomers, solvates and polymorphs, including pharmaceutically acceptable salt forms thereof, e.g., acid and base salt forms.

In some embodiments, “degronoimids” are included as a sub-genus of heterobifunctional PROTACs and such degronimids are well-known in the art as described further in the Examples.

Moreover, representative examples of heterobifunctional PROTACs and their structures are well-known in the art (see, for example, U.S. Patent Application Publications Nos. 2015/0291562, 2014/0356322, 2019/0276459) and publications in the Examples.

### III. Subjects

In one embodiment, a subject is a mammal (e.g., mouse, rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer. In addition, cells can be used according to the methods described herein, whether in vitro, ex vivo, or in vivo, such as cells from such subjects.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue can be located in an inoperable region of the body, such as in a tissue that is essential for life, or in a region where a surgical procedure would cause considerable risk of harm to the patient.

The methods encompassed by the present invention can be used across many different cancers in subjects such as those described herein.

### IV. Therapeutic Methods

A protective therapy, according to some embodiments of the invention, includes administering agents (e.g., heterobifunctional PROTAC) to contact cancer cells, such as in a subject. The cancer cells are contacted with the agents in a temporally uncoupled fashion, for example by administering the agents sequentially rather than concomitantly. In some embodiments, the sequence contact means that the cancer cells are not put into contact with the agents at the same time. This process can also occur in the context of additional cancer therapies, such as contactin the cancer cells with the agents before initiating cancer treatment, or by administering the agents during or after cancer treatment.

The order and combination of therapies/treatments can be varied. For example, therapy can be before, during, or after the cancer treatment, and therapy can be combined with one or more other anti-cancer treatments. Various combinations can have synergy. For example, when a heterobifunctional PROTAC and another heterobifunctional PROTAC are administered sequentially, it can be possible to use higher doses/dosages of the heterobifunctional PROTAC agents or other anti-cancer agents as compared to the doses/dosages that would have been permissible without the sequential administration.

The term “targeted therapy” refers to administration of agents that selectively interact with a chosen biomolecule to thereby treat cancer. One example includes immunotherapies such as immune checkpoint inhibitors, which are well-known in the art. For example, anti-PD-1 pathway agents, such as therapeutic monoclonal blocking antibodies, which are well-known in the art and described above, can be used to target tumor microenvironments and cells expressing unwanted components of the PD-1 pathway, such as PD-1, PD-L1, and/or PD-L2.

Immunotherapies that are designed to elicit or amplify an immune response are referred to as “activation immunotherapies.” Immunotherapies that are designed to reduce or suppress an immune response are referred to as “suppression immunotherapies.” In some embodiments, immunotherapy can be “untargeted,” which refers to administration of agents that do not selectively interact with immune system cells, yet modulates immune system function.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (MET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still another embodiment, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet another embodiment, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with agents encompassed by the present invention, alone or in combination with an immunotherapy, to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent can be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) can also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1, 8-naphthalimide; (Trevigen); 6(5H)-phenanthridinone (Trevigen); benzamide (U.S. Pat. No. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat can help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area can be heated externally with high-frequency waves aimed at a tumor from a device outside the body. To achieve internal heating, one of several types of sterile probes can be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It can also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and can be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time can be needed because the precision of the laser allows for a smaller incision. Healing time is often shortened; since laser heat seals blood vessels, there is less bleeding, swelling, or scarring. Laser surgery can be less complicated. For example, with fiber optics, laser light can be directed to parts of the body without making a large incision. More procedures can be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They can be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also can be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers can be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat can help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies can vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods encompassed by the present invention is a factor in determining optimal treatment doses and schedules.

Any means for the introduction of a polynucleotide into mammals, human or non-human, or cells thereof can be adapted to the practice of this invention for the delivery of the various constructs encompassed by the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs can first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g., with various lipid or liposome materials, can then be effected using known methods and materials and delivered to the recipient mammal. See, e.g., Canonico et al. Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system can be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest can be in a plasmid or viral vector, or other vector as is known in the art. Such vectors are well-known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter can be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide encompassed by the present invention have been derived from herpes virus, e.g., Herpes Simplex Virus (U.S. Pat. No. 5,631,236 by Woo et al., issued Can 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, can be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### VII. Clinical Efficacy

Clinical efficacy can be measured by any method known in the art. For example, the benefit from a therapy, alone or in combination with a cancer therapy such as cytotoxic chemotherapy, radiotherapy, and/or an immunotherapy like an immune checkpoint inhibitor, relates to a change in the cytotoxicity, and can also relate to any response of the cancer, e.g., to a change in tumor mass and/or volume after initiation of therapy, such as neoadjuvant or adjuvant cytotoxic chemotherapy. Tumor response can be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response can also be assessed by caliper measurement or pathological examination of the tumor after biopsy or surgical resection. Response can be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response can be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

The benefit from using agents encompassed by the present invention can be determined by measuring the level of cytotoxicity in a biological material. The benefit from using agents encompassed by the present invention can be assessed by measuring transcription profiles, viability curves, microscopic images, biosynthetic activity levels, redox levels, and the like. The benefit from using agents encompassed by the present invention can also be determined by measuring the amount of side effects from the cancer treatment.

In some embodiments, the ability of an agent encompassed by the present invention to reduce cellular ciability can be measured by assessing cell proliferation, which can be determined by the number of viable cells counted at a first time point and a second time point. For example, if the number of viable cells counted increased less, remained unchanged, or decreased between a first time point and a second time point in a sample contacted with a test agent as compared to a sample contacted with a control agent, then the test agent can decrease cell proliferation.

In some embodiments, cell proliferation can be determined using a variety of assays that are known in the art. For example, cell proliferation can be measured by performing DNA synthesis cell proliferation assays, performing metabolic cell proliferation assays, detecting markers of cell proliferation, measuring the concentration of a certain molecule (e.g., intracellular ATP within the cell), and other methods that are known in the art. Those ordinarily skilled in the art will be able to choose a suitable method for determining cell proliferation.

In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to immunotherapies, such as anti-immune checkpoint therapies, are related to “survival,” which includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any immunotherapy, such as anti-immune checkpoint therapy. The outcome measurement can be pathologic response to therapy given in the neoadjuvant setting. Alternatively, outcome measures, such as overall survival and disease-free survival can be monitored over a period of time for subjects following immunotherapies for whom biomarker measurement values are known. In certain embodiments, the same doses of immunotherapy agents, if any, are administered to each subject. In related embodiments, the doses administered are standard doses known in the art for those agents used in immunotherapies. The period of time for which subjects are monitored can vary. For example, subjects can be monitored for at least 2, 4, 6, 8, 10, 12, 14, 16, 18, 20, 25, 30, 35, 40, 45, 50, 55, or 60 months. Biomarker measurement threshold values that correlate to outcome of an immunotherapy can be determined using methods such as those described in the Examples section.

### V. Administration of Agents

The immune modulating agents encompassed by the present invention are administered to subjects in a biologically compatible form suitable for pharmaceutical administration in vivo, to enhance immune cell mediated immune responses. By “biologically compatible form suitable for administration in vivo” is meant a form to be administered in which any toxic effects are outweighed by the therapeutic effects. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition encompassed by the present invention is defined as an amount effective, at dosages and for periods of time necessary, to achieve the desired result. For example, a therapeutically active amount of an agent can vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

Agents encompassed by the present invention can be administered either alone or in combination with an additional cancer therapy. In the combination therapy, encompassed by the present invention and anti-cancer agents can be delivered to different cells and can be delivered at different times. The agents encompassed by the present invention can be incorporated into pharmaceutical compositions suitable for administration. Such compositions can comprise the nucleic acid molecule, protein, antibody, modulatory compound, or modulatory molecule and a pharmaceutically acceptable carrier.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which can inactivate the compound. For example, for administration of agents, by other than parenteral administration, it can be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, nonionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions encompassed by the present invention can be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intra-vaginally or intra-rectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity, or expression and/or activity of the complex, or composition comprising an agent that modulates (e.g., inhibits) biomarker expression and/or activity, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods encompassed by the present invention can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods encompassed by the present invention include those suitable for oral, nasal, topical (including buccal and sublingual), rectal, vaginal, aerosol and/or parenteral administration. The formulations can conveniently be presented in unit dosage form and can be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound can also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered peptide or peptidomimetic moistened with an inert liquid diluent.

Tablets, and other solid dosage forms, such as dragees, capsules, pills and granules, can optionally be scored or prepared with coatings and shells, such as enteric coatings and other coatings well-known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions, which can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration can be presented as a suppository, which can be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component can be mixed under sterile conditions with a pharmaceutically-acceptable carrier, and with any preservatives, buffers, or propellants which can be required.

The ointments, pastes, creams and gels can contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

Examples of suitable aqueous and nonaqueous carriers which can be employed in the pharmaceutical compositions encompassed by the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents encompassed by the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention can be determined by the methods encompassed by the present invention so as to obtain an amount of the active ingredient, which is effective to achieve the desired therapeutic response for a particular subject, composition, and mode of administration, without being toxic to the subject.

The nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment can increase or decrease over the course of a particular treatment. Changes in dosage can result from the results of diagnostic assays.

### VI. Kits

The present invention also encompasses kits comprising agents encompassed by the present invention. A kit encompassed by the present invention can also include instructional materials disclosing or describing the use of the kit or an antibody of the disclosed invention in a method of the disclosed invention as provided herein. A kit can also include additional components to facilitate the particular application for which the kit is designed. For example, a kit can additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit can additionally include buffers and other reagents recognized for use in a method of the disclosed invention. Non-limiting examples include agents to reduce non-specific binding, such as a carrier protein or a detergent.

### EXAMPLES

### Example 1: Materials and Methods for Examples 2-9

a. Summary

We examined the biological responses of human MM cells to the degronimids dBET6 (Winter et al., 2017) and Thal-SNS-032 (Olson et al., 2018), as well as the VHL-mediated degraders of BRD4/3/2, ARV771 and MZ-1, using protocols similar to those described in our prior studies on drug sensitivity testing assays, such as CTG (Delmore et al., 2011) or CS-BLI for tumor cell monoculture vs. co-culture with BMSCs (McMillin et al., 2012a; McMillin et al., 2010); Annexin V/PI staining for assessment of apoptotic cell death (Delmore et al., 2011); RNA-sequencing (Wan et al., 2017), immunoblotting (Matthews et al., 2015; Newbold et al., 2013), reverse phase protein array (RPPA) studies (Li et al., 2017); as well as in vivo efficacy studies of dBET6 treatment of xenografts established in NSG mice after subcutaneous (Delmore et al., 2011; McMillin et al., 2011; McMillin et al., 2010; McMillin et al., 2012b; Mitsiades et al., 2008) or intravenous (Delmore et al., 2011; Shalem et al., 2014) injection of human MM.1S cells. More detailed information on these experimental procedures, as well as the design of our genome-scale CRISPR-based gene editing screens for genes whose loss of function confers resistance to CRBN- or VHL-mediated degraders, is provided below.

b. Cell Culture

The human cell lines (MM.1S, RPMI-8226, OPM-2, OPM-1, JJN3, L363, AMO-1, OCI-My5) were obtained from ATCC or DSMZ. OPM-2.shRNA. CRBN and KMS11.shRNA. CRBN cells were a gift from Keith Stewart (Mayo Clinic, AZ, USA) CRBN−/− cells were kindly provided by the laboratory of Dr William Kaelin (DFCI, Boston, Mass., USA). The MM.1S-Cas9 cell line was generated by the laboratory of Dr Benjamin Ebert (DFCI). Human stromal cell line HS27A was obtained from ATCC. All human MM cell lines were cultured in RPMI 1640 medium supplemented with L-glutamine (Life Technologies, Carlsbad, Calif., USA), FBS (10%) (Gemini Bioproducts, Woodland, Calif.), 20 I.U./mL penicillin and 20 μg/mL streptomycin (Fisher Scientific, Springfield, N.J., USA) and cultured at 37° C., with 5% CO₂.

c. Patient-Derived Samples

Bone marrow aspirates (1-4 mL) from individuals with MM (newly diagnosed, smoldering, relapsed/refractory, maintenance treatment) or MGUS were collected after patients provided informed consent and based on tissue collection protocol approved by the Dana-Farber Cancer Institute Institutional Review Board. Samples were processed for separation of CD138+ plasma cells, using CD138-positive selection beads, and immunomagnetic separation, as per kit instructions (EasySep, StemCell, Cambridge, Mass.). Patient-derived tumor cells were cultured in in RPMI 1640 medium supplemented with L-glutamine (Life Technologies, Carlsbad, Calif., USA), FBS (10%) (Gemini Bioproducts, Woodland, Calif.), 20 I.U./mL penicillin and 20 μg/mL streptomycin (Fisher Scientific, Springfield, N.J., USA) and hIL-6 (Thermo Fisher Scientific, Waltham, Mass., USA 1-5 μg/mL).

d. Mice

NOD. Cg-Prkdcscid Il2rgtm1Wjl/SzJ (NSG) female mice were purchased from Jackson Laboratory. NSG mice were previously described (Shultz L D et al., 2005). Mice were bred and maintained in individual ventilated cages and fed with autoclaved food and water at Dana-Farber Animal Facility. Animal studies were performed according to a protocol approved by the Dana-Farber Cancer Institute Animal Care and Use Committee.

e. Immunoblotting

MM.1S cells were seeded in 6-well plates at 3×10⁶ cells/well (˜24 h prior to addition of drug). After the addition of dBET6, JQ1 or Thal-SNS-032 cells were incubated, harvested after 4 h using trypsin, washed in ice-cold PBS and immediately frozen (−80° C.). Cell pellets were thawed on ice, lysed using RIPA buffer (ThermoFisher) with protease/phosphatase inhibitor cocktail (Cell Signal Technology, Danvers, Mass.) by incubating on ice 30 min. The lysates were clarified by spinning at 10,000 g for 10 min at 4° C. and the concentration of the lysate was determined using BCA protocol (ThermoFisher). Samples were prepared for Western blot using LDS sample buffer (NuPage, Invitrogen, Carlsbad, Calif., USA) with sample reducing agent (NuPage, Invitrogen) and heated to 95° C. for 5 min. Samples were loaded (20 ug/sample) into Tris-acetate or Bis-Tris gels (NuPage, Invitrogen) and run as per kit instructions using appropriate running buffers. Gels were transferred as described (Matthews et al., 2015) onto PVDF membrane using SDS-based transfer buffer (NuPage, Invitrogen). Membranes were blocked (5% skim milk; or 5% bovine serum albumin in TBS-T) for at least 30 min then probed with primary antibodies overnight (4° C.). Secondary antibodies were incubated for 1 h at RT (5% skim milk in TBS-T) prior to incubation in ECL (ThermoFisher). Each protein of interest (BRD2, BRD3, BRD4, c-myc and CDK9) was assessed in a separate gel and corresponding membrane (which was simultaneously incubated with antibodies for both the target protein of interest and GAPDH, to provide a loading control within the same blot). Visualization of Western blotting results was performed by C-DiGit®Blot Scanner (LI-COR Biotechnology, Lincoln, Nebr.)

f. Assessment of Cell Viability

Cell viability and growth of human MM cell lines were assessed using CellTitre-Glo (CTG) assay (Promega, Madison, Wis.) per kit instructions, or CS-BLI as described (Delmore et al., 2011). Briefly, cells were seeded (5×10³ cells/well) into 96- or 384-well opaque plates, in supplemented media (40 or 50 uL) and incubated for 24 h prior to addition of drug. For co-culture experiments, HS27A cells were pre-seeded (5×10³ cells/well) into each well in media prior (24 h) to the addition of MM cells. At each time point (24 h, 48 h, and/or 72 h) CTG reagent (10% volume) was added to each well, and plates were read using a microplate reader (BioTek Synergy 2, BioTek, Winooski, Va.).

g. Assessment of Apoptosis

For the assessment of apoptosis, MM.1S cells were seeded into 6-well plates (2×10⁶ cells/well) in medium supplemented with 10% FBS and penicillin/streptomycin (3.2 mL) 24 h prior to treatment. Cells were treated with dBET6, JQ1, or vehicle and harvested prior to staining with Annexin V-FITC and PI (BD, Bedford, Mass.). Apoptosis was assessed immediately using flow cytometry (LSR II, BD, Bedford, Mass.)

h. Collection and Ex Vivo Treatment of Patient-Derived Tumor Cells

Samples were processed for separation of CD138+ plasma cells, using CD138-positive selection beads, and immunomagnetic separation, as per kit instructions (EasySep, StemCell, Cambridge, Mass.). Sorted cells were immediately plated into 96-well plates (100 uL media) and cultured overnight or immediately treated with dBET6. Viability was assessed using CTG, as described (24 h, 48 h, 72 h) and each treatment was repeated up to 3 times (n=3 biological replicates).

i. RNA-Sequencing and Reverse Phase Protein Array (RPPA)

MM.1S cells were seeded in 6-well plates (2×106/well) and cultured overnight prior to treatment. After 12 h, dBET6 (100 nM or 500 nM) or JQ1 (500 nM) was added to the cells, then cells were harvested after 4 h or 18 h and immediately frozen (−80° C.). In order to assess transcriptional modulation by dBET6, RNA was extracted as described (Matthews et al., 2015) and, analyzed using next-generation sequencing by the Molecular Biology Core Facilities (MBCF, DFCI). In RPPA analyses, we examined the expression levels of for 303 proteins in a total of 25 RPPA protein expression samples including technical replicates for MM1S cells treated (in 3 separate runs per experimental condition) with dBET6 (100 nM for 4 h and 8 h), JQ1 (500 nM for 4 h and 8 h) and untreated controls. Lysates of these protein samples were processed by the Functional Proteomics RPPA Core Facility of the MD Anderson Cancer Center (Houston, Tex.), based on protocols and procedures outlined in the website of the facility (mdanderson.org/research/research-resources/core-facilities/functional-proteomicsrppa-core/rppa-process.html) and similarly to prior reports (Li et al., 2017). We mapped protein antibody labels to gene symbols available from Antibody Information and Protocols at MDACC (mdanderson.org/content/dam/mdanderson/documents/corefacilities/Functional%20Proteomics %20RPPA%20Core%20Facility/RPPA_Standard%20Ab%20List_Updated.xlsx). For data preprocessing, results from replicates of the same experimental run were averaged for each protein. The resulting matrix of RPPA data was further processed to generate log-transformed linear normalized data, linear normalized, and normalized median centered data. The differential protein expression analysis for dBET6- or JQ1-treated samples vs. controls were performed using the limma moderated t-statistic (Ritchie et al., 2015).

j. In Vivo dBET6 Treatment

i. Subcutaneous MM xenograft model: Luciferase-positive MM.1S cells (10×106 Luc-GFP-MM.1S in matrigel) were transplanted into the right flank of NSG mice (100 μL). Tumor size was measured using calipers. dBET6, prepared daily in Solutol (5%), or vehicle control was injected IP into mice when tumor size reached approximately 100 mm3. Treatments were carried out daily for 8 days and tumor size assessed daily using calipers. Mice were culled when tumors reached 2000 mm3 or when moribund.

ii. Xenograft model of diffuse MM lesions: Luciferase-positive MM.1S cells (1×106 Luc-GFP-MM.1S) were injected via the tail vein (IV) into NSG mice. Mice were imaged (BLI) after 1 week and mice with demonstrable tumor burden were treated with dBET6 or vehicle control daily for approximately 12-14 days. BLI was undertaken weekly and mice were euthanized when moribund or upon signs of hind-limb paralysis.

Animal studies were performed according to a protocol approved by the Dana-Farber Cancer Institute Animal Care and Use Committee.

k. CRISPR/Cas9-Based Gene Editing or Gene Activation Screens to Identify Candidate Mechanisms of Tumor Cell Resistance to Degronimids

MM.1S cells transduced with lentiviral construct for SpCas9 were kindly provided from Quinlan Sievers (Ebert Lab, BWH/DFCI). MM.1S cells were also transduced with lentiviral construct for dCas9-VP64 (purchased from Addgene). We performed genome-scale CRISPR/Cas9 gene-editing screens, similarly to previous studies (Doench et al., 2016; Meyers et al., 2017; Shalem et al., 2014; Wang et al., 2017), and in 3 configurations, which involved: i) “short-term” (48 hours) treatments with either dBET6 or Thal-SNS-032, followed by tumor cell collection at the end of the treatment; ii) “long-term” studies with successive rounds of treatment with dBET6 or Thal-SNS-032 or ARV-771 or MZ-1 for the pools of MM.1S cells transduced with the respective sgRNA library, allowing regrowth between treatments and until in vitro drug sensitivity testing confirmed the selection of pools of MM.1S cells with significant shift-to-the-right of their dose-response curve (compared to degrader-naive controls) for the respective treatment; and iii) “extended degronimid treatment” screens, in which Thal-SNS-032-resistant MM.1S Cas9+ cell populations isolated from our initial “long-term” CRISPR/Cas9-based gene editing screen continue receiving additional degronimid treatment for 2 weeks, with either Thal-SNS-032 (i.e. a continuation of the initial treatment, which led to the isolation of these resistant cells) or dBET6.

These screens were performed with MM.1S-Cas9+ which were transduced with pooled lentiviral particles for genome-scale sgRNA libraries, specifically GECKOv2 for the “long-term” dBET6 screen; and Brunello, for all other genome-wide CRIPSR gene editing studies of degrader-treated cells described in the Examples.

We also performed genome-scale CRISPR-based gene-activation screens, which followed the “long-term” configuration of the gene editing studies. Specifically, MM.1S-dCas9-VP64 cells, which were transduced with pooled lentiviral particles for the Calabrese genome-scale sgRNA library, underwent successive rounds of treatment with dBET6 or ARV-771, allowing regrowth between treatments and until in vitro drug sensitivity testing confirmed the selection of pools of MM.1S cells with significant shift-to-the-right of their dose-response curve (compared to drugnaive controls) for the respective degrader.

i. Production of viral particles: Lenti-X-293T cells (Clontech, Mountain View, Calif.) were plated in T175 culture flasks (0.6×106 cells/mL) in DMEM (Life Technologies) with FBS (10%) for 24 h. After aspiration of cell medium, OPTI-MEM (6 mL) and Lipofectamine 2000 (100 μL; Life Technologies) were added to each flask plus packaging plasmids psPAX2 (20 μg) and MD2. G (10 μg) and plasmid preps of each of the sub-libraries (V2.1, V2.2) of the GeCKOv2 genome-scale sgRNA library or for the Brunello sgRNA library or Calabrese sgRNA library (20 μg per prep; lenti-Cas9-Blast/lentiGuide-Puro). The GeCKOv2 library was kindly provided to us by Offir Shalem and Feng Zhang (Zhang Lab, Broad Institute of MIT and Harvard). Plasmid preps for the Brunello sgRNA library were purchased from Addgene (Doench et al., 2016) and Calabrese sgRNA library were purchased from Addgene. The transfected Lenti-X-293T cells were incubated at 37° C. (20 min), topped up with fresh media (25 mL), and then refreshed again after 16 hours. Viral supernatants were collected after 24 h and stored at −80° C. prior to use.

ii. Lentiviral transductions with sgRNA libraries: For the screen with the GECKOv2 sgRNA library, tumor cell transductions were performed in batches of 8×10⁷ cells per sub-library for each replicate. Cells were incubated for 16 hrs in cell medium containing polybrene (2 μg/mL; Santa Cruz Biotechnology), 10 mM HEPES (pH 7.4) (Sigma-Aldrich) and viral prep (4 mL) diluted to achieve an MOI of 0.4. After the end of the incubation with the viral preps, cells were washed and incubated for an additional day. Transduced cells were cultured at an initial density of 1×10⁶ cells/mL and were treated with puromycin (1 μg/mL) for up to 14 days immediately after transduction. After puromycin selection, MM.1S-Cas9⁺ cells transduced with each GeCKOv2 sublibrary were plated at 60×10⁶ cells per flask (T175, 100 mL) to enable coverage of ˜1000× and were sub-cultured at four to five-day intervals to prevent confluence. At each passage, cells were harvested, washed with PBS (Corning, N.Y.), pelleted (5000 rpm, 5 min, 4° C.) and frozen as cell pellets (−80° C.) for next generation sequencing, and also replated at 60×106 cells to maintain 1000× coverage.

For the genome-scale screens with the Brunello sgRNA library, tumor cell transductions were performed in batches of 5×107 cells per library for six replicates. Cells were incubated (18 h) in cell medium containing polybrene (5 μg/mL; Santa Cruz Biotechnology), 10 mM HEPES (pH 7.4) (Sigma-Aldrich) and viral prep (30 mL) diluted 1:1. Transduced cells were cultured at an initial density of 1×106 cells/mL and were treated with puromycin (1 μg/mL) for up to 5 days additional two days from transduction. After stable transduction, pooled MM.1S cells were plated at 40×10⁶ cells per flask (T175, 100 mL) to enable coverage of 500× and were sub-cultured at three- to four day intervals to prevent confluence. At each passage, cells were harvested, washed with PBS (Corning), pelleted (5000 rpm, 5 min, 4° C.) and frozen as cell pellets (−80° C.) for next generation sequencing, and also replated at 40×10⁶ cells (˜500× coverage).

For the genome-scale screen with the Calabrese sgRNA library, tumor cells were transduced in batches of 3×10⁷ cells per sublibrary for triplicates. Cells were incubated (18 h) in cell medium containing polybrene (4 μg/mL; Santa Cruz Biotechnology), 10 mM HEPES (pH 7.4) (Sigma-Aldrich) and viral prep (30 mL) diluted 1:1. Transduced cells were cultured at an initial density of 1×10⁶ cells/mL and were treated with puromycin (1 μg/mL) for up to 7 days additional two days from transduction. After stable transduction, pooled MM.1S cells were plated at 30×10⁶ cells per flask (T175, 100 mL) to enable coverage of 500× and were sub-cultured at three- to four-day intervals to prevent confluence. At each passage, cells were harvested, washed with PBS (Corning), pelleted (5000 rpm, 5 min, 4° C.) and frozen as cell pellets (−80° C.) for next generation sequencing, and also replated at 30×10⁶ cells per sub-library (˜1000× coverage).

iii. Generation of drug-resistant populations of tumor cells harboring CRISPR editing: As outlined earlier, we generated treatment-resistant tumor cell populations after “long-term” treatment with dBET6, Thal-SNS-032, ARV-771, MZ-1, JQ1 or Bort; “short-term” (48 hours) treatments with either dBET6 or Thal-SNS-032; iii) “extended treatment”, with dBET6 or Thal-SNS-032, for Thal-SNS-032-resistant MM.1 S Cas9+ cell populations isolated from a “long-term” Thal-SNS-032 treatment screen and all treatments were performed triplicates. For “long-term” treatment screens with dBET6, JQ1 and Bortezomib, drug treatments were carried out on MM.1S Cas9⁺ cells transduced with pools of the GECKOv2 sgRNA sub-libraries (≤14 days post-puromycin, 60×10⁶/flask, ˜1000× coverage, 100 mL, n=3 individual flasks per sgRNA sublibrary) 24 h after seeding, as follows: a) for dBET6, cells were treated with drug (0.25 μM) for 24 h prior to complete drug washout. In total, MM.1S Cas9⁺ cells were treated 3 times with dBET6; b) for Bortezomib, cells were treated with drug (0.025 μM) for 24 h prior to drug washout. In total, MM.1S Cas9+ cells were treated 3-4 times with Bortezomib; c) for JQ1, cells were treated with drug for 72 h (0.5 μM) prior to drug washout. In total, MM.1S Cas9+ cells were treated 3-4 times with JQ1; or d) appropriate vehicle controls treated per individual drug protocols. Following treatments, all cells were serially cultured without drug and allowed to regrow for as long as required to enable reseeding (60×106 cells per flask, 100 mL) and retreatment with individual drugs while maintaining ˜1000× coverage throughout. Concomitant with retreatment, remaining cells were frozen (−80° C.; 20% FBS/DMSO) for later analysis or assessed for drug resistance/sensitivity using CTG assays. Between treatments, and in order to maintain the health of cultures, dead cells were periodically removed using Ficoll centrifugation (1,500 rpm for 10-20 min). For all CRISPR drug treatment experiments, aliquots of MM.1S-Cas9+ cells transduced with the GeCKO(Sanjana et al., 2014; Shalem et al., 2014) or Brunello (Doench et al., 2016) sgRNA libraries were frozen immediately prior to experimentation to determine the baseline distribution of sgRNAs in the cell population.

For “long-term” treatment screens with Thal-SNS-032-, ARV-771- and MZ-1-resistant cells, we continuously treated MM.1S-Cas9+ cells (40×10⁶, —500× coverage) transduced with the Brunello sgRNA library (4 weeks post-puromycin selection) with either each compound's (IC20) or vehicle control. Cells were serially passaged every 3-4 days to prevent confluence, each time replating at ˜500× coverage (100 mL) at the same time replenishing each compound or vehicle. Drug treatments were paused if cell numbers fell below 10×106 per flask, allowing cells to recover and regrow, and treatments were resumed at a lower concentration when cell numbers reached 40×10⁶. At each passage, cells were assayed for drug sensitivity/resistance using CTG. After 7 weeks of incubation with Thal-SNS-032 and 6 weeks of incubation with ARV-771 or MZ-1 treatment, cells were collected for next generation sequencing; or Thal-SNS-032 treated-resistant cells were processed for “extended treatment” screens with either: a) an additional treatment with Thal-SNS-032 for 2 weeks; orb) “switch” to dBET6 treatment (50 nM and increased to 100 nM) again for 2 additional weeks.

For “short-term” treatment screens with Thal-SNS-032 or dBET6, MM.1S-Cas9⁺ cells (80×10⁶) transduced with the Brunello sgRNA library were treated with either Thal-SNS-032 (25 nM) or dBET6 (25 nM) or vehicle control for 48 hrs. Cells were immediately harvested (30×10⁶) and sgRNA distribution assessed, by next generation sequencing. For genome-scale CRISPR activation screens of dBET6 or ARV-771 resistant cells, we continuously treated MM.1S-dCas9-VP64 cells (30×10⁶, —500× coverage) transduced with the Calabrese sgRNA library (2 weeks post-puromycin selection) with either each compound's (IC20) or vehicle control as described above. After 5 weeks of incubation with of incubation with dBET6 or ARV-771 treatment, cells were collected for next generation sequencing

iv. Next generation sequencing: Preparation of DNA for next generation sequencing was undertaken using a two-step PCR protocol as described (Shalem et al., 2014). Briefly, DNA was extracted from frozen cell pellets (2 or 3×10⁷ cells; Blood & Cell Culture DNA Midi Kit or Maxi Kit, Qiagen) per manufacturer's instructions. DNA concentration was quantified by UV-spectroscopy (NanoDrop 8000; ThermoFisher Scientific). In the first PCR, sgRNA loci were selectively amplified from a total of 130 μg or 160 μg of genomic DNA (10 μg DNA per sample×13 reactions, 100 μL volume for GeCKO and Calabrese library or 16 reactions for Brunello library) using primers described in Table S1 and Phusion® High-Fidelity DNA Polymerase (New England Biolabs, Beverly, Mass.). This provides approximately 300× coverage for sequencing. A second PCR was performed using 5 μL of the pooled Step 1 PCR product per reaction (1 reaction per 10,000 sgRNA's; 100 μL reaction volume) to attach Illumina adaptors and to barcode samples (Table S1). Primers for the second PCR included a staggered forward primer (to increase sequencing complexity) and an 8 bp barcode on the reverse primer for multiplexing of disparate biological samples (Table S1). PCR replicates were combined, gel normalized (2% w/v) and pooled, then the entire sample run on a gel for size extraction. The bands containing the amplified and barcoded sgRNA sequences (approximately 350-370 bp) were excised and DNA extracted (QIAquick Gel Extraction Kit, Qiagen). Multiplexed samples were then sequenced at the Molecular Biology Core Facility (Dana-Farber Cancer Institute) and/or The Genomics Platform (Broad Institute) using an Illumina NextSeq 500 (Illumina, San Diego, Calif.), allowing 4×108 individual reads per multiplexed sample.

l. CRISPR/Cas9-Based Gene Knockouts with Individual sgRNAs to Validate Candidate Resistance Genes.

We performed genome-scale CRISPR/Cas9 gene-editing screens, similarly to previous studies (Doench et al., 2016; Meyers et al., 2017; Shalem et al., 2014; Wang et al., 2017), and in 3 configurations, which involved: i) “short-term” (48 hours) treatments with either dBET6 or Thal-SNS-032, followed by tumor cell collection at the end of the treatment; ii) “longterm” studies with successive rounds of treatment with dBET6 or Thal-SNS-032 or ARV-771 or MZ-1 for the pools of MM.1S cells transduced with Individual sgRNAs against candidate degronimid resistance genes (e.g. COPS2, COPS3, COPS7A, COP S8) were designed using the Broad Institute sgRNA design portal (https://portals.broadinstitute.org/gpp/public/analysistools/sgrna-design). Non-targeting control sgRNAs and COPS7B sgRNAs were from GeCKOv2. Four sgRNAs were generated per each gene, except from COPS7B, for which 6 sgRNAs were available (Table S2). All sgRNAs were synthesized by CustomArray Inc (Bothell, Wash.). Cloning was performed according to the protocol published by Zhang et al. (media.addgene.org/cms/filer_public/4f/ab/4fabc269-56e2-4ba5-92bd-09dc89c1e862/zhang_lenticrisprv2_and_lentiguide_oligo_cloning_protocol_1.pdf) using a pHKO9 vector. Briefly, to clone the sgRNA guide sequence, plasmids were digested with BsmBI (New England Biolabs, Ipswich, Mass.) at 55° C. for 1 hour. Oligonucleotides for each sgRNA guide sequence were annealed and phosphorylated using T4 Ligation Buffer and T4 polynucleotide kinase (New England Biolabs) at 37° C. for 30 minutes and then annealed by heating to 95° C. for 5 minutes and cooling to 25° C. at 1.5° C./minute. Using T4 ligation buffer and T4 ligase (New England Biolabs), annealed oligos were ligated into gel purified vectors (Qiagen) at 65° C. for 10 minutes. 2.5 ug of the ligation product were transformed in 30 uL E. coli electrocompetent cells (Lucigen E.cloni 10 G ELITE PLUS, Invitrogen). Subsequently, 500 uL of transformed cells were plated on LB Agar-AmpR (ThermoFisher Scientific) and incubated overnight at 37° C. Three colonies per plate were then picked and inoculated in a mini-prep culture (Qiagen). The product was then digested with BsmBI and XhoI (New England Biolabs) at 37° C. for 1 h and compared to the uncut control on 1% agarose gel. One out of three colonies (10 uL) was then precultured in 3 mL LB Broth-AmpR and shaken at 37° C. for 6 h. Afterwards it was transferred to a 250 mL LB Broth-AmpR flask and incubated overnight at 37° C. Each culture was then inoculated into a maxiprep culture (Qiagen).

Also, CRBN, VHL, TCEBJ, TCEB2, UBE2R2 and FBXW2 sgRNA are selected from Brunello library and cloned using pLVX-hyg-sgRNA1 Vector system (Takara Bio USA, CA) according to the manufacturer's manual. b(takarabio.com/assets/documents/User %20Manual/Lenti-X_CRISPR-Cas9_System_User_Manual_121316.pdf)

i. Production of viral particles: Lenti-X-293T cells (Clontech, Mountain View, Calif.) were plated in 6-well plates (1.5×10⁶ cells/well) in DMEM (Life Technologies) with FBS (10%) for 24 h. After aspiration of cell medium, OPTI-MEM (6 mL) and Lipofectamine 2000 (100 μL; Life Technologies) were added to each flask plus packaging plasmids psPAX2 (20 μg) and MD2. G (10 μg) and plasmid preps of each of the constructs (1300 ng per prep). The transfected Lenti-X-293T cells were incubated at 37° C. (20 min), topped up with fresh media (2 mL per well), and then refreshed again after 16 hours. Viral supernatants were collected after 24 h and stored at −80° C. prior to use.

ii. Lentiviral transductions with constructs for individual sgRNAs: 500×10³ to 1×10⁶ MM.1S Cas9 cells were plated in 50 uL of complete RPMI1640 medium per well in a 24-well plate, and additional 100 uL of complete RPMI1640 medium were added to cover the well completely. Cells were incubated in cell medium containing polybrene (5 to 8 μg/mL; Santa Cruz Biotechnology), and viral prep. 24 hours after addition of viral preps, media were changed and, after another 48 h, puromycin selection (1 to 2 ug/ml per well) was started. After 7 days of puromycin selection, transduced MM.1S Cas9+ cells were collected and expanded in T75 flasks.

iii. Dose-response assay: Knock-out cells and non-targeting controls were subsequently plated in 96-well or 384-well plates as described and treated with dBET6 at 0, 0.01, 0.05, 0.1, 0.5 and 1 uM; and viability was assessed after 24, 48 and 72 h using CTG. In the case of Thal-SNS-032 or ARV771, cells are plated 384-well plate and treated each concentration described at figure and viability assays were performed at 72 hrs.

m. Depletion of CRBN in Human MM Cell Lines

Human MM cell lines KMS11 and OPM-2 expressing non-targeting control shRNAs or shRNA. CRBN #13 obtained from Keith Stewart and cultured (Kolde et al., 2012), as previously described by Zhu et al. (Zhu et al., 2013), were treated with dBET6, and cell viability was measured using CTG, as described in prior sections.

n. Quantification and Statistical Analysis of CRISPR Data

We performed 3 Illumina HISEQ with 4 lanes each for the dBET6 experiment (GeckoV2) and 1 Illumina HISEQ with 4 lanes for the ZZ1 experiment (Brunello). In order to determine the technical experimental variability of the assay, we performed sample preparations of multiple in-culture time points (2 weeks, 6 weeks and 12 weeks). The reads of a HISEQ were run in 4 lanes and were demultiplexed into the individual barcoded samples and corresponding technical replicates from 4 lanes were merged. Demultiplexed sequencing reads for each sample were mapped to the sgRNA library using bowtie. The GeckoV2 (addgene.org/crisprlibraries/geckov2/) sub-libraries A and B were merged and duplicated sgRNA sequences were removed for the read quantification mapping using bowtie. For the sequencing, we designed staggered primers to increase the read complexity for the illumina sequencing procedure. We removed staggered primer adapters from the raw reads (BROAD Walk-up sequencing facility) and 5′ adapters using cutadapt (v.1.9.1, Marcel Martin, journal.embnet.org/index.php/embnetjournal/article/view/200). The trimmed reads (20mers) are aligned to the respective sgRNA library using bowtie2 (using the parameter settings --norc --local -D 20 -R 3 -N 0 -L 10 -i S,1,0.5 -p 6 for a highly sensitive alignment search). For each sample we filtered the reads for mapped read alignments for unique matching reads with at most 1 base mismatch and estimated the respective count frequencies for each sgRNA. The feature count matrix and sgRNA matrix formatting was performed in the script language R. The paired samples preparation of the GeckoV2 sub-libraries A and B were joined and the technical replicates of the dBET6 experiment were merged by the summation of read counts of the corresponding sgRNAs. We performed a one-sided test for enrichment and depletion of the sgRNAs and sgRNA rank aggregation for each gene using the Mageck-RRA algorithm using default parameter settings (Li et al., 2014). We performed the analysis for three readcount normalization techniques (total, median and non-targeting read count normalization). The non-targeting sgRNAs was used as control distribution for the rank aggregation procedure based on the RRA algorithm (Kolde et al., 2012)

### Example 2: Pre-Clinical Anti-Myeloma Activity and Molecular Sequelae of Pharmacological BET Bromodomain Degradation

In this study, genome-scale loss-of-function (LOF) screens were performed using the Clustered Regularly Interspaced Short Palindromic Repeats (CRISPR) technology, to identify the mechanisms of resistance of multiple myeloma (MM) cells to degronimids against the BET bromodomain proteins BRD4, 3 and 2 (dBET6) (Winter et al., 2017) and against CDK9 (Thal-SNS-032) (Olson et al., 2018); as well as degraders of BRD4, 3 and 2 which operate through a different E3 ligase, VHL, namely ARV-771 (Raina et al., 2016; Saenz et al., 2017; Sun et al., 2017) and MZ-1 (Gadd et al., 2017; Zengerle et al., 2015). The goal of these studies was to identify, in an unbiased manner, genes for which the LOF confers to cancer cells, such as MM cells, resistance to degronimids; whether any of these genes are involved in clinical resistance to the prototypical degronimids, thalidomide and its analogs; and which mechanisms of resistance are common vs. distinct for CRBN- vs VHL-mediated pharmacological degraders.

The BET Bromodomain protein BRD4 regulates the expression and function of the c-Myc oncoprotein (Delmore et al., 2011); and the BET Bromodomain inhibitor (BBI) JQ1 has anti-tumor activity in preclinical in vitro and in vivo models of MM (Delmore et al., 2011) and other neoplasms (e.g. (Loven et al., 2013; Zuber et al., 2011)). However, treatment with BBIs causes compensatory upregulation of BRD4 protein levels (Lu et al., 2015; Winter et al., 2015): this event could in principle attenuate the antitumor activity of BBIs, and explain why JQ1 does not induce apoptosis in most cell types tested (Delmore et al., 2011; Zuber et al., 2011) and does not achieve cure, even after complete biochemical remissions (e.g. in the Myc-driven Vk*MYC MM model (Delmore et al., 2011)). We thus examined if BET bromodomain-targeting degronimids achieve more potent anti-MM activity. Consistent with prior reports on the original BET bromodomain-targeting degronimid dBET1 (Winter et al., 2015; Winter et al., 2017) and its lead-optimized successor, dBET6 (Winter et al., 2017), we observed more potent in vitro anti-MM activity of dBET6 compared to dBET1 or JQ1 (FIG. S1) and thus focused on dBET6, as a representative degronimid against BET bromodomain proteins, for the remainder of our study. We confirmed that dBET6 causes rapid and robust depletion of BRD4, BRD3, BRD2 and c-Myc, while JQ1 causes less pronounced depletion of c-Myc and compensatory increase in BRD4 protein levels (FIGS. S2a-c). In contrast to prior studies with JQ1 (Delmore et al., 2011), dBET6 induces in human MM.1S cells a dose- and time-dependent pro-apoptotic response (FIG. S3a): and dBET6 exposure for as little as 4 hrs prior to drug washout is sufficient to commit MM.1S cells to cell death (detected by flow cytometry for Annexin V/PI staining) (FIG. S3b). No major changes in dBET6 responsiveness were noted when MM cells were cultured in the presence of bone-marrow stromal cells (FIG. S3c), which are known to induce cell resistance to diverse established or investigational agents in MM and other bone-homing neoplasias (McMillin et al., 2012; McMillin et al., 2010). When we compared the transcriptional (RNA-Seq) and proteomic (reverse phase protein arrays; RPPA) profiles of dBET6 vs JQ1-treated MM.1S cells, we observed that dBET6 induced down-regulation of a larger group of transcripts (FIG. S4a); more pronounced suppression of c-MYC at both transcript and protein level (FIGS. S2d, S4b). Importantly, dBET6 treatment at 4 hrs, but not JQ1, causes downregulation of Mcl-1, Cyclin B1 (not shown), and BRD4; followed at later time-points with suppression of Ser240/Ser244 S6 phosphorylation; induction of p21, and increase in cleaved caspase3/7 (FIG. S4c). The concomitant suppression of anti-apoptotic molecules (e.g. Mcl-1) and induction of pro-apoptotic events (e.g. caspase cleavage) by dBET6, but not JQ1, provides mechanistic explanation for the robust induction of cell death/apoptosis by the former and not the latter. We observed significantly lower tumor burden in dBET6- vs. vehicle-treated mice in two models of subcutaneous and disseminated, respectively, growth of human MM.1S cells in immunocompromised mice (FIGS. S5a,c); with prolongation of survival in dBET6-treated mice with subcutaneous MM xenografts (FIG. S5b). Mice tolerated dBET6 treatment at the doses and schedules reported, but in other pilot experiments (data not shown), alternative regimens with lower or higher dose intensity had less efficacy or tolerability, respectively; suggesting that development of novel preparations of dBET compounds with optimized delivery to the tumor sites is required.

### Example 3: Anti-Tumor Activity of dBET6 in the Context of Decreased Responsiveness to Other Therapeutics

We examined whether dBET6 is active against 2 distinct groups of MM cells with decreased responsiveness to other established or investigational therapeutics, namely a) pools of MM.1S with decreased responsiveness to bortezomib or JQ1 (FIG. S6a,b) after being exposed to a pooled whole-genome library of sgRNAs for CRISPR/Cas9-based gene editing and then receiving treatment with bortezomib or JQ1; and b) samples of patient-derived MM cells from clinical cases with variable patterns of exposure to and resistance/refractoriness to currently available clinical treatments (FIG. S6d). For both sets of studies, we observed that prior resistance to other therapies does not preclude responsiveness to dBET6. For example, the pools of MM.1S cells with gene editing-mediated tolerance to bortezomib (FIG. S6a) or JQ1 (FIG. S6b) exhibited dBET6 sensitivity which was equal to or greater than treatment-naive cells transduced with the same genome-scale library of sgRNAs (FIG. S6c). Consistent with the human MM cell line data, patient-derived cells exhibited dose-dependent response to dBET6, with individual patient samples demonstrating a broad range of sensitivities (FIG. S6d), without any obvious clustering of the dose-response patterns according to the status of prior therapies (e.g. relapsed/refractory vs. on maintenance therapy vs. newly diagnosed vs. MGUS).

### Example 4: CRISPR-Based Functional Genomic Characterization of Mechanisms of MM Resistance to CRBN Mediated Degraders

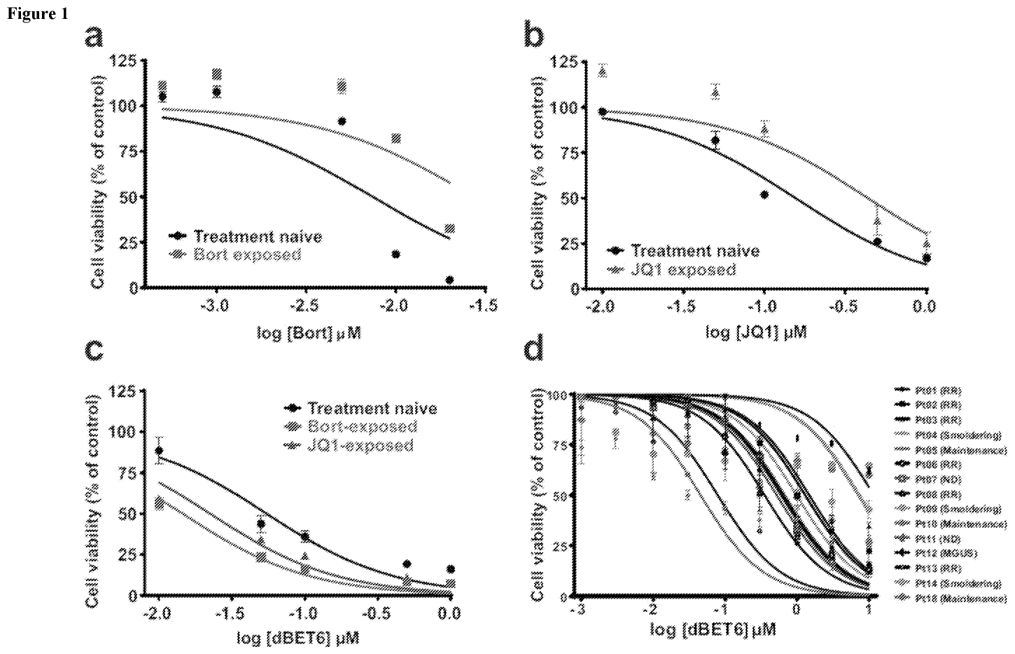

We performed genome-scale CRISPR/Cas9 gene editing screens (FIG. S7) in dBET6-treated cells to perform unbiased identification of candidate genes that can confer potential resistance to this compound. MM.1S-Cas9+ cells were transduced with pooled lentiviral particles of the GeCKOv2 library and were either treated with dBET6 (0.25 μM) or control. After three rounds of treatment with dBET6, we observed the outgrowth of dBET6-resistant cells (FIG. 1a) and performed PCR amplification and next-generation sequencing (NGS) of the sgRNA barcodes to quantify which genes were subject to sgRNA enrichment or depletion in these cells. We observed significant enrichment of sgRNAs targeting CRBN itself, as well as other components or regulators of the cullin-RING ligase (CRL) complex, including members of the human COP9 signalosome complex (COPS7A, COPS7B, COPS2, COPS3, COPS8, GPS1, etc.), DDB1, or the E2 ubiquitin conjugating enzyme UBE2G1 (FIG. 1b). Transduction with individual sgRNAs for COPS7B (FIG. 1c), COPS8 (FIG. 1d) or CRBN (FIG. 1e-f) decreases the response of MM.1S cells to dBET6.

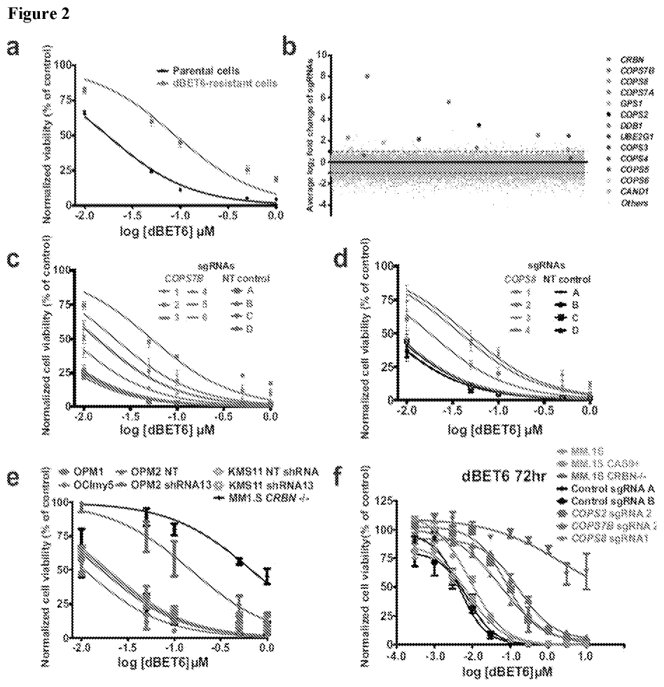

We performed a similar CRISPR-based genome-scale screen (FIGS. S7 and 2a) for resistance to another CRBN-based degronimid, Thal-SNS-032, which causes degradation of CDK9 (FIG. S2). Again, there was pronounced enrichment of sgRNAs for CRBN itself, and COP9 signalosome complex genes (COPS7A, COPS7B, COPS2, COPS3, COPS8, GPS1, etc.), DDB1, or UBE2G1 (FIG. 2b). These results were concordant with our dBET6 studies; and a distinct CRISPR gene editing study (Sievers et al., 2018) to identify mechanisms of MM.1S cell resistance to lenalidomide. We again validated that individual sgRNAs for several of these candidate genes (e.g. COPS7B, COPS2, DDB1, or COPS8; FIG. 2c-f, respectively) can decrease the Thal-SNS-032 responsiveness of MM.1S cells.

Decreased CRBN transcript levels or alternative splicing (Gandhi et al., 2014; Heintel et al., 2013), but typically not biallelic LOF (biallelic deletion or LOF mutations), are detected in tumor cells of MM patients with clinical resistance to thalidomide and its derivatives (Heintel et al., 2013; Zhu et al., 2011), i.e., the prototypical degronimids. We studied MM cell lines previously reported (Heintel et al., 2013; Zhu et al., 2011) to have decreased response to lenalidomide due to low, but detectable, levels of CRBN, either constitutively (OPM1 and OCI-MY5) or after transduction with shRNAs against CRBN (KMS11, OPM2). We observed that these lines (FIG. 2e) remained responsive to dBET6. These results collectively suggest that low CRBN levels which may not be sufficient for the usual natural therapeutic effects of IMIDs, such as degradation of IZKF1, may still allow CRBN-mediated degraders to maintain, at least in part of the cases, substantial levels of activity, offering potential therapeutic opportunities for resistant patients.

### Example 5: Kinetic Analyses of CRISPR Screens for Resistance to Degronimids Reveal Dynamics of LOF for CRBN Vs. Non-CRBN Resistance Genes

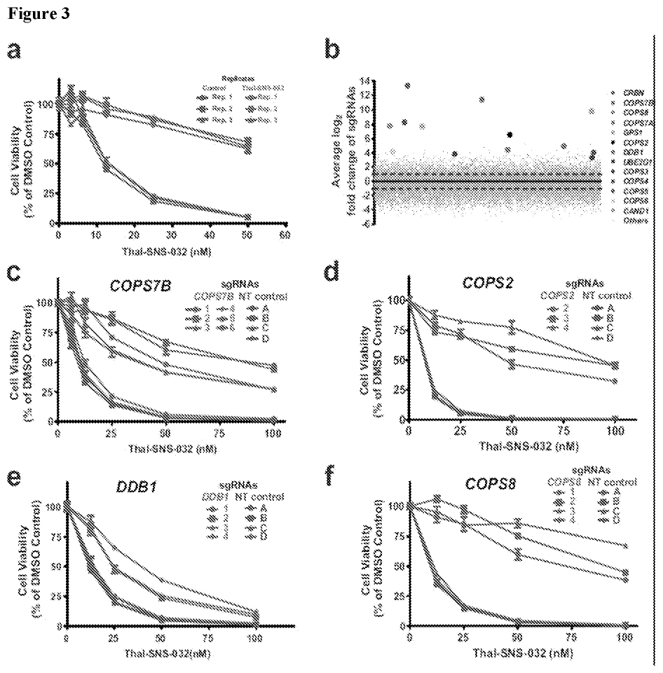

We examined the dynamics of MM cell populations with LOF for degronimid resistance genes at different time points of our genome-scale CRISPR screens and specifically hypothesized that sgRNAs for CRBN should demonstrate over time progressively more pronounced enrichment compared to sgRNAs for other candidate degronimid resistance genes identified from our studies. To address this hypothesis, we performed, for each degronimid of our study, two distinct types of genome-scale CRISPR/Cas9-based gene editing screens (FIG. S7), namely i) “short-term” screens, in which MM.1S Cas9+ cells transduced with the genome-scale sgRNA library Brunello were exposed to shortterm (48 hour) treatments with either dBET6 or Thal-SNS-032 (FIGS. 3a,b) and then tumor cells were collected at the end of the treatment for PCR-NGS; and ii) “extended degronimid treatment screens, in which Thal-SNS-032-resistant MM.1S Cas9+ cell populations isolated from our initial “longterm” CRISPR/Cas9-based gene editing screen continue receiving additional degronimid treatment for 2 weeks, with either Thal-SNS-032 (i.e., a continuation of the treatment, which led to the isolation of these treatment-resistant cells) or dBET6 (FIG. 3c). We observed that Thal-SNS-032-resistant MM.1S cells established from our CRISPR screen, upon re-challenge with either Thal-SNS-032 or dBET6 for an additional 2 weeks of in vitro culture led to further enrichment of MM cells containing sgRNA against CRBN, but not against other “hits” identified from our earlier “long-term” screens with either degronimid tested.

### Example 6: CRISPR Studies to Identify Resistance Mechanisms to VHL-Based Degraders Targeting BRD2/3/4

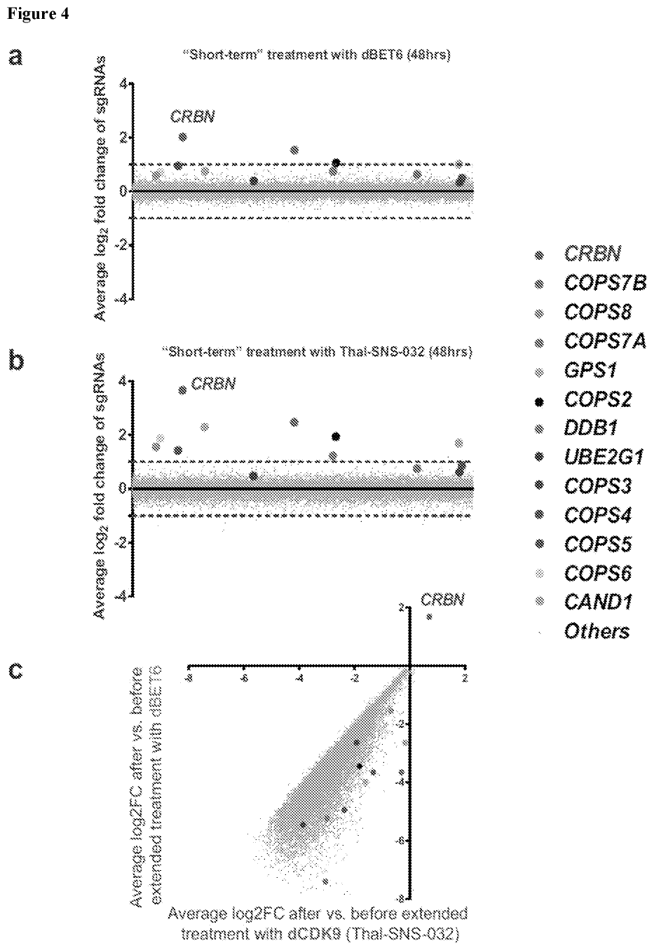

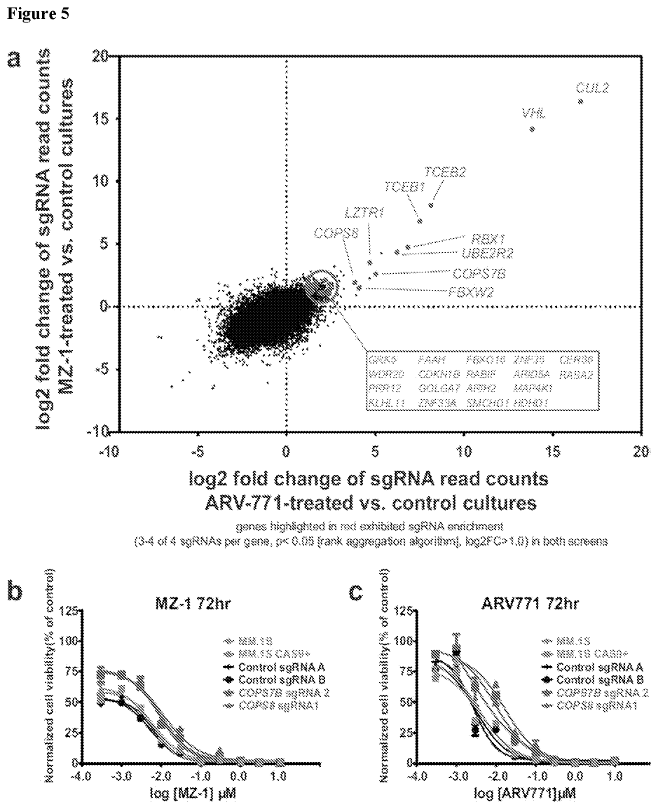

Given that resistance to dBET6 or Thal-SNS-032 is mediated primarily through LOF of CRBN and its regulators, we hypothesized that tumor cell resistance to degraders operating through a different E3 ligase and cullin-RING ligase complex would lead to a different set of candidate resistance genes related to the function of these latter molecules. We thus performed additional CRISPR-based genomescale screens for resistance against heterobifunctional agents (ARV-771 (Raina et al., 2016; Saenz et al., 2017; Sun et al., 2017) and MZ-1 (Gadd et al., 2017; Zengerle et al., 2015)) which cause BET protein degradation via the E3 ubiquitin ligase activity of VHL. In this case, we observed (FIG. 4a) enrichment of sgRNAs for CUL2, VHL itself, other members (e.g. RBXJ, elongin BC [TCEBJ, TCEB2] (Kibel et al., 1995; Lonergan et al., 1998) of the CUL2 complex with VHL), as well as COPS signalosome complex genes (COPS7B, COPS8) or UBE2R2. When we treated MM.1S cells harboring sgRNAs against COPS7B or COPS8 with the MZ-1 or ARV-771, we observed decreased response (compared to parental MM.1S cells) (FIGS. 4b,c), although the shift in the dose-response curves for these two VHL-mediated degraders was less pronounced compared to the one observed in experiments with dBET6 (FIG. 1f) or Thal-SNS-032 (FIGS. 2d,f). We also extended our validation studies to additional genes and documented decreased activity of VHL- (but not CRBN-) based degraders against cells with sgRNAs against VHL (MM.1S and KMS-11 cells, FIG. S7a-b), TCEBJ, TCEB2, CUL2, FBXW2, UBE2R2 (MM.1S cells, FIG. S7c,d). In contrast, no significant changes in ARV771 activity was observed against cells with sgRNAs against CRBN (FIG. S7a); or several olfactory receptor genes (FIG. S7a-d) which are not expressed in MM cells and serve as additional negative controls.

### Example 7: Sequential Versus Concomitant Exposure to Degraders Targeting the Same Oncoprotein Through Different E3 Ligases

Our CRISPR studies at genome-scale and the individual gene-level indicate that the genomic determinants of response to different pharmacological degraders targeting the same oncoprotein through different E3 ligases reflect common pathways, but involve different individual genes. This raised the possibility that sequential administration of degraders targeting the same oncoprotein through different E3 ligases could exhibit delay or prevent the development of resistance compared to sequential administration of degraders which target different oncoprotein but engage the same E3 ligase. We addressed this question using pools of MM cells derived from several of our genome-scale CRISPR-based screens. To specifically study the impact of sequential treatment with CRBN-based degrader followed by VHL-based degrader, we examined pools of MM cells which had survived CRISPR-based studies after (i) “long-term” treatment with Thal-SNS-032; “long-term” treatment with Thal-SNS-032 followed by (ii) “extended” treatment with Thal-SNS-032 or (iii) extended treatment with dBET6; vs. populations of drug-naïve cells which remained in culture during the “long-term” or “extended” treatments with these CRBN-based degraders and were collected at the end of the respective studies. We then exposed each of these populations (3 replicate pools for each population) to dBET6, Thal-SNS-032, ARV-771 or MZ-1: we observed that pools of MM cells previously exposed to the CRBN-based degraders (dBET6, Thal-SNS-032) exhibited, compared to treatment-naïve cells, major shifts to the right for their dose response curves against these same CRBN-based degraders, but limited, if any, shift for their response to the VHL-based degraders ARV-771 or MZ-1 (FIG. S8a). We also performed similar experiments to address the impact of the reverse sequence of treatment, namely initial exposure to VHL-based degraders, followed by treatment with a CRBN-based degrader: We observed that pools of MM cells previously exposed to ARV771 and then MZ1 had similar responses to dBET6 as treatment-naïve cells, but had significantly decreased responsiveness to ARV771 (FIG. S8b).

The results from sequential administration of degraders leveraging different E3 ligases raised the question whether concomitant administration of such degraders would also lead to enhanced antitumor activity. We thus examined the simultaneous exposure of MM.1S cells to combinations of Thal-SNS-032 plus dBET6 (FIG. S9a,b); Thal-SNS-032 plus ARV-771 (FIG. S9c,d); and dBET6 plus ARV-771 (FIG. S9e,f). For the first two of these combinations, increasing concentrations of Thal-SNS-032 were associated with decrease in the % viability of dBET6- or ARV-771-treated cells when compared to either drug-free controls (FIG. S9a,c); or the respective dBET6- or ARV-771-free cultures for each Thal-SNS-032 dose level (FIG. S9b,d). In contrast, for the third combination, increasing concentrations of dBET6 were associated with decreased % viability of ARV-771-treated cells compared to drug-free control cells (FIG. S9e), but lower compared to the respective ARV-771-free cultures for each dBET6 dose level (FIG. S9f). These results indicate that concomitant exposure to two degraders which target the same oncoprotein through different E3 ligases may not necessarily lead to enhanced antitumor activity and may actually lead to antagonistic effects. A possible interpretation of these results is they represent a modified version of the so-called “hook” effect (Bondeson et al., 2015; Buckley et al., 2015; Burslem et al., 2017; Lu et al., 2015; Ohoka et al., 2018; Olson et al., 2018; Schiedel et al., 2017; Winter et al., 2015): high concentrations of an individual degrader have been reported to lead to high concentrations of the individual binary complexes between degrader-target protein and degrader-E3 ligase, thus inhibiting the formation of ternary complexes between target protein-degrader-E3 ligase which are required for target ubiquitination and eventual degradation. In the current setting of simultaneous exposure to two heterobifunctional degraders which target the same protein through different E3 ligases, a similar “hook” effect presumably applies and is perhaps exacerbated by the existence of 2 different ternary complexes (one for each degrader-E3 ligase pair) and four possible binary complexes which compete against each other and prevent target protein ubiquitination.

### Example 8: Functional Genomics Landscape for Degrader-Resistance Genes in Human Tumor Cell Lines

Our CRISPR/Cas9-based gene-editing screens address potential loss of function mechanisms associated with resistance to heterobifunctional degraders. We also performed CRISPR/Cas9-based activation screens in which the Calabrese genome-scale library of sgRNAs against promoters regions of different genes was transduced into MM.1S cells expressing a Cas9 variant which lacks nuclease activity [dCas9] and confers P65-HSF-mediated activation of genes recognized by sgRNAs against their promoter regions. Tumor cells were then exposed to serial treatments with either dBET6 or ARV771, in a manner reminiscent of our “long-term” CRISPR/Cas9 gene-editing loss-of-function studies, i.e. with successive rounds of dBET6 or ARV-771 treatment of the pools of MIM.1S cells with genome-scale CRISPR-based gene activation, allowing re-growth between treatments and until in vitro drug sensitivity testing confirmed the selection of pools of MM.1S cells with significant shift-to-the-right of their dose-response curve (compared to degrader-naive controls) for the respective treatment. From these studies, ABCB1, the gene for the MDR1 transporter, was commonly identified as the only gene with sgRNA enrichment in CRISPR/Cas9 activation studies for both dBET6 or ARV-771 (FIG. S10a,b), consistent with the fact that these large hydrophobic molecules are MDR1 substrates. In the absence of other plausible degrader resistance “hits” identified from these gain-of-function studies, we focused our attention on defining for the degrade resistance hits identified from our loss-of-function studies, their functional genomics landscape in human tumor cell lines.

Because the CRISPR screens of our groups identified CRBN and several other highly concordant “hits” associated with resistance to degronimids against multiple targets (BET bromodomains, CDK9, IKZF1/3), we examined if these genes are associated with potential mechanisms of resistance to thalidomide derivatives in the clinically annotated molecular profiling data of MM patients from the MMRF CoMMpass study (Foulk et al., 2018; Kowalski et al., 2016; Miller et al., 2017). Unlike the association of CRBN expression with clinical outcome, other “hits” from our screens were neither down regulated nor mutated in baseline (pre-treatment) samples from MM patients with inferior clinical outcome (e.g. shorter progression free survival or overall survival) after receiving IMID-containing regimens (whether they contained or not proteasome inhibitors) or in samples collected from MM patients who relapsed after initial response to such IMID-containing regimens (data not shown).

We reasoned that, compared to other hits from our CRISPR screens, LOF for CRBN exhibits a distinct and critical role in clinical resistance to IMIDs, because it does not confer major adverse impact on the proliferation and survival of MM cells. Indeed, as part of our efforts to map out the landscape of molecular dependencies of MM cells through genome-scale CRISPR essentiality screens (in the absence of drug treatments), we noted that most non-CRBN “hits” from our degronimid resistance screens, but not CRBN itself, have significant depletion of their sgRNAs in the MM cell lines tested, as well as in other neoplasms (FIG. 6). These results suggest that LOF of CRBN may provide MM cells with an advantage in the context of degronimid/IMID treatment, because CRBN plays a central role in the mechanism of action of these agents and its LOF, unlike most other candidate degronimid resistance genes, does not confer to tumor cells a “fitness cost” in the form of attenuated proliferation or survival in the absence of treatment.

### Example 9: Functional Genomics Landscape for E3 Ligases in Human Tumor Cell Lines

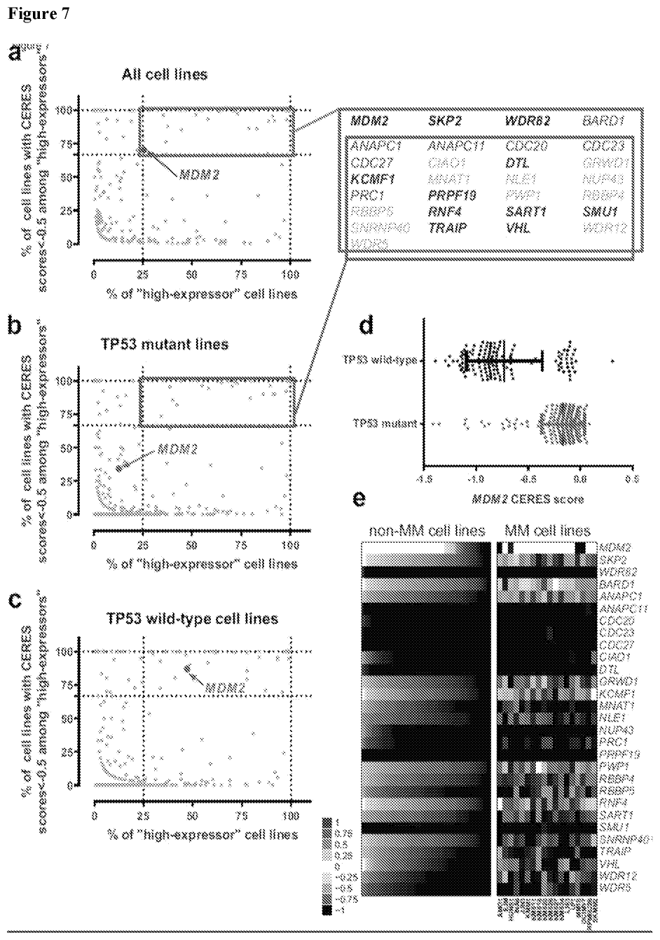

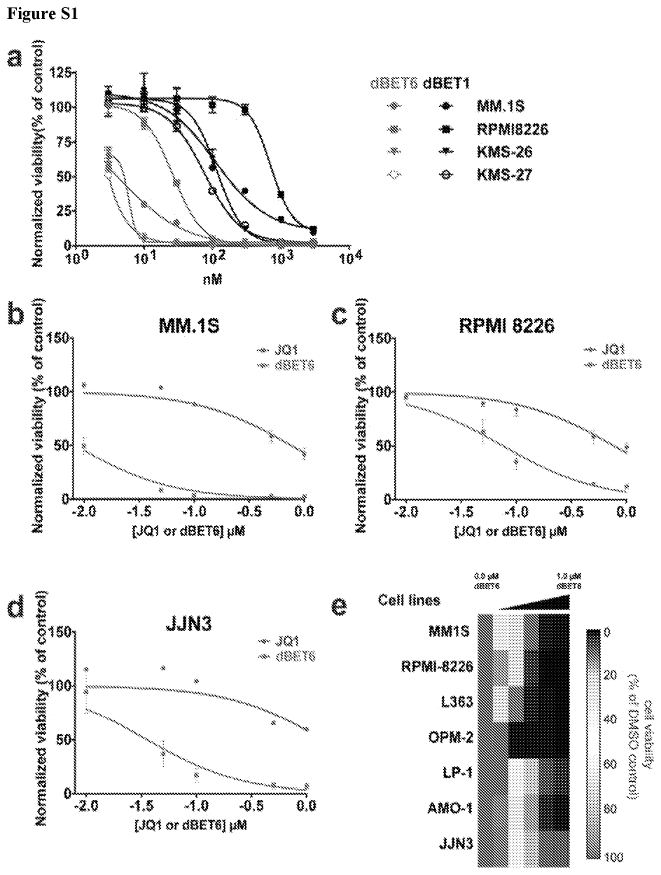

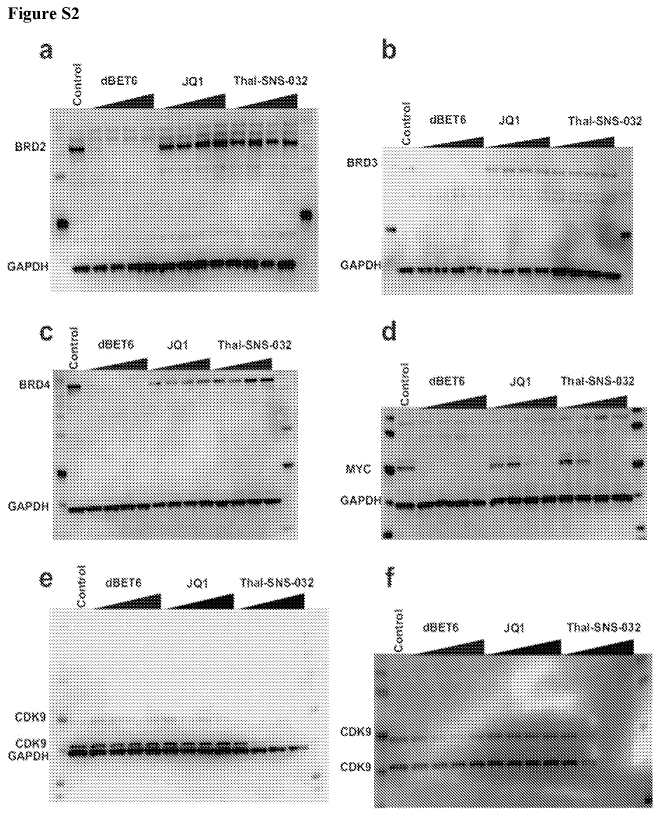

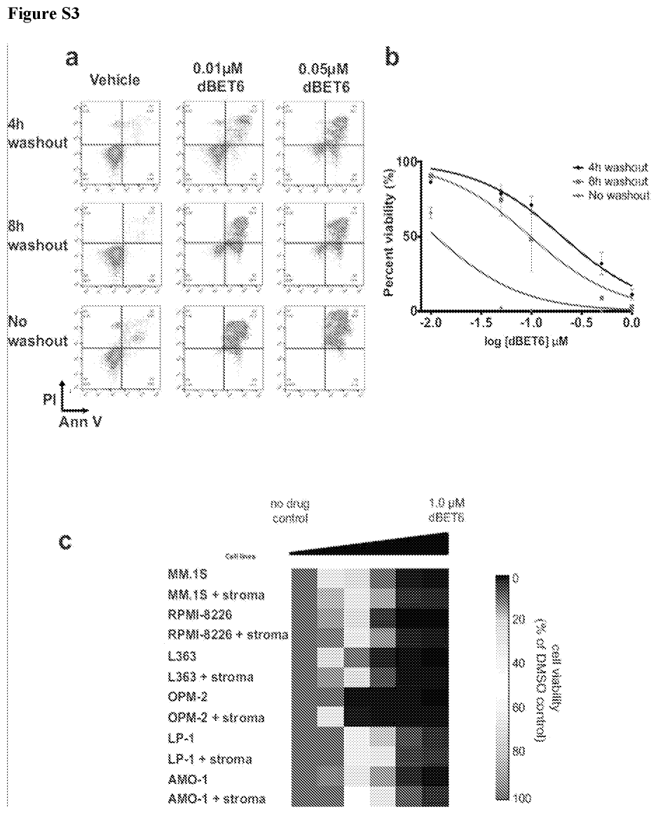

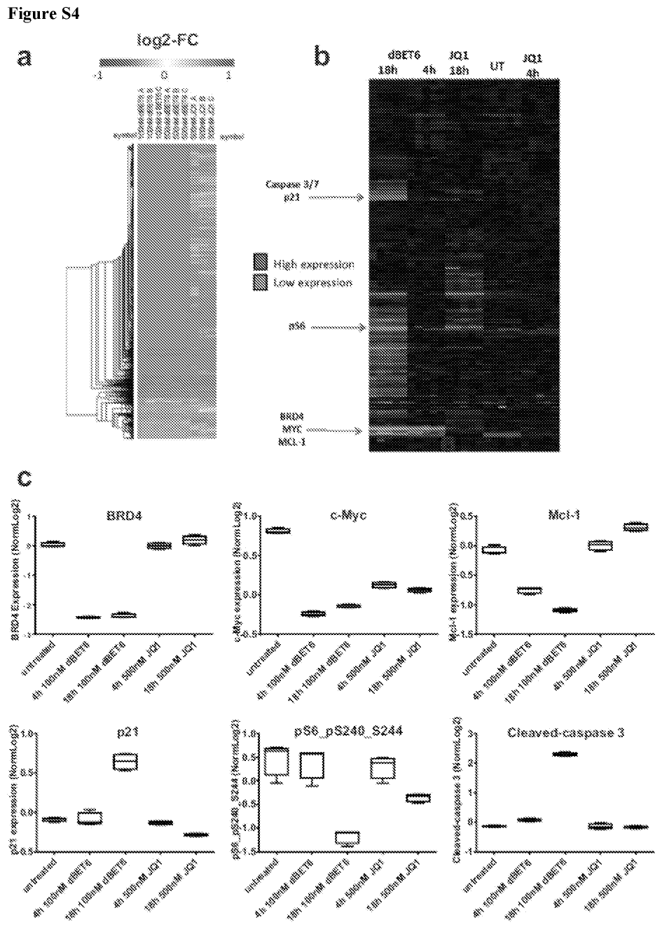

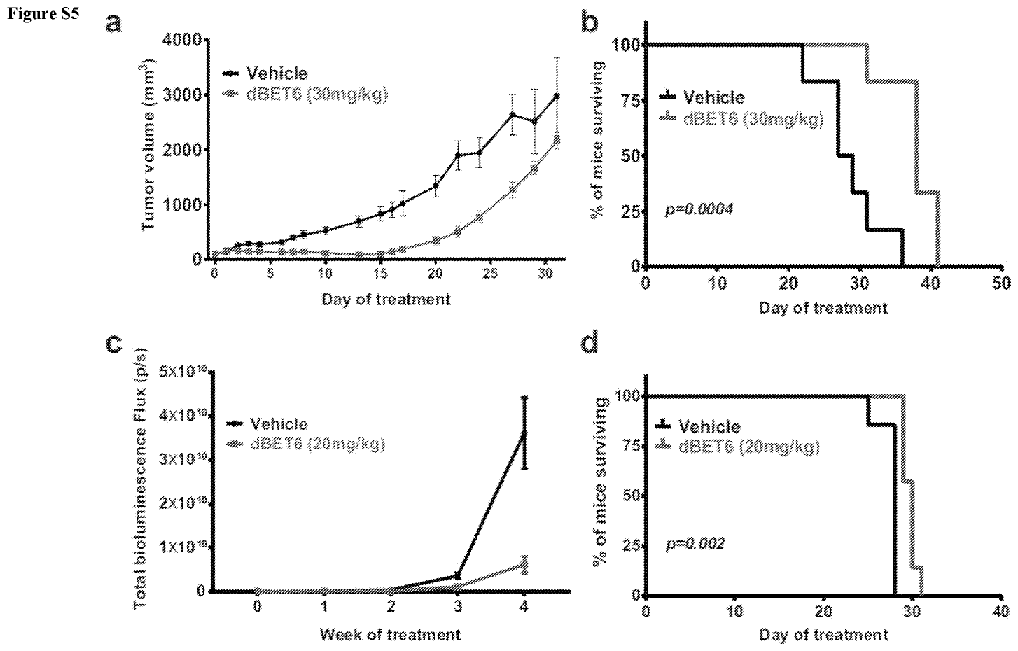

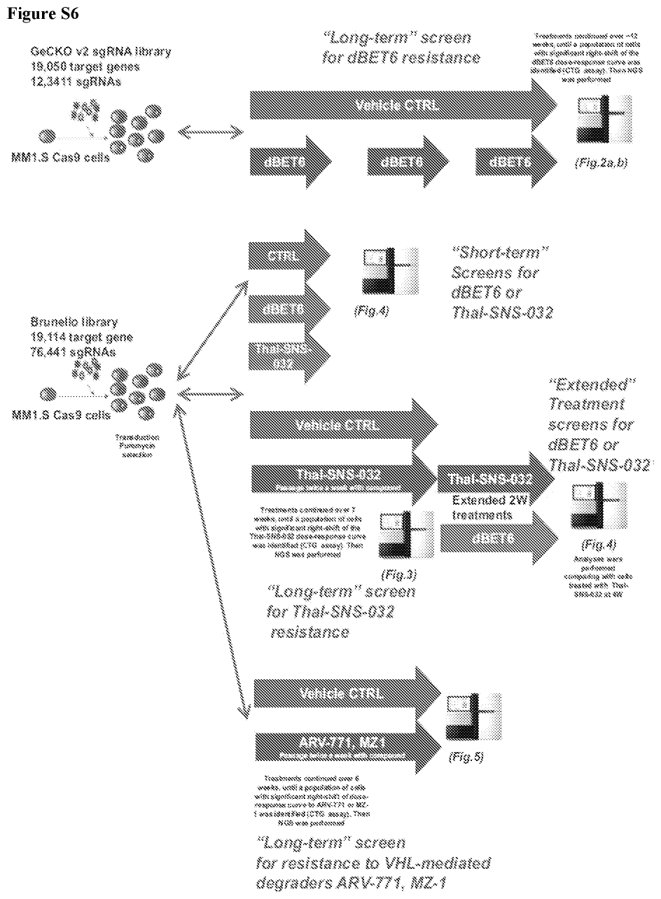

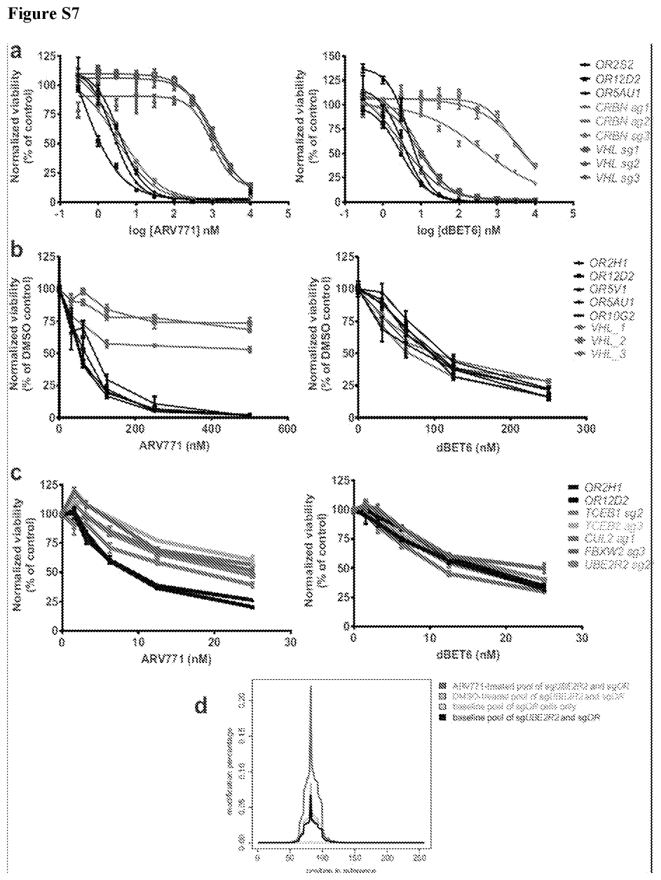

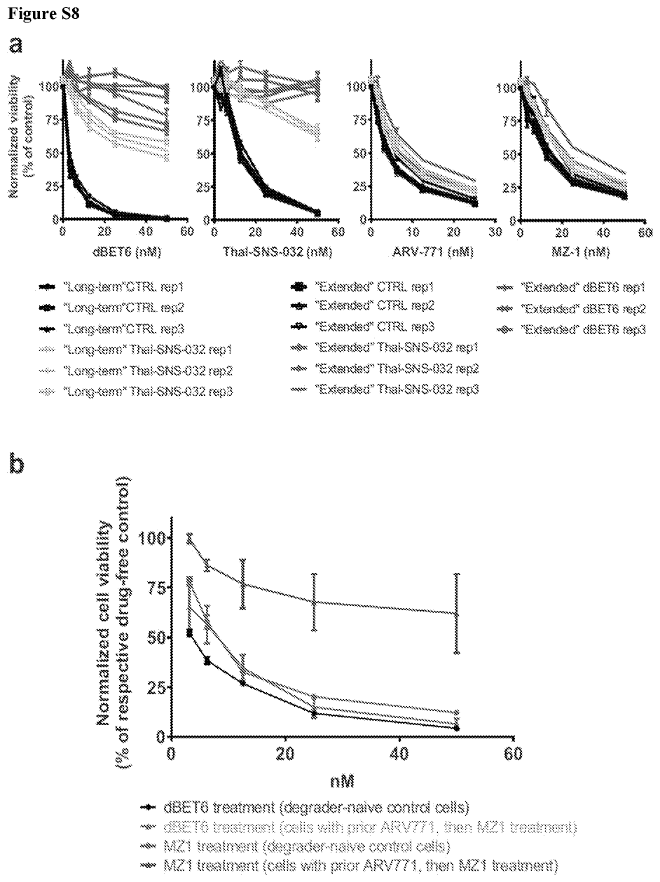

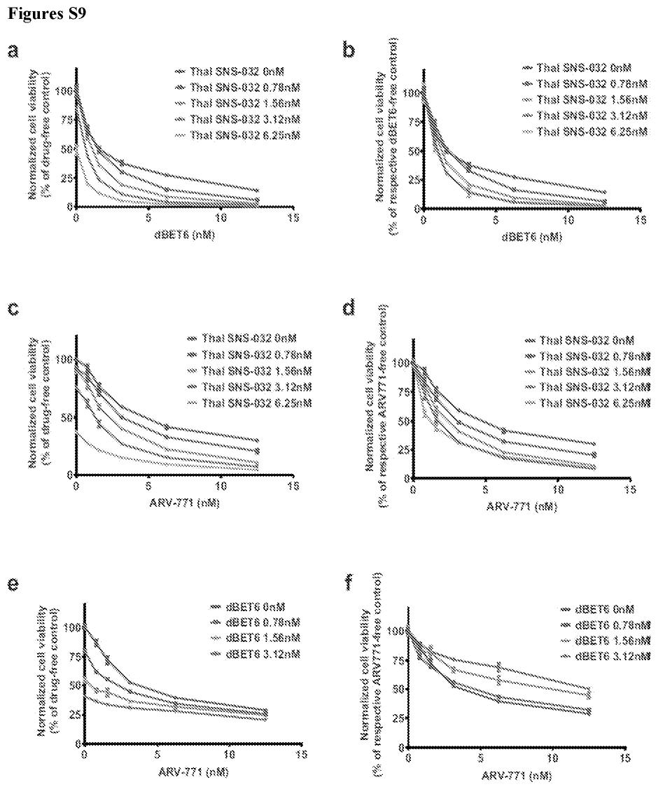

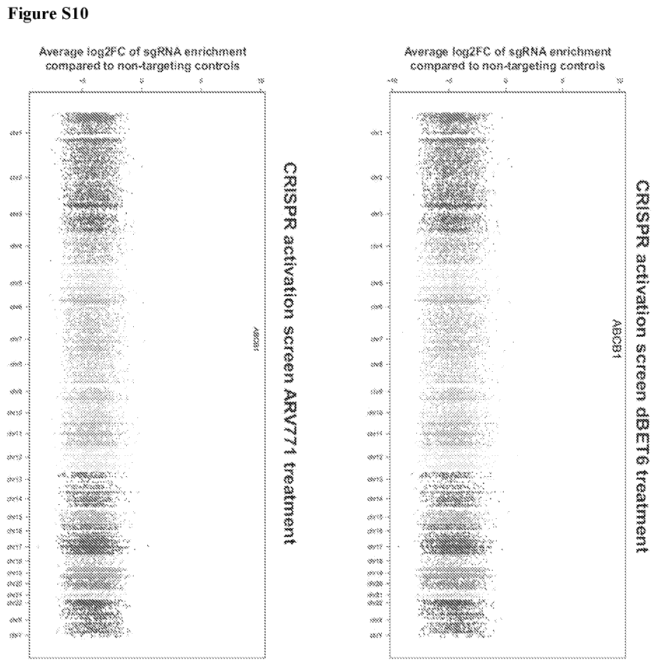

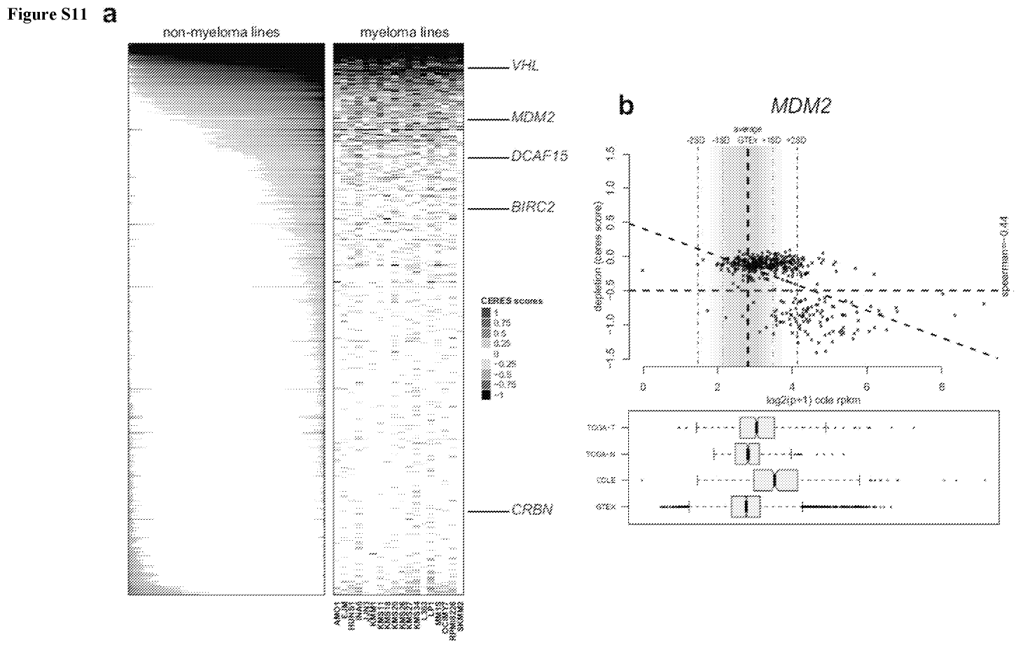

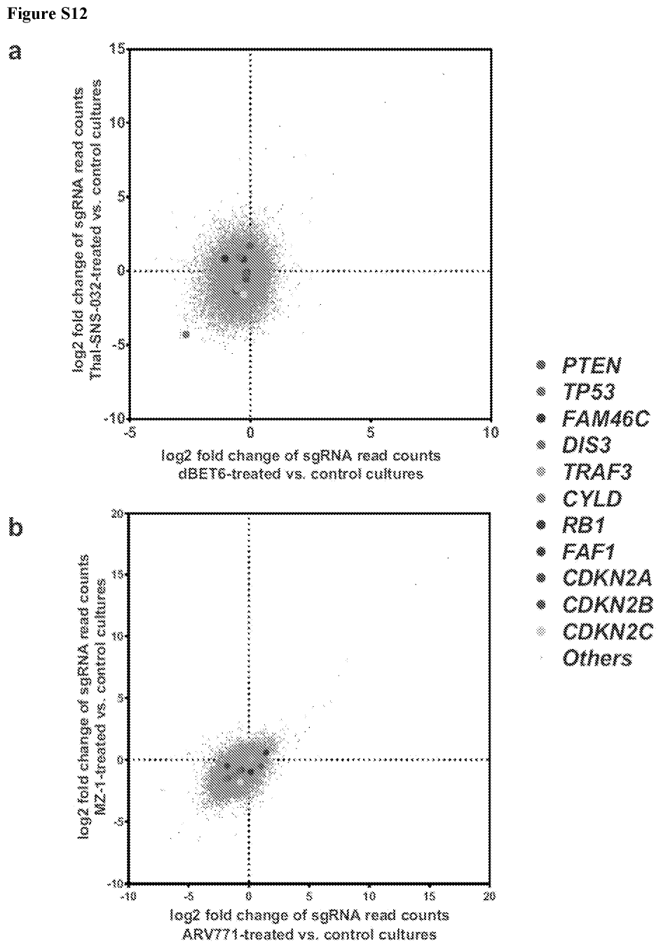

So far only a few (e.g. CRBN, VHL, MDM2 (Schneekloth et al., 2008), DCAF15 (Han et al., 2017; Uehara et al., 2017) and BIRC2 (Ohoka et al., 2018)) of the ˜600 known or presumed E3 ligases (Medvar et al., 2016; Nguyen et al., 2016) have been leveraged for the generation of PROTAC molecules, and most of them have not yet been formally examined for such a role. Given our observation that the redundant role of CRBN on tumor cell lines may influence the patterns and dynamics of response vs. resistance to CRBN-mediated degraders, we examined the dependency landscape of known E3 ligases across a broad range of neoplasias, based on results of genome-scale CRISPR essentiality screens in vitro. As shown in FIG. S11a, human tumor cell lines include a spectrum of E3 ligases whose CRISPR knock-out suppresses in vitro growth for the large majority (e.g. VHL) or sizeable subsets (e.g. MDM2, BIRC2, DCAF15) of human cancer cell lines, as well as other E3 ligases which, similarly to CRBN, are universally redundant for in vitro cell viability and proliferation. For degraders whose cognate E3 ligase and target protein are both broadly expressed in normal tissues, toxicities may conceivably ensue. We thus examined the landscape of E3 ligase expression vs. dependency in different tumor types, while also considering the distribution of E3 ligase transcript expression across a broad range of normal tissues, based on the GTEX database. We specifically sought to identify E3 ligases whose transcript levels in cancer cell lines are frequently higher than the majority of normal tissues. We indeed identified a series of E3 ligases for which >25% of lines expressing transcript levels higher than the average+2SD of expression in normal tissues, based on the GTEX database; and then examined for each E3 ligase, if a large percentage of their “high expressors” cell lines also depend on that same E3 ligase for in vitro proliferation and survival. We therefore identified E3 ligases, outlined in FIG. 7a-c, with frequent co-occurrence of essentiality and expression levels higher than the bulk of the distribution of transcription levels for normal tissues: these E3 ligases include VHL, other genes with well-known roles in tumor cell proliferation or survival (e.g. several members of the anaphase promoting complex/cyclosome (APC/C), a cell cycle-regulated E3 ubiquitin ligase that controls progression through mitosis and the G1 phase of the cell cycle (Turnell et al., 2005)), as well as several other E3 ligases (e.g. KCMF1, RNF4) which, to our knowledge, have not been extensively examined as candidates for potential design of PROTACs, but warrant further consideration, in view of their patterns of expression and essentiality for tumor cells. It is notable that, in the cell line panel examined, MDM2 also appears as an E3 ligase with high occurrence of essentiality among the “high expressor” cell lines (Figure S11b). This relationship predominantly applies to p53-wild-type cell lines (FIG. 7c,d), consistent with the role of MDM2 as an E3 ligase for p53.

Degronimids (Bondeson et al., 2017; Lebraud and Heightman, 2017; Lu et al., 2015; Olson et al., 2018; Robb et al., 2017; Winter et al., 2015; Winter et al., 2017; Wurz et al., 2017) and other heterobifunctional pharmacological “degraders” (Gadd et al., 2017; Gechijian et al., 2018; Han et al., 2017; Ohoka et al., 2018; Schneekloth et al., 2008; Uehara et al., 2017) are designed to deplete, rather than simply inhibit, the action of a therapeutic target. This property can be therapeutically advantageous in many contexts. For instance, the compensatory increase in BRD4 protein levels in tumor cells after treatment with BBIs could limit the therapeutic potential of their intermittent administration (Lu et al., 2015), and therefore “degraders” against BRD4 may improve on the modest clinical effects of first generation BBIs (Amorim et al., 2016). Our current study extended observations of potent in vitro antitumor activity of degraders against BET bromodomain proteins, CDK9 or other targets (Gadd et al., 2017; Lebraud et al., 2016; Lu et al., 2015; Saenz et al., 2017; Winter et al., 2015; Winter et al., 2017; Zengerle et al., 2015; Zhang et al., 2018; Zhou et al., 2017, 2018); and observed that CRBN-mediated degradation of BET bromodomain proteins exhibited in vitro activity even against MM cells resistant to other clinically used agents; or against pools of MM cells which had survived treatment with JQ1 or bortezomib in the context of genome-scale CRISPR-based gene editing studies.

To obtain an unbiased assessment of candidate genes regulating tumor cell responses to pharmacological “degraders”, we performed genome-scale CRISPR/Cas9-based gene editing studies in MM.1S cells treated with CRBN-mediated degraders of BET bromodomain proteins (dBET6) or CDK9 (Thal-SNS-032); or with VHL-mediated degraders of BET bromodomain proteins (ARV-771 or MZ-1). Surprisingly, among the top individual LOF events conferring resistance to these degraders, we identified genes which did not represent a compensatory mechanism or “work-around” the loss of BET domain proteins or CDK9; but rather the dysregulation of the degradation machinery itself.

Indeed, the top “hits” emerging from the CRISPR screens for both CRBN-mediated degraders, against either BRD4/3/2 or CDK9, were CRBN itself, and, to a quantitatively lesser extent, other members (e.g. DDB1, GPS1, UBE2G1) or regulators (e.g. COP9 signalosome genes, such as COPS7B, COPS7A, COPS8) of the cullin 4A-RING-CRBN E3 ubiquitin ligase (CRL) complex (CRL4CRBN) that catalyzes the ubiquitination of the respective target(s) of each degronimid. These observations are consistent with other CRISPR studies of our groups (Sievers et al., 2018) on candidate resistance genes to lenalidomide and pomalidomide, the prototypical “degronimids” which cause degradation of IKZF1/IKF3. Similarly, in the CRISPR knockout studies with two VHL-mediated degraders against BRD2/3/4, the top “hits” were components or regulators of the CRL2VHL complex, including CUL2 and VHL themselves, and, to a lesser extent, genes such as TCEB1/TCEB2 (elongin B/C), RBX1, UBE2R2, and the COP9 signalosome genes COPS7B and COPS8. It is notable that LOF events recurrently detected in MM patients and typically associated with high-risk MM (e.g. for TP53, P TEN, negative regulators of cell cycle, etc.) (Walker et al., 2015) are not enriched among “degrader”-resistant cells (FIG. S12 and data not shown), suggesting that this class of compounds may exhibit activity against tumor cells with prognostically adverse genetic features.

While the pathways or groups of genes which regulate tumor cell resistance have striking functional overlap for CRBN- vs. VHL-mediated degraders, the specific genes which represent these groups are different for the 2 classes of degraders that we studied. We interpret this result to reflect the fact that CRBN and VHL operate through different CRL complexes, with distinct composition and potentially different regulatory mechanisms. For instance, DDB1 and elongin BC (TCEB1 and TCEB2) preferentially associate with CRL complexes of CUL4A/B and CUL2, respectively (Kibel et al., 1995; Lonergan et al., 1998; Shiyanov et al., 1999) which explains why their LOF causes resistance to CRBN or VHL-mediated degraders, but not both. Even though LOF of the COP9 signalosome genes COPS7B and COPS8 confers resistance to both CRBN- and VHL-mediated degraders, this resistance effect is quantitatively more pronounced against the former group of degraders, suggesting a perhaps differential role of the COP9 signalosome or its individual components in the regulation of CRL2 vs. CRL4A complexes and potentially others.

These pathway-level similarities vs. individual gene-level differences in observed resistance mechanisms for CRBN- and VHL-mediated degraders have major potential implications for future clinical studies of E3-ligase mediated pharmacological degraders of oncoproteins. First, these results, combined with our phenotypic studies of concurrent or sequential administration of degraders from these 2 classes suggest that there is substantial cross-resistance between degraders targeting different oncoproteins through the same E3 ligase (e.g. as in our studies with CRBN-based degraders against BET bromodomain proteins or CDK9), but not between degraders operating through different E3 ligases and ideally, through different CRL complexes. Therefore, a main strategy to prevent or delay resistance to pharmacological degradation of oncoproteins is believed to involve the sequential/alternating use of degraders which target the same oncoprotein through different E3 ligases/CRL complexes, in order to maximally leverage the individual gene-level differences in the components and regulators of these complexes. Interestingly, in our study, concurrent administration of degraders operating through the same E3 ligase against different oncoproteins (e.g. CRBN-based degraders against BET bromodomain proteins or CDK9) or through different E3 ligases against different oncoproteins (e.g. CRBN-based degrader against CDK9 and VHL-based degrader against BET bromodomain proteins) leads to enhanced anti-tumor activity; in contrast, concurrent administration of degraders operating through different E3 ligases against the same oncoprotein (e.g. CRBN- and VHL-based degraders against BET bromodomain proteins) is associated with attenuation of the observed anti-tumor effect, perhaps indicative of a modified version of the so-called “hook” effect (Bondeson et al., 2015; Buckley et al., 2015; Burslem et al., 2017; Lu et al., 2015; Ohoka et al., 2018; Olson et al., 2018; Schiedel et al., 2017; Winter et al., 2015). The observations underlines the notion that the feasibility of using multiple pharmacological degraders for therapeutic applications may be highly contextual depending on a combination of factors, including, but not limited to the specific E3 ligase(s), oncoprotein targets and sequential vs. concurrent administration of these compounds. However, our finding that cross-resistance to pharmacological degradation of different oncoproteins through the same E3 ligase/CRL complex indicate that efforts to develop novel pharmacological degraders should be expanded to include as many different E3 ligases as possible from each type of CRL complexes.

Our observations also raise the hypothesis that inactivation of the COP9 signalosome could represent a common mechanism of decreased tumor cell response to pharmacological degraders operating through diverse E3 ligases and even different CRL complexes. One strategy to neutralize such a potential role of COP9 signalosome inactivation could be through combined administration of degraders with selective pharmacological activators of the COP9 signalosome. However, to our knowledge, compounds from this latter group have not been reported. Another approach could involve systematic characterization of the molecular vulnerabilities which might be selectively present in tumor cells with LOF of the COP9 signalosome vs. their wild-type counterparts, in order to design “synthetic lethal” strategies to hopefully delay or prevent emergence of tumor cell clones with COP9 signalosome inactivation and resistance to pharmacological degraders.

How the COP9 signalosome function may alter E3 ligase activity and hence the antitumor activity of degraders is not well understood. Neddylation of the cullin moiety of a CRL complex by the NEDD8-activating enzyme (NAE1) alters the conformation of the complex, allows the transfer of ubiquitin within the CRL complex from the E2 ubiquitin conjugating enzyme to the E3 ligase substrate and is thus considered to promote ubiquitination of the target of the CRL complex. The COP9 signalosome catalyzes the de-neddylation of the cullin moiety, promoting disassembly of the CRL complex and the re-assembly of new CRL complexes (Lydeard et al., 2013). This dynamic cycle of assembly, disassembly and remodeling of CRLs allows cells to leverage a relatively limited number of cullins and RING proteins to sustain, within a given period of time, the activity of a much larger number of assembled CRL complexes with different substrate specificities (Lydeard et al., 2013). Therefore, although COP9 signalosome-mediated de-neddylation of cullins should in principle suppress CRL function, it was instead reported that inhibition of COP9 signalosome (or CAND1) may “paradoxically” inactivate CRL function by disrupting the dynamic cycle of assembly, disassembly and remodeling of CRLs (Dubiel, 2009). It has also been reported that when substrates of the E3 ligase of a given CRL complex are not available, then the E3 ligase/CRL has the propensity to auto-ubiquitinate and thus accelerate its own degradation and LOF (Fischer et al., 2011), but that the COP9 signalosome complex prevents such auto-ubiquitination (Hotton and Callis, 2008). It is thus plausible that LOF of COP9 signalosome genes may confer degrader resistance by de-repressing the auto-ubiquitination of E3 ligases or potentially other components of the CRL complex, after an initial hyperactivation of CRLs (Hotton and Callis, 2008). Further studies will have to address whether LOF of signalosome genes confers decreased response to pharmacological degraders by disrupting the dynamic cycle of CRLs vs. by de-repressing the auto-ubiquitination of E3 ligases; and whether this mechanism applies to all or only some CRLs.

Our studies with CRBN- vs. VHL-mediated degraders revealed an interesting quantitative difference regarding these two E3 ligases: LOF for CRBN was consistently the top enriched “hit” in all configurations of our degronimid resistance studies, while LOF for VHL was not as highly selected for in either study of VHL-mediated degraders and exhibited 4-8 fold lower sgRNA enrichment compared to the top hit, CUL2. One explanation for such differences could be that the magnitude of sgRNA enrichment for a given gene may represent an aggregate effect of the “fitness benefit vs. cost” associated with LOF for that gene. For instance, if LOF of a gene suppresses proliferation of tumor cells in the absence of the degrader, but protects them against its cytotoxic effects, these two distinct properties confer respectively a “fitness cost” and a “fitness benefit”, a concept also observed for mechanisms of resistance to other therapeutics (e.g. (Tzoneva et al., 2018)). CRBN is universally dispensable for the proliferation and survival of human cancer cell lines in vitro and therefore it is plausible that its loss confers minimal fitness cost, as it protects tumor cells from degronimids while conferring no disadvantage in cell growth. By contrast, and for most human cancer cell lines (with the exception of renal cell carcinoma), LOF of VHL impairs tumor cell growth (FIG. 6 and (Bindra et al., 2002; Gamper et al., 2012; Welford et al., 2010; Young et al., 2008)) and therefore protects a tumor cell from the degrader, but also renders it less “fit”, which may explain why cells harboring sgRNAs against VHL will be less abundant in our genome-scale studies with both VHL-based degraders. These findings suggest that it may be prudent to design degrader compounds that act through ubiquitin ligase complexes whose function contribute to cell growth, but not ones that are redundant or act as tumor suppressors. In this latter case, LOF mutations of the ubiquitin ligase complex in question would lead to both tumor cell growth and resistance to the degronimid. Genome-scale screens against large panels of cancer cell lines can inform the selection of appropriate ligase complexes for design of pharmacological degraders.

Accordingly, to understand which E3 ligases represent good candidates for development of new pharmacological degraders, we surveyed E3 ligase expression in cancer cell lines. Several E3 ligases were identified whose expression in large subsets of human cancer cell lines was >2 standard deviations above the average expression in normal tissues and were required for growth of most of these cell lines. These included VHL, MDM2 (for p53 wild-type cell lines), components of the cell cycle regulated E3 ligase APC/C, and several other known (e.g. KCMF1, RNF4) or presumed E3 ligases not yet exploited for degrader design. The recent development of CRBN-mediated degronimids and other heterobifunctional degraders against different oncoproteins (Gechijian et al., 2018; Nabet et al., 2018; Olson et al., 2018; Winter et al., 2015; Winter et al., 2017; Xu et al., 2018) has offered new chemical probes to target key proteins in cancer by leveraging a relatively limited set of E3 ligase binders. Our study indicates that loss of function mutations that encourage resistance to degraders prevent the actual degradation of the target oncoprotein, rather than enable adaptation to its loss. Therefore, to prevent resistance to oncoprotein degraders, the development of more heterobifunctional compounds that utilize a variety of CRL complexes must be pursued. Empirical testing in vitro and importantly in vivo models of cancer will help inform whether such molecules would be best used in simultaneously or in sequential or alternating therapy.

### REFERENCES

Amorim, S., Stathis, A., Gleeson, M., Iyengar, S., Magarotto, V., Leleu, X., Morschhauser, F., Karlin, L., Broussais, F., Rezai, K., et al. (2016). Bromodomain inhibitor OTX015 in patients with lymphoma or multiple myeloma: a dose-escalation, open-label, pharmacokinetic, phase 1 study. Lancet Haematol 3, e196-204.
Bindra, R. S., Vasselli, J. R., Stearman, R., Linehan, W. M., and Klausner, R. D. (2002). VHL-mediated hypoxia regulation of cyclin D1 in renal carcinoma cells. Cancer Res 62, 3014-3019.
Bondeson, D. P., Mares, A., Smith, I. E., Ko, E., Campos, S., Miah, A. H., Mulholland, K. E., Routly, N., Buckley, D. L., Gustafson, J. L., et al. (2015). Catalytic in vivo protein knockdown by small-molecule PROTACs. Nat Chem Biol 11, 611-617.
Bondeson, D. P., Smith, B. E., Burslem, G. M., Buhimschi, A. D., Hines, J., Jaime-Figueroa, S., Wang, J., Hamman, B. D., Ishchenko, A., and Crews, C. M. (2017). Lessons in PROTAC Design from Selective Degradation with a Promiscuous Warhead. Cell Chem Biol. Buckley, D. L., Raina, K., Darricarrere, N., Hines, J., Gustafson, J. L., Smith, I. E., Miah, A. H., Harling, J. D., and Crews, C. M. (2015). HaloPROTACS: Use of Small Molecule PROTACs to Induce Degradation of HaloTag Fusion Proteins. ACS Chem Biol 10, 1831-1837.
Burslem, G. M., Smith, B. E., Lai, A. C., Jaime-Figueroa, S., McQuaid, D. C., Bondeson, D. P., Toure, M., Dong, H., Qian, Y., Wang, J., et al. (2017). The Advantages of Targeted Protein Degradation Over Inhibition: An RTK Case Study. Cell Chem Biol.
Delmore, J. E., Issa, G. C., Lemieux, M. E., Rahl, P. B., Shi, J., Jacobs, H. M., Kastritis, E., Gilpatrick, T., Paranal, R. M., Qi, J., et al. (2011). BET bromodomain inhibition as a therapeutic strategy to target c-Myc. Cell 146, 904-917.
Dubiel, W. (2009). Resolving the CSN and CAND1 paradoxes. Mol Cell 35, 547-549. Fischer, E. S., Scrima, A., Bohm, K., Matsumoto, S., Lingaraju, G. M., Faty, M., Yasuda, T., Cavadini, S., Wakasugi, M., Hanaoka, F., et al. (2011). The molecular basis of CRL4DDB2/CSA ubiquitin ligase architecture, targeting, and activation. Cell 147, 1024-1039.
Foulk, B., Schaffer, M., Gross, S., Rao, C., Smirnov, D., Connelly, M. C., Chaturvedi, S., Reddy, M., Brittingham, G., Mata, M., et al. (2018). Enumeration and characterization of circulating multiple myeloma cells in patients with plasma cell disorders. Br J Haematol 180, 71-81.
Gadd, M. S., Testa, A., Lucas, X., Chan, K. H., Chen, W., Lamont, D. J., Zengerle, M., and Ciulli, A. (2017). Structural basis of PROTAC cooperative recognition for selective protein degradation. Nat Chem Biol 13, 514-521.
Gamper, A. M., Qiao, X., Kim, J., Zhang, L., DeSimone, M. C., Rathmell, W. K., and Wan, Y. (2012). Regulation of KLF4 turnover reveals an unexpected tissue-specific role of pVHL in tumorigenesis. Mol Cell 45, 233-243.
Gandhi, A. K., Mendy, D., Waldman, M., Chen, G., Rychak, E., Miller, K., Gaidarova, S., Ren, Y., Wang, M., Breider, M., et al. (2014). Measuring cereblon as a biomarker of response or resistance to lenalidomide and pomalidomide requires use of standardized reagents and understanding of gene complexity. Br J Haematol 164, 233-244.
Gechijian, L. N., Buckley, D. L., Lawlor, M. A., Reyes, J. M., Paulk, J., Ott, C. J., Winter, G. E., Erb, M. A., Scott, T. G., Xu, M., et al. (2018). Functional TRIM24 degrader via conjugation of ineffectual bromodomain and VHL ligands. Nat Chem Biol 14, 405-412.
Han, T., Goralski, M., Gaskill, N., Capota, E., Kim, J., Ting, T. C., Xie, Y., Williams, N. S., and Nijhawan, D. (2017). Anticancer sulfonamides target splicing by inducing RBM39 degradation via recruitment to DCAF15. Science 356.
Heintel, D., Rocci, A., Ludwig, H., Bolomsky, A., Caltagirone, S., Schreder, M., Pfeifer, S., Gisslinger, H., Zojer, N., Jager, U., et al. (2013). High expression of cereblon (CRBN) is associated with improved clinical response in patients with multiple myeloma treated with lenalidomide and dexamethasone. Br J Haematol 161, 695-700.
Hotton, S. K., and Callis, J. (2008). Regulation of cullin RING ligases. Annu Rev Plant Biol 59, 467-489.
Kibel, A., Iliopoulos, O., DeCaprio, J. A., and Kaelin, W. G., Jr. (1995). Binding of the von Hippel-Lindau tumor suppressor protein to Elongin B and C. Science 269, 1444-1446.
Kowalski, J., Dwivedi, B., Newman, S., Switchenko, J. M., Pauly, R., Gutman, D. A., Arora, J., Gandhi, K., Ainslie, K., Doho, G., et al. (2016). Gene integrated set profile analysis: a context-based approach for inferring biological endpoints. Nucleic acids research 44, e69.
Kronke, J., Udeshi, N. D., Narla, A., Grauman, P., Hurst, S. N., McConkey, M., Svinkina, T., Heckl, D., Comer, E., Li, X., et al. (2014). Lenalidomide causes selective degradation of IKZF1 and IKZF3 in multiple myeloma cells. Science 343, 301-305.
Lebraud, H., and Heightman, T. D. (2017). Protein degradation: a validated therapeutic strategy with exciting prospects. Essays Biochem 61, 517-527.
Lebraud, H., Wright, D. J., Johnson, C. N., and Heightman, T. D. (2016). Protein Degradation by In-Cell Self-Assembly of Proteolysis Targeting Chimeras. ACS Cent Sci 2, 927-934.
Lonergan, K. M., Iliopoulos, O., Ohh, M., Kamura, T., Conaway, R. C., Conaway, J. W., and Kaelin, W. G., Jr. (1998). Regulation of hypoxia-inducible mRNAs by the von Hippel-Lindau tumor suppressor protein requires binding to complexes containing elongins B/C and Cul2. Molecular and cellular biology 18, 732-741.
Loven, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of super-enhancers. Cell 153, 320-334.
Lu, G., Middleton, R. E., Sun, H., Naniong, M., Ott, C. J., Mitsiades, C. S., Wong, K. K., Bradner, J. E., and Kaelin, W. G., Jr. (2014). The myeloma drug lenalidomide promotes the cereblon-dependent destruction of Ikaros proteins. Science 343, 305-309.
Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chem Biol 22, 755-763.
Lydeard, J. R., Schulman, B. A., and Harper, J. W. (2013). Building and remodelling Cullin-RING E3 ubiquitin ligases. EMBO Rep 14, 1050-1061.
McMillin, D. W., Delmore, J., Negri, J. M., Vanneman, M., Koyama, S., Schlossman, R. L., Munshi, N.C., Laubach, J., Richardson, P. G., Dranoff, G., et al. (2012). Compartment-Specific Bioluminescence Imaging platform for the high-throughput evaluation of antitumor immune function. Blood 119, e131-138.
McMillin, D. W., Delmore, J., Weisberg, E., Negri, J. M., Geer, D. C., Klippel, S., Mitsiades, N., Schlossman, R. L., Munshi, N.C., Kung, A. L., et al. (2010). Tumor cell-specific bioluminescence platform to identify stroma-induced changes to anticancer drug activity. Nature medicine 16, 483-489.
Medvar, B., Raghuram, V., Pisitkun, T., Sarkar, A., and Knepper, M. A. (2016). Comprehensive database of human E3 ubiquitin ligases: application to aquaporin-2 regulation. Physiological genomics 48, 502-512.
Miller, A., Asmann, Y., Cattaneo, L., Braggio, E., Keats, J., Auclair, D., Lonial, S., Russell, S. J., and Stewart, A. K. (2017). High somatic mutation and neoantigen burden are correlated with decreased progression-free survival in multiple myeloma. Blood cancer journal 7, e612.
Nabet, B., Roberts, J. M., Buckley, D. L., Paulk, J., Dastjerdi, S., Yang, A., Leggett, A. L., Erb, M. A., Lawlor, M. A., Souza, A., et al. (2018). The dTAG system for immediate and target-specific protein degradation. Nat Chem Biol 14, 431-441.
Nguyen, V. N., Huang, K. Y., Weng, J. T., Lai, K. R., and Lee, T. Y. (2016). UbiNet: an online resource for exploring the functional associations and regulatory networks of protein ubiquitylation. Database (Oxford) 2016.
Ohoka, N., Morita, Y., Nagai, K., Shimokawa, K., Ujikawa, O., Fujimori, I., Ito, M., Hayase, Y., Okuhira, K., Shibata, N., et al. (2018). Derivatization of inhibitor of apoptosis protein (IAP) ligands yields improved inducers of estrogen receptor alpha degradation. J Biol Chem 293, 6776-6790.
Olson, C. M., Jiang, B., Erb, M. A., Liang, Y., Doctor, Z. M., Zhang, Z., Zhang, T., Kwiatkowski, N., Boukhali, M., Green, J. L., et al. (2018). Pharmacological perturbation of CDK9 using selective CDK9 inhibition or degradation. Nat Chem Biol 14, 163-170.
Raina, K., Lu, J., Qian, Y., Altieri, M., Gordon, D., Rossi, A. M., Wang, J., Chen, X., Dong, H., Siu, K., et al. (2016). PROTAC-induced BET protein degradation as a therapy for castration-resistant prostate cancer. Proc Natl Acad Sci USA 113, 7124-7129.
Robb, C. M., Contreras, J. I., Kour, S., Taylor, M. A., Abid, M., Sonawane, Y. A., Zahid, M., Murry, D. J., Nataraj an, A., and Rana, S. (2017). Chemically induced degradation of CDK9 by a proteolysis targeting chimera (PROTAC). Chem Commun (Camb) 53, 7577-7580.
Saenz, D. T., Fiskus, W., Qian, Y., Manshouri, T., Rajapakshe, K., Raina, K., Coleman,
K. G., Crew, A. P., Shen, A., Mill, C. P., et al. (2017). Novel BET protein proteolysis-targeting chimera exerts superior lethal activity than bromodomain inhibitor (BETi) against post-myeloproliferative neoplasm secondary (s) AML cells. Leukemia 31, 1951-1961.
Schiedel, M., Herp, D., Hammelmann, S., Swyter, S., Lehotzky, A., Robaa, D., Olah, J., Ovadi, J., Sippl, W., and Jung, M. (2017). Chemically Induced Degradation of Sirtuin 2 (Sirt2) by a Proteolysis Targeting Chimera (PROTAC) Based on Sirtuin Rearranging Ligands (SirReals). J Med Chem.
Schneekloth, A. R., Pucheault, M., Tae, H. S., and Crews, C. M. (2008). Targeted intracellular protein degradation induced by a small molecule: En route to chemical proteomics. Bioorg Med Chem Lett 18, 5904-5908.
Shiyanov, P., Nag, A., and Raychaudhuri, P. (1999). Cullin 4A associates with the UV-damaged DNAbinding protein DDB. J Biol Chem 274, 35309-35312.
Sievers, Q. L., Gasser, J. A., Cowley, G. S., Fischer, E. S., and Ebert, B. L. (2018). Genome-wide screen identifies cullin-RING ligase machinery required for lenalidomide-dependent CRL4(CRBN) activity. Blood 132, 1293-1303.
Sun, B., Fiskus, W., Qian, Y., Rajapakshe, K., Raina, K., Coleman, K. G., Crew, A. P., Shen, A., Saenz, D. T., Mill, C. P., et al. (2017). BET protein proteolysis targeting chimera (PROTAC) exerts potent lethal activity against mantle cell lymphoma cells. Leukemia.
Turnell, A. S., Stewart, G. S., Grand, R. J., Rookes, S. M., Martin, A., Yamano, H., Elledge, S. J., and Gallimore, P. H. (2005). The APC/C and CBP/p300 cooperate to regulate transcription and cell-cycle progression. Nature 438, 690-695.
Tzoneva, G., Dieck, C. L., Oshima, K., Ambesi-Impiombato, A., Sanchez-Martin, M., Madubata, C. J., Khiabanian, H., Yu, J., Waanders, E., Iacobucci, I., et al. (2018). Clonal evolution mechanisms in NT5C2 mutant-relapsed acute lymphoblastic leukaemia. Nature 553, 511-514.
Uehara, T., Minoshima, Y., Sagane, K., Sugi, N. H., Mitsuhashi, K. O., Yamamoto, N., Kamiyama, H., Takahashi, K., Kotake, Y., Uesugi, M., et al. (2017). Selective degradation of splicing factor CAPERalpha by anticancer sulfonamides. Nat Chem Biol 13, 675-680.
Walker, B. A., Boyle, E. M., Wardell, C. P., Murison, A., Begum, D. B., Dahir, N. M., Proszek, P. Z., Johnson, D. C., Kaiser, M. F., Melchor, L., et al. (2015). Mutational Spectrum, Copy Number Changes, and Outcome: Results of a Sequencing Study of Patients With Newly Diagnosed Myeloma. Journal of clinical oncology: official journal of the American Society of Clinical Oncology 33, 3911-3920.
Welford, S. M., Dorie, M. J., Li, X., Haase, V. H., and Giaccia, A. J. (2010). Renal oxygenation suppresses VHL loss-induced senescence that is caused by increased sensitivity to oxidative stress. Molecular and cellular biology 30, 4595-4603.
Winter, G. E., Buckley, D. L., Paulk, J., Roberts, J. M., Souza, A., Dhe-Paganon, S., and Bradner, J. E. (2015). Drug Development. Phthalimide conjugation as a strategy for in vivo target protein degradation. Science 348, 1376-1381.
Winter, G. E., Mayer, A., Buckley, D. L., Erb, M. A., Roderick, J. E., Vittori, S., Reyes, J. M., di Iulio, J., Souza, A., Ott, C. J., et al. (2017). BET Bromodomain Proteins Function as Master Transcription Elongation Factors Independent of CDK9 Recruitment. Mol Cell 67, 5-18 e19.
Wurz, R. P., Dellamaggiore, K., Dou, H., Javier, N., Lo, M. C., McCarter, J. D., Mohl, D., Sastri, C., Lipford, J. R., and Cee, V. J. (2017). A “Click Chemistry Platform” for the Rapid Synthesis of Bispecific Molecules for Inducing Protein Degradation. J Med Chem.
Xu, L., Chen, Y., Mayakonda, A., Koh, L., Chong, Y. K., Buckley, D. L., Sandanaraj, E., Lim, S. W., Lin, R. Y., Ke, X. Y., et al. (2018). Targetable BET proteins- and E2F1-dependent transcriptional program maintains the malignancy of glioblastoma. Proc Natl Acad Sci USA.
Young, A. P., Schlisio, S., Minamishima, Y. A., Zhang, Q., Li, L., Grisanzio, C., Signoretti, S., and Kaelin, W. G., Jr. (2008). VHL loss actuates a HIF-independent senescence programme mediated by Rb and p400. Nature cell biology 10, 361-369.
Zengerle, M., Chan, K. H., and Ciulli, A. (2015). Selective Small Molecule Induced Degradation of the BET Bromodomain Protein BRD4. ACS Chem Biol 10, 1770-1777.
Zhang, X., Lee, H. C., Shirazi, F., Baladandayuthapani, V., Lin, H., Kuiatse, I., Wang, H., Jones, R. J., Berkova, Z., Singh, R. K., et al. (2018). Protein targeting chimeric molecules specific for bromodomain and extra-terminal motif family proteins are active against pre-clinical models of multiple myeloma. Leukemia.
Zhou, B., Hu, J., Xu, F., Chen, Z., Bai, L., Fernandez-Salas, E., Lin, M., Liu, L., Yang, C. Y., Zhao, Y., et al. (2017). Discovery of a Small-Molecule Degrader of Bromodomain and Extra-Terminal (BET) Proteins with Picomolar Cellular Potencies and Capable of Achieving Tumor Regression. J Med Chem.
Zhou, B., Hu, J., Xu, F., Chen, Z., Bai, L., Fernandez-Salas, E., Lin, M., Liu, L., Yang, C. Y., Zhao, Y., et al. (2018). Discovery of a Small-Molecule Degrader of Bromodomain and Extra-Terminal (BET) Proteins with Picomolar Cellular Potencies and Capable of Achieving Tumor Regression. J Med Chem 61, 462-481.
Zhu, Y. X., Braggio, E., Shi, C. X., Bruins, L. A., Schmidt, J. E., Van Wier, S., Chang, X. B., Bjorklund, C. C., Fonseca, R., Bergsagel, P. L., et al. (2011). Cereblon expression is required for the antimyeloma=activity of lenalidomide and pomalidomide. Blood 118, 4771-4779.
Zuber, J., Shi, J., Wang, E., Rappaport, A. R., Herrmann, H., Sison, E. A., Magoon, D., Qi, J., Blatt, K., Wunderlich, M., et al. (2011). RNAi screen identifies Brd4 as a therapeutic target in acute myeloid leukaemia. Nature 478, 524-528.
Delmore, J. E., Issa, G. C., Lemieux, M. E., Rahl, P. B., Shi, J., Jacobs, H. M., Kastritis, E., Gilpatrick, T., Paranal, R. M., Qi, J., et al. (2011). BET bromodomain inhibition as a therapeutic strategy to target c-Myc. Cell 146, 904-917.
Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize off-target effects of CRISPR-Cas9. Nature biotechnology 34, 184-191.
Kolde, R., Laur, S., Adler, P., and Vilo, J. (2012). Robust rank aggregation for gene list integration and meta-analysis. Bioinformatics 28, 573-580.
Li, J., Zhao, W., Akbani, R., Liu, W., Ju, Z., Ling, S., Vellano, C. P., Roebuck, P., Yu, Q., Eterovic, A. K., et al. (2017). Characterization of Human Cancer Cell Lines by Reverse-phase Protein Arrays. Cancer Cell 31, 225-239.
Li, W., Xu, H., Xiao, T., Cong, L., Love, M. I., Zhang, F., Irizarry, R. A., Liu, J. S., Brown, M., and Liu, X. S. (2014). MAGeCK enables robust identification of essential genes from genome-scale CRISPR/Cas9 knockout screens. Genome Biol 15, 554.
Matthews, G. M., Mehdipour, P., Cluse, L. A., Falkenberg, K. J., Wang, E., Roth, M., Santoro, F., Vidacs, E., Stanley, K., House, C. M., et al. (2015). Functional-genetic dissection of HDAC dependencies in mouse lymphoid and myeloid malignancies. Blood 126, 2392-2403.
McMillin, D. W., Delmore, J., Negri, J., Ooi, M., Klippel, S., Miduturu, C. V., Gray, N. S., Richardson, P. G., Anderson, K. C., Kung, A. L., and Mitsiades, C. S. (2011). Microenvironmental influence on pre-clinical activity of polo-like kinase inhibition in multiple myeloma: implications for clinical translation. PloS one 6, e20226.
McMillin, D. W., Delmore, J., Negri, J. M., Vanneman, M., Koyama, S., Schlossman, R. L., Munshi, N. C., Laubach, J., Richardson, P. G., Dranoff, G., et al. (2012a). Compartment-Specific Bioluminescence Imaging platform for the high-throughput evaluation of antitumor immune function. Blood 119, e131-138.
McMillin, D. W., Delmore, J., Weisberg, E., Negri, J. M., Geer, D. C., Klippel, S., Mitsiades, N., Schlossman, R. L., Munshi, N. C., Kung, A. L., et al. (2010). Tumor cell-specific bioluminescence platform to identify stroma-induced changes to anticancer drug activity. Nature medicine 16, 483-489.
McMillin, D. W., Jacobs, H. M., Delmore, J. E., Buon, L., Hunter, Z. R., Monrose, V., Yu, J., Smith, P. G., Richardson, P. G., Anderson, K. C., et al. (2012b). Molecular and cellular effects of NEDD8-activating enzyme inhibition in myeloma. Mol Cancer Ther 11, 942-951.
Meyers, R. M., Bryan, J. G., McFarland, J. M., Weir, B. A., Sizemore, A. E., Xu, H., Dharia, N. V., Montgomery, P. G., Cowley, G. S., Pantel, S., et al. (2017). Computational correction of copy number effect improves specificity of CRISPR-Cas9 essentiality screens in cancer cells. Nature genetics 49, 1779-1784.
Mitsiades, C. S., Ocio, E. M., Pandiella, A., Maiso, P., Gajate, C., Garayoa, M., Vilanova, D., Montero, J. C., Mitsiades, N., McMullan, C. J., et al. (2008). Aplidin, a marine organism-derived compound with potent antimyeloma activity in vitro and in vivo. Cancer research 68, 5216-5225.
Newbold, A., Matthews, G. M., Bots, M., Cluse, L. A., Clarke, C. J., Banks, K. M., Cullinane, C., Bolden, J. E., Christiansen, A. J., Dickins, R. A., et al. (2013). Molecular and biologic analysis of histone deacetylase inhibitors with diverse specificities. Mol Cancer Ther 12, 2709-2721.
Olson, C. M., Jiang, B., Erb, M. A., Liang, Y., Doctor, Z. M., Zhang, Z., Zhang, T., Kwiatkowski, N., Boukhali, M., Green, J. L., et al. (2018). Pharmacological perturbation of CDK9 using selective CDK9 inhibition or degradation. Nat Chem Biol 14, 163-170.
Ritchie, M. E., Phipson, B., Wu, D., Hu, Y., Law, C. W., Shi, W., and Smyth, G. K. (2015). Limma powers differential expression analyses for RNA-sequencing and microarray studies. Nucleic Acids Res 43, e47.
Sanjana, N. E., Shalem, O., and Zhang, F. (2014). Improved vectors and genome-wide libraries for CRISPR screening. Nature methods 11, 783-784.
Shalem, O., Sanjana, N. E., Hartenian, E., Shi, X., Scott, D. A., Mikkelson, T., Heckl, D., Ebert, B. L., Root, D. E., Doench, J. G., and Zhang, F. (2014). Genome-scale CRISPR-Cas9 knockout screening in human cells. Science 343, 84-87.
Wan, L., Wen, H., Li, Y., Lyu, J., Xi, Y., Hoshii, T., Joseph, J. K., Wang, X., Loh, Y. E., Erb, M. A., et al. (2017). ENL links histone acetylation to oncogenic gene expression in acute myeloid leukaemia. Nature 543, 265-269.
Wang, B., Krall, E. B., Aguirre, A. J., Kim, M., Widlund, H. R., Doshi, M. B., Sicinska, E., Sulahian, R., Goodale, A., Cowley, G. S., et al. (2017). ATXN1L, CIC, and ETS Transcription Factors Modulate Sensitivity to MAPK Pathway Inhibition. Cell Rep 18, 1543-1557.
Winter, G. E., Mayer, A., Buckley, D. L., Erb, M. A., Roderick, J. E., Vittori, S., Reyes, J. M., diIulio, J., Souza, A., Ott, C. J., et al. (2017). BET Bromodomain Proteins Function as Master Transcription Elongation Factors Independent of CDK9 Recruitment. Mol Cell 67, 5-18 e19.
Zhu, Y. X., Kortuem, K. M., and Stewart, A. K. (2013). Molecular mechanism of action of immunemodulatory drugs thalidomide, lenalidomide and pomalidomide in multiple myeloma. Leuk Lymphoma 54, 683-687.

TABLE S1
List of primers

| | | | | | | Rev. |
|---|---|---|---|---|---|---|
| | | | | | | comp. |
| Pri- | | TM | | GC | Bar- | bar- |
| mers: | | ° c. | Size | % | code | code |
| F1 | AATGGACTATCATATGCTTAC | 69.2 | 41 | 34.1 | | |
| | CGTAACTTGAAAGTATTTCG | | | | | |
| R1 | CTTTAGTTTGTATGTCTGTTG | 68.3 | 44 | 31.8 | | |
| | CTATTATGTCTACTATTCTTT | | | | | |
| | CC | | | | | |
| R21 | CAAGCAGAAGACGGCATACGA | 79.2 | 89 | 50.6 | CCTC | CAGA |
| | GATCCTCTCTG GTGACTGGA | | | | TCTG | GAGG |
| | GTTCAGACGTGTGCTCTTCCG | | | | | |
| | ATCTtctactattctttccct | | | | | |
| | gcact | | | | | |
| R22 | CAAGCAGAAGACGGCATACGA | 79.2 | 89 | 50.6 | CTAG | CGTA |
| | GATCTAGTACG GTGACTGGA | | | | TACG | CTAG |
| | GTTCAGACGTGTGCTCTTCCG | | | | | |
| | ATCTtctactattctttccct | | | | | |
| | gcact | | | | | |
| R23 | CAAGCAGAAGACGGCATACGA | 79.2 | 89 | 50.6 | TTCT | AGGC |
| | GATTTCTGCCT GTGACTGGA | | | | GCCT | AGAA |
| | GTTCAGACGTGTGCTCTTCCG | | | | | |
| | ATCTtctactattctttccct | | | | | |
| | gcact | | | | | |
| R24 | CAAGCAGAAGACGGCATACGA | 79.2 | 89 | 50.6 | GCTC | TCCT |
| | GATGCTCAGGA GTGACTGGA | | | | AGGA | GAGC |
| | GTTCAGACGTGTGCTCTTCCG | | | | | |
| | ATCTtctactattctttccct | | | | | |
| | gcact | | | | | |
| R25 | CAAGCAGAAGACGGCATACGA | 79.2 | 89 | 50.6 | AGGA | GGAC |
| | GATAGGAGTCC GTGACTGGA | | | | GTCC | TCCT |
| | GTTCAGACGTGTGCTCTTCCG | | | | | |
| | ATCTtctactattctttccct | | | | | |
| | gcact | | | | | |
| R26 | CAAGCAGAAGACGGCATACGA | 79.2 | 89 | 50.6 | CATG | TAGG |
| | GATCATGCCTA GTGACTGGA | | | | CCTA | CATG |
| | GTTCAGACGTGTGCTCTTCCG | | | | | |
| | ATCTtctactattctttccct | | | | | |
| | gcact | | | | | |
| R27 | CAAGCAGAAGACGGCATACGA | 79.2 | 89 | 50.6 | GTAG | CTCT |
| | GATGTAGAGAG GTGACTGGA | | | | AGAG | CTAC |
| | GTTCAGACGTGTGCTCTTCCG | | | | | |
| | ATCTtctactattctttccct | | | | | |
| | gcact | | | | | |
| R28 | CAAGCAGAAGACGGCATACGA | 79.2 | 89 | 50.6 | AACA | CCAT |
| | GATAACAATGGGTGACTGGAG | | | | ATGG | TGTT |
| | TTCAGACGTGTGCTCTTCCGA | | | | | |
| | TCTtctactattctttccctg | | | | | |
| | cact | | | | | |
| R29 | CAAGCAGAAGACGGCATACGA | 79.2 | 89 | 50.6 | AGCG | GCTA |
| | GATAGCGTAGCGTGACTGGAG | | | | TAGC | CGCT |
| | TTCAGACGTGTGCTCTTCCGA | | | | | |
| | TCTtctactattctttccctg | | | | | |
| | cact | | | | | |
| R210 | CAAGCAGAAGACGGCATACGA | 79.2 | 89 | 50.6 | CAGC | CGAG |
| | GATCAGCCTCGGTGACTGGAG | | | | CTCG | GCTG |
| | TTCAGACGTGTGCTCTTCCGA | | | | | |
| | TCTtctactattctttccctg | | | | | |
| | cact | | | | | |
| R211 | CAAGCAGAAGACGGCATACGA | 79.2 | 89 | 50.6 | AGTA | ACGC |
| | GATAGTAGCGTGTGACTGGAG | | | | GCGT | TACT |
| | TTCAGACGTGTGCTCTTCCGA | | | | | |
| | TCTtctactattctttccctg | | | | | |
| | cact | | | | | |
| R212 | CAAGCAGAAGACGGCATACGA | 79.2 | 89 | 50.6 | CAGT | ACTC |
| | GATCAGTGAGTGTGACTGGAG | | | | GAGT | ACTG |
| | TTCAGACGTGTGCTCTTCCGA | | | | | |
| | TCTtctactattctttccctg | | | | | |
| | cact | | | | | |
| R213 | CAAGCAGAAGACGGCATACGA | 79.2 | 89 | 50.6 | CGTA | TGAG |
| | GATCGTACTCAGTGACTGGAG | | | | CTCA | TACG |
| | TTCAGACGTGTGCTCTTCCGA | | | | | |
| | TCTtctactattctttccctg | | | | | |
| | cact | | | | | |
| R214 | CAAGCAGAAGACGGCATACGA | 79.2 | 89 | 50.6 | CTAC | CTGC |
| | GATCTACGCAGGTGACTGGAG | | | | GCAG | GTAG |
| | TTCAGACGTGTGCTCTTCCGA | | | | | |
| | TCTtctactattctttccctg | | | | | |
| | cact | | | | | |
| R215 | CAAGCAGAAGACGGCATACGA | 79.2 | 89 | 50.6 | GGAG | TAGT |
| | GATGGAGACTAGTGACTGGAG | | | | ACTA | CTCC |
| | TTCAGACGTGTGCTCTTCCGA | | | | | |
| | TCTtctactattctttccctg | | | | | |
| | cact | | | | | |
| R216 | CAAGCAGAAGACGGCATACGA | 79.2 | 89 | 50.6 | AGGT | CCTT |
| | GATAGGTAAGGGTGACTGGAG | | | | AAGG | ACCT |
| | TTCAGACGTGTGCTCTTCCGA | | | | | |
| | TCTtctactattctttccctg | | | | | |
| | cact | | | | | |
| R217 | CAAGCAGAAGACGGCATACGA | 79.2 | 89 | 50.6 | AACG | AATG |
| | GATAACGCATTGTGACTGGAG | | | | CATT | CGTT |
| | TTCAGACGTGTGCTCTTCCGA | | | | | |
| | TCTtctactattctttccctg | | | | | |
| | cact | | | | | |
| R218 | CAAGCAGAAGACGGCATACGA | 79.2 | 89 | 50.6 | ACAG | ATAC |
| | GATACAGGTATGTGACTGGAG | | | | GTAT | CTGT |
| | TTCAGACGTGTGCTCTTCCGA | | | | | |
| | TCTtctactattctttccctg | | | | | |
| | cact | | | | | |
| F2 | AATGATACGGCGACCACCGAG | 79.4 | 82 | 51.2 | | |
| | ATCTACACTCTTTCCCTACAC | | | | | |
| | GACGCTCTTCCGATCTtcttg | | | | | |
| | tggaaaggacgaaacaccg | | | | | |
| F2S1 | AATGATACGGCGACCACCGAG | 79.1 | 83 | 50.6 | | |
| | ATCTACACTCTTTCCCTACAC | | | | | |
| | GACGCTCTTCCGATCTtcttg | | | | | |
| | tggaaaggacgaaacaccg | | | | | |
| F2S2 | AATGATACGGCGACCACCGAG | 79.1 | 84 | 50 | | |
| | ATCTACACTCTTTCCCTACAC | | | | | |
| | GACGCTCTTCCGATCTTtctt | | | | | |
| | gtggaaaggacgaaacaccg | | | | | |
| F2S3 | AATGATACGGCGACCACCGAG | 79.2 | 85 | 50.6 | | |
| | ATCTACACTCTTTCCCTACAC | | | | | |
| | GACGCTCTTCCGATCTGTAtc | | | | | |
| | ttgtggaaaggacgaaacacc | | | | | |
| | g | | | | | |
| F2S4 | AATGATACGGCGACCACCGAG | 79.2 | 86 | 51.2 | | |
| | ATTACACTCTTTCCCTACACC | | | | | |
| | GACGCTCTTCCGATCTCGTAt | | | | | |
| | cttgtggaaaggacgaaacac | | | | | |
| | cg | | | | | |
| F2S5 | AATGATACGGCGACCACCGAG | 79.2 | 87 | 50.6 | | |
| | ATCTACACTCTTTCCCTACAC | | | | | |
| | GACGCTCTTCCGATCTACGTA | | | | | |
| | tcttgtggaaaggacgaaaca | | | | | |
| | ccg | | | | | |
| F2S6 | AATGATACGGCGACCACCGAG | 79.3 | 88 | 51.1 | | |
| | ATCTACACTCTTTCCCTACAC | | | | | |
| | GACGCTCTTCCGATCTGACGT | | | | | |
| | Atcttgtggaaaggacgaaac | | | | | |
| | accg | | | | | |
| F2S7 | AATGATACGGCGACCACCGAG | 79.1 | 89 | 50.6 | | |
| | ATCTACACTCTTTCCCTACAC | | | | | |
| | GACGCTCTTCCGATCTAGACG | | | | | |
| | TAtcttgtggaaaggacgaaa | | | | | |
| | caccg | | | | | |
| F2S8 | AATGATACGGCGACCACCGAG | 79.3 | 90 | 51.1 | | |
| | ATCTACACTCTTTCCCTACAC | | | | | |
| | GACGCTCTTCCGATCTCAGAC | | | | | |
| | GTAtcttgtggaaaggacgaa | | | | | |
| | acaccg | | | | | |
| F2S9 | AATGATACGGCGACCACCGAG | 79.3 | 91 | 50.5 | | |
| | ATCTACACTCTTTCCCTACAC | | | | | |
| | GACGCTCTTCCGATCTTCAGA | | | | | |
| | CGTAtcttgtggaaaggacga | | | | | |
| | aacaccg | | | | | |
| F2S10 | AATGATACGGCGACCACCGAG | 79.3 | 92 | 51.1 | | |
| | ATCTACACTCTTTCCCTACAC | | | | | |
| | GACGCTCTTCCGATCTGTCAG | | | | | |
| | ACGTAtcttgtggaaaggacg | | | | | |
| | aaacaccg | | | | | |

TABLE S2
List of sgRNAs

| Sequence | |
|---|---|
| Name | Target Sequence |
| COPS7B-1 | TGGCCGTGACATCCGAAAGA |
| COPS7B-2 | TCTTGATGCCAAGCTCACGA |
| COPS7B-3 | TCTTTCAGCAACACGGAGTA |
| COPS7B-4 | GCATCTTACCATCGTGAGCT |
| COPS7B-5 | TTGTTGAACCTGTTTGCCTA |
| COPS7B-6 | GTGGTTCTCTTTGTACTGGT |
| COPS2-1 | GTGGTGGTAAAATGCACTTG |
| COPS2-2 | TAGTAACTCCGAGCCAAATG |
| COPS2-3 | CCAGTTACATCAGTCGTGCC |
| COPS2-4 | GTGGTTTAAGACAAACACAA |
| DDB1-1 | CATTGTCGATATGTGCGTGG |
| DDB1-2 | GGATAGCCATCTGAATTGAG |
| DDB1-3 | CTACCAACCTGCGATCACCA |
| DDB1-4 | TCGTGTCTTGGACTTCAATG |
| COPS8-1 | AGTATACGCTTGAGAGACCA |
| COPS8-2 | AGAAGCTGACCATACACTGG |
| COPS8-3 | CAACCATCAACGCTCACCAG |
| COPS8-4 | GCAGGCAAATTCTGAACTTG |
| Non-Targeting | ATCAGCCCATTTCTGCGCAC |
| Conrol 1 | |
| Non-Targeting | AGGGGCAGGGCTATCTTATG |
| Control 2 | |
| Non-Targeting | GCACATCGTTATATACCAGA |
| Control 3 | |
| Non-Targeting | CAGGGTTGCGCAGAGGACTC |
| Control 4 | |
| OR2H1 | GATGGCCTTTGACCGATACG |
| OR12D2 | GATGGCATTTGACCTCTCTG |
| OR2S2 | GAGAAGGAGATGGTTTCCTG |
| OR5AU1 | GATGAGATAGCACTCACTGG |
| OR5V1 | GAAACCAGCAGCCCAGCATG |
| OR10G2 | GGAGGCTTCTTAGATTTGGG |
| TCEB1 sg2 | GAGCAGCGGCTGTACAAGGT |
| TCEB2 sg3 | GCTTCACCAGTCAAACAGCA |
| CUL2 sg1 | AGATATCTATGCTTTATGTG |
| FBXW2 sg3 | CAGCATGTGAGTAAAGTCTG |
| UBE2R2 sg2 | GGAATCCTACTCAGAATGTG |
| CRBN sg1 | ACCAATGTTCATATAAATGG |
| CRBN sg2 | CTGACTGTGTTCTTAGCTCA |
| CRBN sg3 | TGAAGAGGTAATGTCTGTCC |

TABLE S3
KEY RESOURCES TABLE

| | | |
|---|---|---|
| REAGENT or RESOURCE | SOURCE | IDENTIFIER |

Antibodies

| | | |
|---|---|---|
| Mouse monoclonal anti- c-Myc | Santa Cruz | Cat# sc-40 |
| | Biotechnology | |
| Rabbit polyclonal anti-BRD2 | Bethyl Laboratories | Cat# A302-583A |
| Rabbit polyclonal anti-BRD3 | Bethyl Laboratories | Cat# A302-368A |
| Rabbit polyclonal anti-BRD4 | Bethyl Laboratories | Cat# A301-985A-M |
| Rabbit monoclonal anti-CDK9 (C12F7) | Cell Signaling | Cat# 2316 |
| | Technology | |
| Rabbit monoclonal anti-GAPDH (14C10) | Cell Signaling | Cat# 3683 |
| HRP conjugated | Technology | |
| Horse anti-mouse HRP conjugated | Cell Signaling | Cat# 7076S |
| | Technology | |
| Goat anti-rabbit HRP conjugated | Cell Signaling | Cat# 7074P2 |
| | Technology | |
| Annexin V-FITC | BD Biosciences | Cat# 556420 |
| Biological Samples | | |
| Patient-derived bone marrow samples | Jerome Lipper Multiple | N/A |
| | Myeloma Center - | |
| | Dana Farber Cancer | |
| | Institute | |

Chemicals, Peptides, and Recombinant Proteins

| | | |
|---|---|---|
| Propidium Iodide staining solution | BD Biosciences | Cat# 556463 |
| dBET6 | J Bradner's lab | N/A |
| MZ-1 | Tocris Bioscience | Cat# 61545 |
| ARV-771 | MedChemexpress | Cat# HY-100972 |
| JQ1 | J. Bradner's lab | N/A |
| Bortezomib | Thermo Fisher | Cat# 507419 |
| | Scientific | |
| Thal-SNS-032 | N Gray's lab | N/A |
| Human recombinant interleukin 6 (IL6) | Thermo Fisher | Cat# # |
| | Scientific | 10395HNAE25 |

Critical Commercial Assays

| | | |
|---|---|---|
| Blood & Cell Culture DNA Midi Kit | Qiagen | Cat# 13343 |
| Blood & Cell Culture DNA Maxi Kit | Qiagen | Cat# 13362 |
| QIAquick Gel Extraction Kit | Qiagen | Cat# 28706 |

Deposited Data
Experimental Models: Cell Lines

| | | |
|---|---|---|
| MM1.S | ATCC | Cat# CRL-2974 |
| RPMI-8226 | DSMZ | Cat# ACC-538 |
| OPM-1 | Teru Hideshima, | N/A |
| | Anderson Lab, DFCI | |
| OPM-2 | DSMZ | Cat# ACC-50 |
| JJN3 | DSMZ | Cat# ACC-541 |
| L363 | DSMZ | Cat# ACC-49 |
| AMO-1 | DSMZ | Cat# ACC-538 |
| OCI-My5 | Ontario Cancer | N/A |
| | Institute (OCI); | |
| | Toronto; Canada | |
| HS27A | ATCC | Cat# 50188912FP |
| OPM-2 shRNA CRBN and non-targeting control | K Stewart's lab | N/A |
| KMS11 shRNA CRBN and non-targeting control | K Stewart's lab | N/A |
| MM.1S CRBN⁺ | W Kaelin's lab | N/A |
| MM.1S-Cas9 | B Ebert's lab | N/A |

Experimental Models: Organisms/Strains

| | | |
|---|---|---|
| NOD.Cg-Prkdc^{scid} Il2rg^{tm1Wjl}/SzJ | The Jackson | Cat# 005557 |
| | Laboratory | |

Oligonucleotides

| | |
|---|---|
| List of primers - see Table S1 | IDT |
| List of sgRNAs for single-gene knock-out - see Table S2 | IDT |

Recombinant DNA

| | | |
|---|---|---|
| lentiCas9-Blast | Addgene | Addgene Cat# |
| | | 52962 |
| lentiGuide-Puro | Addgene | Addgene Cat# |
| | | 52963 |
| psPAX2 | Addgene | Addgene |
| | | Plasmid#12250 |
| pMD2.G | Addgene | Addgene Cat |
| | | #12259 |
| GeCKO v2 human library | Addgene | Zhang Lab, Broad |
| | | Institute of MIT and |
| | | Harvard |
| Lenti dCAS9-VP64_Blast | Addgene | Addgene Cat#61425 |
| Brunello human library | Addgene | Addgene Cat#73179 |
| Human CRISPR Activation Pooled sgRNA Library | Addgene | Addgene |
| (Calabrese library) | | Cat#1000000111 |

Software and Algorithms

| | | |
|---|---|---|
| MAGeCK | Li et al., 2014 | https://sourceforge.net/ |
| | | projects/mageck/ |
| PRISM 6 | GraphPad | https://www.graphpad.com |
| FlowJo V9.7.6 | Tree Star | https://www.flowjo.com/ |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of decreasing the viability of a population of cancer cells comprising contacting the cancer cells with a first heterobifunctional proteolysis-targeting chimera (PROTAC) that recruits an E3 ubiquitin ligase to an oncogenic protein and sequentially contacting the cancer cells with a second heterobifunctional PROTAC that recruits a different E3 ubiquitin ligase to the oncogenic protein, thereby decreasing the viability of the cancer cells.

2. A method of delaying or preventing resistance of a population of cancer cells to an anti-oncogenic protein therapy comprising contacting the cancer cells with a first heterobifunctional proteolysis-targeting chimera (PROTAC) that recruits an E3 ubiquitin ligase to an oncogenic protein and sequentially contacting the cancer cells with a second heterobifunctional PROTAC that recruits a different E3 ubiquitin ligase to the oncogenic protein, thereby decreasing the viability of the cancer cells.

3. A method of decreasing the viability of a population of cancer cells previously contacted with a first heterobifunctional proteolysis-targeting chimera (PROTAC) that recruits an E3 ubiquitin ligase to an oncogenic protein comprising contacting the cancer cells with a second heterobifunctional PROTAC that recruits a different E3 ubiquitin ligase to the oncogenic protein, thereby decreasing the viability of the cancer cells.

4. A method of delaying or preventing resistance of a population of cancer cells to an anti-oncogenic protein therapy comprising contacting the cancer cells with a heterobifunctional PROTAC that recruits an E3 ubiqutin ligase to the oncogenic protein, wherein the cancer cells were previously contacted with a heterobifunctional PROTAC that recruits a different E3 ubiqutin ligase to the oncogenic protein.

5. The method of any one of claims 1-4, wherein the oncogenic protein is selected from the group consisting of CDK9, BRD2, BRD3, and BRD4.

6. The method of any one of claims 1-5, wherein the E3 ubiquitin ligase is selected from the group consisting of CRBN, VHL, MDM2, APC/C, KCMF1, and RNF4.

7. The method of any one of claims 1-6, wherein the heterobifunctional PROTAC is selected from the group consisting of dBET6, Thal-SNS-032, ARV-771, and MZ-1.

8. The method of any one of claims 1-7, wherein the heterobifunctional PROTAC agents do not contact the cancer cells at the same time.

9. The method of any one of claims 1-8, wherein at least one additional cancer treatment contacts the cancer cells at the same time as at least one of the heterobifunctional PROTAC agents.

10. The method of claim 9, wherein the additional cancer treatment is selected from the group consisting of immunotherapy, targeted therapy, chemotherapy, radiation therapy, hormonal therapy, an anti-cancer vaccine, an anti-cancer virus, a checkpoint inhibitor, radiosensitizer, and combinations thereof.

11. The method of any one of claims 1-10, wherein the cancer cells are anti-cancer therapy naïve cancer cells.

12. The method of any one of claims 1-11, wherein the step of contacting occurs in vivo, ex vivo, or in vitro.

13. The method of any one of claims 1-12, wherein the cancer cells are multiple myeloma cells.

14. The method of any one of claims 1-13, wherein the cancer cells are in a subject and the subject is an animal model of the cancer.

15. The method of claim 14, wherein the animal model is a rodent or primate model.

16. The method of any one of claims 1-13, wherein the cancer cells are in a subject and the subject is a mammal.

17. The method of claim 16, wherein the mammal is a rodent, a primate, or a human.

18. The method of any one of claims 1-17, wherein between contacting the cancer cells with the first heterbifunctional PROTAC agent and the second heterobifunctional PROTAC agent, the cancer cells and/or subject have undergone cancer treatment, completed treatment, and/or are in remission for the cancer.

19. The method of claim 18, wherein the subject has multiple myeloma.
